(19) United States
(12) Patent Application Publication
(10) Pub. No.: US 2024/0141440 A1
(43) Pub. Date: May 2, 2024

(54) METHODS FOR DETECTING ONCOGENIC KRAS MUTATIONS

(71) Applicant: Duke University, Durham, NC (US)

(72) Inventors: Christopher Counter, Durham, NC (US); Siqi Li, Durham, NC (US)

(21) Appl. No.: 18/499,880

(22) Filed: Nov. 1, 2023

Related U.S. Application Data
(60) Provisional application No. 63/421,491, filed on Nov. 1, 2022.

(51) Int. Cl. C12Q 1/6886; C12Q 1/6874

(52) U.S. Cl. CPC C12Q 1/6886; C12Q 1/6874; C12Q 1/6806; C12Q 2600/156; C12Q 2600/16

(57) ABSTRACT

The present invention provides methods and kits for the detection of rare genetic mutations in the human KRAS gene. The methods provided herein use specific restriction enzymes that target regions in the 3′ end of a region of interest within the human KRAS gene paired with primers designed for the specific amplification of the digested regions of interest to identify samples comprising these rare mutations.

### CROSS-REFERENCE TO RELATED APPLICATIONS

This application claims priority to U.S. Provisional Application No. 63/421,491 filed on Nov. 1, 2022, the content of which is incorporated by reference in its entirety.

### STATEMENT REGARDING FEDERALLY SPONSORED RESEARCH

This invention was made with government support under grant numbers R01CA94184 and P01CA203657 awarded by the National Institute of Cancer and under grant number R35GM127062 awarded by the National Institute of General Medicine. The government has certain rights in this invention.

### SEQUENCE LISTING

A Sequence Listing accompanies this application and is submitted as an ASCII text file of the sequence listing named “155554_00719_Sequence_Listing.xml” which is 197,938 bytes in size and was created on Oct. 31, 2023. The sequence listing is electronically submitted via Patent Center with the application and is incorporated herein by reference in its entirety.

### BACKGROUND

The three human Ras genes, i.e., KRAS, HRAS, and NRAS, are the most common oncogenes detected in human cancer. KRAS mutations are found at high rates in leukemias, colorectal cancer, pancreatic cancer and lung cancer. Conventional next generation sequencing (NGS) methods are used to detect KRAS mutations, but they have a detection limit of about one mutant copy per 1000 gene copies, whereas a mutation detected by the method may be present in the sample at a frequency much lower than one mutant copy per 1000 gene copies, and even lower than one mutant copy per 2×10⁶ gene copies. Therefore, improved sequencing methods are needed to detect these rare mutations.

### SUMMARY

The present disclosure provides methods and kits for detecting a mutation in a Ras gene in a human sample.

In an aspect, provided herein is a method of detecting a mutation in a KRAS gene in a sample from a human subject, the method comprising: (a) digesting genomic DNA in the sample with at least one enzyme that cleaves at the 3′ end of a region of interest (ROI); (b) adding a forward adaptor and a barcode to the 3′ end of the ROI by mixing the digested genomic DNA with an adaptor-barcode primer and performing a single round of extension, wherein the adaptor-barcode primer comprises from 5′ to 3′: a forward adaptor sequence, a barcode sequence, and a first ROI-specific sequence that is complementary to the 3′ end of the digested ROI, wherein adding the forward adaptor and the barcode to the ROI produces a barcoded DNA; (c) performing linear amplification of the barcoded DNA produced in (b) using a forward adaptor primer that anneals to the forward adaptor sequence; (d) performing exponential amplification of the linearly amplified barcoded DNA produced in (c) using: an exon-specific reverse primer comprising from 5′ to 3′: a reverse adaptor sequence, and a second ROI-specific sequence that is complementary to the 5′ end of the digested ROI; the forward adaptor primer; and a reverse adaptor primer that anneals to the reverse adaptor sequence; and (e) sequencing the exponentially amplified barcoded DNA produced in (d); wherein a different nucleotide in the sequenced DNA produced in (e) compared to a wild type KRAS sequence indicates that a mutation is detected.

In embodiments, the ROI is on the transcribed strand of KRAS exon 1, the non-transcribed strand of KRAS exon 1, or the non-transcribed strand of KRAS exon 2.

In embodiments, the ROI is on the transcribed strand of KRAS exon 1; and: the enzyme is StuI, the adaptor-barcode primer comprises the first ROI-specific sequence of SEQ ID NO: 6, and the exon-specific reverse primer comprises the second ROI-specific sequence of SEQ ID NO: 7 or SEQ ID NO: 8; the enzyme is Hinfl, the adaptor-barcode primer comprises the first ROI-specific sequence of SEQ ID NO: 25, and the exon-specific reverse primer comprises the second ROI-specific sequence of SEQ ID NO: 17; the enzyme is MluCI, the adaptor-barcode primer comprises the first ROI-specific sequence of SEQ ID NO: 26, and the exon-specific reverse primer comprises the second ROI-specific sequence of SEQ ID NO: 17; the enzyme is Hpy188I, the adaptor-barcode primer comprises the first ROI-specific sequence of SEQ ID NO: 27, and the exon-specific reverse primer comprises the second ROI-specific sequence of SEQ ID NO: 17; the enzyme is AlwI, the adaptor-barcode primer comprises the first ROI-specific sequence of SEQ ID NO: 28, and the exon-specific reverse primer comprises the second ROI-specific sequence of SEQ ID NO: 17; the enzyme is DpnII, the adaptor-barcode primer comprises the first ROI-specific sequence of SEQ ID NO: 29, and the exon-specific reverse primer comprises the second ROI-specific sequence of SEQ ID NO: 17; the enzyme is MnlI, the adaptor-barcode primer comprises the first ROI-specific sequence of SEQ ID NO: 30, and the exon-specific reverse primer comprises the second ROI-specific sequence of SEQ ID NO: 17; the enzyme is NsiI, the adaptor-barcode primer comprises the first ROI-specific sequence of SEQ ID NO: 31, and the exon-specific reverse primer comprises the second ROI-specific sequence of SEQ ID NO: 17; the enzyme is HpyCH4V, the adaptor-barcode primer comprises the first ROI-specific sequence of SEQ ID NO: 32, and the exon-specific reverse primer comprises the second ROI-specific sequence of SEQ ID NO: 17; or the enzyme is BsrI, the adaptor-barcode primer comprises the first ROI-specific sequence of SEQ ID NO: 33, and the exon-specific reverse primer comprises the second ROI-specific sequence of SEQ ID NO: 17.

In embodiments, the ROI is on the non-transcribed strand of KRAS exon 1; and: the enzyme is HinfI, the adaptor-barcode primer comprises the first ROI-specific sequence of SEQ ID NO: 9, and the exon-specific reverse primer comprises the second ROI-specific sequence of SEQ ID NO: 10; the enzyme is PsiI, the adaptor-barcode primer comprises the first ROI-specific sequence of SEQ ID NO: 18, and the exon-specific reverse primer comprises the second ROI-specific sequence of SEQ ID NO: 8; the enzyme is Tsp45I, the adaptor-barcode primer comprises the first ROI-specific sequence of SEQ ID NO: 19, and the exon-specific reverse primer comprises the second ROI-specific sequence of SEQ ID NO: 8; the enzyme is selected from AflIII, PciI and FatI, the adaptor-barcode primer comprises the first ROI-specific sequence of SEQ ID NO: 20, and the exon-specific reverse primer comprises the second ROI-specific sequence of SEQ ID NO: 8; the enzyme is selected from NspI and NlaIII, the adaptor-barcode primer comprises the first ROI-specific sequence of SEQ ID NO: 21, and the exon-specific reverse primer comprises the second ROI-specific sequence of SEQ ID NO: 8; the enzyme is CviAII, and the adaptor-barcode primer comprises the first ROI-specific sequence of SEQ ID NO: 22, and the exon-specific reverse primer comprises the second ROI-specific sequence of SEQ ID NO: 8; the enzyme is CviQ, the adaptor-barcode primer comprises the first ROI-specific sequence of SEQ ID NO: 23, and the exon-specific reverse primer comprises the second ROI-specific sequence of SEQ ID NO: 8; or the enzyme is HphI, the adaptor-barcode primer comprises the first ROI-specific sequence of SEQ ID NO: 24, and the exon-specific reverse primer comprises the second ROI-specific sequence of SEQ ID NO: 8.

In embodiments, the ROI is on the non-transcribed strand of KRAS exon 2; and the enzyme is XmnI, the adaptor-barcode primer comprises the first ROI-specific sequence of SEQ ID NO: 11, and the exon-specific reverse primer comprises the second ROI-specific sequence of SEQ ID NO: 12.

In embodiments, the adaptor-barcode primer comprises the forward adaptor sequence of SEQ ID NO: 1; and wherein the forward adaptor primer comprises SEQ ID NO: 2. In embodiments, the exon-specific reverse primer comprises the reverse adaptor sequence of SEQ ID NO: 3; and wherein the reverse adaptor primer comprises SEQ ID NO: 4.

In embodiments, the adaptor-barcode primer further comprises a first index sequence between the forward adaptor sequence and the barcode sequence; wherein the exon-specific reverse primer further comprises a second index sequence between the reverse adaptor sequence and the second ROI-specific sequence; and wherein the first index sequence and the second index sequence comprise between one and seven nucleotides. In embodiments, the first index sequence and the second index sequence are selected from A, GA, CGA, TCGA, ATCGA, GATCGA, and CGATCGA.

In embodiments, the adaptor-barcode primer comprises a sequence selected from SEQ ID NOs: 37 and 41-111. In embodiments, the exon-specific reverse primer comprises a sequence selected from SEQ ID NOs: 38 and 112-118.

In embodiments, the adaptor-barcode primer comprises a sequence selected from SEQ ID NOs: 34 and 119-181. In embodiments, the exon-specific reverse primer comprises a sequence selected from SEQ ID NOs: 36 and 182-188.

In embodiments, performing exponential amplification in step (d) comprises performing at least 20 PCR cycles.

In embodiments, the sample is a biopsy. In embodiments, the subject has or is suspected of having cancer.

In another aspect, provided herein is a kit for detecting a mutation in a KRAS gene in a human subject, the kit comprising at least one set of primers comprising: a forward adaptor primer comprising SEQ ID NO: 2; a reverse adaptor primer comprising SEQ ID NO: 4; an adaptor barcode primer selected from SEQ ID NOs: 34, 37, 39, 41-111, and 119-181; and an exon-specific reverse primer selected from SEQ ID NOs: 35, 38, 40, 112-118, and 182-188; wherein each of the at least one set of primers comprises an adaptor barcode primer and an exon-specific barcode primer that comprise a sequence that targets the same region of interest in KRAS.

In embodiments, the kit further comprises at least one enzyme selected from StuI, HinfI, AlwI, BsrI, DpnII, Hpy188I, HpyCH4V, MluCI, MnlI, NsiI, StuI, AflIII, PciI, FatI, NlaIII, CviAII, CviQI, HphI, NspI, PsiI, XmnI, and Tsp45I; wherein the at least one enzyme cleaves KRAS at the 3′ end of the region of interest that the adaptor barcode primer and the exon-specific barcode primer target.

In embodiments, the enzyme is StuI, the adaptor-barcode primer comprises the first ROI-specific sequence of SEQ ID NO: 6, and the exon-specific reverse primer comprises the second ROI-specific sequence of SEQ ID NO: 7 or SEQ ID NO: 8; the enzyme is Hinfl, the adaptor-barcode primer comprises the first ROI-specific sequence of SEQ ID NO: 25, and the exon-specific reverse primer comprises the second ROI-specific sequence of SEQ ID NO: 17; the enzyme is MluCI, the adaptor-barcode primer comprises the first ROI-specific sequence of SEQ ID NO: 26, and the exon-specific reverse primer comprises the second ROI-specific sequence of SEQ ID NO: 17; the enzyme is Hpy188I, the adaptor-barcode primer comprises the first ROI-specific sequence of SEQ ID NO: 27, and the exon-specific reverse primer comprises the second ROI-specific sequence of SEQ ID NO: 17; the enzyme is AlwI, the adaptor-barcode primer comprises the first ROI-specific sequence of SEQ ID NO: 28, and the exon-specific reverse primer comprises the second ROI-specific sequence of SEQ ID NO: 17; the enzyme is DpnII, the adaptor-barcode primer comprises the first ROI-specific sequence of SEQ ID NO: 29, and the exon-specific reverse primer comprises the second ROI-specific sequence of SEQ ID NO: 17; the enzyme is MnlI, the adaptor-barcode primer comprises the first ROI-specific sequence of SEQ ID NO: 30, and the exon-specific reverse primer comprises the second ROI-specific sequence of SEQ ID NO: 17; the enzyme is NsiI, the adaptor-barcode primer comprises the first ROI-specific sequence of SEQ ID NO: 31, and the exon-specific reverse primer comprises the second ROI-specific sequence of SEQ ID NO: 17; the enzyme is HpyCH4V, the adaptor-barcode primer comprises the first ROI-specific sequence of SEQ ID NO: 32, and the exon-specific reverse primer comprises the second ROI-specific sequence of SEQ ID NO: 17; the enzyme is BsrI, the adaptor-barcode primer comprises the first ROI-specific sequence of SEQ ID NO: 33, and the exon-specific reverse primer comprises the second ROI-specific sequence of SEQ ID NO: 17; the enzyme is HinfI, the adaptor-barcode primer comprises the first ROI-specific sequence of SEQ ID NO: 9, and the exon-specific reverse primer comprises the second ROI-specific sequence of SEQ ID NO: 10; the enzyme is PsiI, the adaptor-barcode primer comprises the first ROI-specific sequence of SEQ ID NO: 18, and the exon-specific reverse primer comprises the second ROI-specific sequence of SEQ ID NO: 8; the enzyme is Tsp45I, the adaptor-barcode primer comprises the first ROI-specific sequence of SEQ ID NO: 19, and the exon-specific reverse primer comprises the second ROI-specific sequence of SEQ ID NO: 8; the enzyme is selected from AflIII, PciI and FatI, the adaptor-barcode primer comprises the first ROI-specific sequence of SEQ ID NO: 20, and the exon-specific reverse primer comprises the second ROI-specific sequence of SEQ ID NO: 8; the enzyme is selected from NspI and NlaIII, the adaptor-barcode primer comprises the first ROI-specific sequence of SEQ ID NO: 21, and the exon-specific reverse primer comprises the second ROI-specific sequence of SEQ ID NO: 8; the enzyme is CviAII, and the adaptor-barcode primer comprises the first ROI-specific sequence of SEQ ID NO: 22, and the exon-specific reverse primer comprises the second ROI-specific sequence of SEQ ID NO: 8; the enzyme is CviQ, the adaptor-barcode primer comprises the first ROI-specific sequence of SEQ ID NO: 23, and the exon-specific reverse primer comprises the second ROI-specific sequence of SEQ ID NO: 8; the enzyme is HphI, the adaptor-barcode primer comprises the first ROI-specific sequence of SEQ ID NO: 24, and the exon-specific reverse primer comprises the second ROI-specific sequence of SEQ ID NO: 8; or the enzyme is XmnI, the adaptor-barcode primer comprises the first ROI-specific sequence of SEQ ID NO: 11, and the exon-specific reverse primer comprises the second ROI-specific sequence of SEQ ID NO: 12.

### BRIEF DESCRIPTION OF THE DRAWINGS

FIGS. 1A-1F demonstrates that MDS detects ultra-rare mutations induced by the carcinogen urethane. A) Frequency of single (detected) versus co-occurring (present) mutations identified by MDS using a dilution series of Kras cDNAs with 2-3 different mutations engineered in exon 1 mixed with genomic DNA from mouse lung tissue. B-D) Heatmap of the mutation frequency (MF) determined by MDS for the non-transcribed strand of exon 2 of Kras from the lungs of mice at the indicated time points after exposure to urethane (UR) or PBS (n=7 mice for the UR and PBS cohorts at week 1, 5 mice for the PBS cohort at week 4, and 4 mice for all other cohorts from one experiment), plotted as B, C) the log-transformed fold-change normalized to PBS-treated mice (FC over PBS) or D) log transformed versus each B) nucleotide (annotated by amino acid at the top, Q₆₁L and R mutations are highlighted in red, scaled by detected frequency), C) type of substitution or, D) A>T transversions (nucleotide number as well as the 5′ and 3′ base of the substituted A are shown at the top). E, F) Mean±SEM mutation frequency of E) each possible CAN to CTN transversion at the indicated time points after mice were exposed to urethane (UR) or PBS (n=7 mice for the UR and PBS cohorts at week 1, 5 mice for the PBS cohort at week 4, and 4 mice for all other cohorts from one experiment) or F) all possible missense mutations for Q₆₁ codon in mice 1 week after urethane exposure (n=7 mice from one experiment). p<values calculated by E) Dunn's multiple comparison test following Kruskal-Wallis test or F) Holm-Sidak multiple comparisons test following one-way ANOVA. ****p<0.0001 and ***p<0.001.

FIGS. 2A-2D demonstrates that MDS detects the position and substitution tropism of urethane. A, B) Heatmap of the mutation frequency (MF) determined by MDS for the transcribed strand of exon 1 of Kras from the lungs of mice at the indicated time points after exposure to urethane (UR) or PBS (n=2 mice for the UR cohort at week 2, 3 mice for the UR and PBS cohorts at week 3, and 4 mice for all other cohorts from one experiment), plotted as the A) log-transformed fold-change normalized to PBS-treated mice (FC over PBS) or B) log transformed versus each A) type of substitution or B) A>T transversions (nucleotide number as well as the 5′ and 3′ base of the substituted A are shown at the top). C, D) Mean±SEM mutation frequency for all possible oncogenic mutations at G₁₂ and G₁₃ (n=4 mice from one experiment) compared to the previous determined mutation frequency at codon Q₆₁ (FIG. 1, n=7 mice at week 1 and 4 mice at week 4 from one experiment) C) 1 week or D) 4 weeks after urethane exposure. p values calculated by Holm-Sidak multiple comparisons test following one-way ANOVA. ****p<0.0001, ***p<0.001, **p<0.01, and *p<0.05.

FIGS. 3A-3D demonstrates that MDS detects the isoform tropism of urethane. A, B) Heatmap of the mutation frequency (MF) determined by MDS for the non-transcribed strand of exon 2 of Hras from the lungs of mice at the indicated time points after exposure to urethane (UR) or PBS (n=4 mice at each time point from one experiment), plotted as the A) log-transformed fold-change normalized to PBS-treated mice (FC over PBS) or B) log transformed versus each A) type of substitution or B) A>T transversions (nucleotide number as well as the 5′ and 3′ base of the substituted A are shown at the top). C, D) Mean±SEM mutation frequency of each possible CAN to CTN transversion in exon 2 of Hras (n=4 mice from one experiment) compared to the previous determined mutation frequency in exon 2 of Kras (FIG. 1, n=7 mice at week 1 and 4 mice at week 4 from one experiment) at C) 1 week or D) 4 weeks after exposure to urethane (UR) or PBS. p values calculated by C) Holm-Sidak multiple comparisons test following one-way ANOVA, or D) Dunn's multiple comparison test following Kruskal-Wallis test. ****p<0.0001 and ns: not significant.

FIGS. 4A-4B demonstrates that MDS detects the organ tropism of urethane. A, B) Mean±SEM mutation frequency each possible CAN to CTN transversion determined by MDS for the non-transcribed strand of exon 2 of Kras in the pancreas or liver of mice (n=3 mice for pancreas samples from the UR cohort at weeks 1 and 4, 5 mice for liver samples from the UR and PBS cohorts at week 1, and 4 mice for all other cohorts from one experiment) compared to the previous determined mutation frequency in the lung (FIG. 1, n=7 mice at week 1 and 4 mice at week 4 from one experiment) either A) 1 week or B) 4 weeks after exposure to urethane (UR) or PBS. p values calculated by A) Holm-Sidak multiple comparisons test following one-way ANOVA or B) Dunn's multiple comparison test following Kruskal-Wallis test. ****p<0.0001, **p<0.01, and ns: not significant.

FIGS. 5A-5E demonstrates that MDS detects urethane strand bias. A, B, D) Mean±SEM mutation frequency of the indicated CAto CTN transversions and reverse-complementary substitutions averaged by nucleotide positions determined by MDS sequencing of Kras exon 2 A) non-transcribed strand in lungs (FIG. 1, n=7 mice from one experiment) or D) non-transcribed strand in livers of mice 1 week (n=5 mice from one experiment) or B) transcribed strand in lungs of mice 3 weeks (n=4 mice from one experiment) after exposure to urethane (UR) or PBS. C) Mean±SEM relative expression of Kras mRNA in the lung, liver, and pancreas (normalized to lung) of mice determined by RT-qPCR (n=4 mice from one experiment). E) Mean±SEM frequency of CAto CTN transversions of the non-transcribed versus transcribed strand in urethane-induced tumors from whole exome sequencing data³ in genes binned by their mRNA levels from the mouse lung³⁶ (n=66 tumors). p values calculated by Holm-Sidak multiple comparisons test following A, B, D, E) two-way ANOVA or C) one-way ANOVA. ****p<0.0001, *p<0.05 and ns: not significant.

FIGS. 6A-6D demonstrates that MDS was optimized to detect ultra-rare mutations in the mammalian genome. A) Diagram of MDS assay optimized for the transcribed strand of exon 1 of Kras based on Jee, J. et al. (Nature 534, 693-6 (2016). B, C) Frequency of single (detected) versus co-occurring (present) mutations identified by MDS using a dilution series of Kras cDNAs with 2-3 different mutations engineered in B) exon 1 or C) exon 2 mixed with genomic DNA from MEFs. D) Mean±SEM frequency of Kras^{Q61L} mutations detected in the lungs of mice 4 weeks after exposure to urethane (UR) by MDS targeting the non-transcribed strand of exon 2 of Kras versus droplet digital PCR (ddPCR) (n=4 mice for MDS and 3 mice for ddPCR from one experiment).

FIGS. 7A-7C demonstrates that MDS detects urethane mutation specificity. A) Heatmap of the mutation frequency (MF) determined by MDS for the transcribed strand of exon 2 of Kras from the lungs of mice 3 weeks after exposure to urethane (UR) or PBS (n=4 mice from one experiment), plotted as log transformed versus each A>T transversions (nucleotide number as well as the 5′ and 3′ base of the substituted A are shown at the top). B) Log10 p value of substitutions (shown as nucleotide position_substitution_trinucleotide) identified by MDS targeting the non-transcribed strand of Kras exon 2 that are significantly increased in mice exposed to urethane (UR) compared to PBS (FIG. 1, n=19 mice for UR and 20 mice for PBS from one experiment). p value calculated by two-tailed Mann-Whitney U test comparing all samples from urethane-exposed mice with all samples from PBS-exposed mice from 1 to 4 weeks. Dotted line: p=0.05. C) Mean±SEM mutation frequency of each possible CAN to CGN transitions at the indicated time points after mice were exposed to urethane (UR) or PBS (FIG. 1, n=7 mice at 1 week and 4 mice at 4 week from one experiment). p values calculated by Dunn's multiple comparison test following Kruskal-Wallis test. ****p.

FIGS. 8A-8B demonstrates that MDS detects the substitution tropism of urethane. A) Heatmap of the mutation frequency (1VIF) determined by MDS for the non-transcribed strand of exon 1 of Kras from the lungs of mice at the indicated time points after exposure to urethane (UR) or PBS (n=4 mice for UR at 1 and 4 week, 5 mice for PBS at 1 week, and 3 mice for PBS at 4 week from one experiment), plotted as log transformed versus each A>T transversions (nucleotide number as well as the 5′ and 3′ base of the substituted A are shown at the top). B) Log10 p value of substitutions (shown as nucleotide position_substitution_trinucleotide) identified by MDS targeting the transcribed strand of Kras exon 1 that are significantly increased in mice exposed to urethane (UR) compared to PBS (FIG. 2, n=13 mice for UR and 11 mice for PBS from one experiment). p value calculated by two-tailed Mann-Whitney U test comparing all samples from urethane-exposed mice with all samples from PBS-exposed mice from 1 to 4 weeks. Dotted line: p=0.05.

FIGS. 9A-9B shows the distribution of urethane and its metabolite across tissues. Mean±SEM concentration of A) urethane or B) vinyl carbamate at the indicated time points after mice were injected with urethane in indicated tissues determined by LC/MS/MS (n=4 mice from one experiment). p values calculated by Holm-Sidak multiple comparisons test following two-way ANOVA. ****p<0.05.

FIGS. 10A-10C demonstrates that MDS detects urethane strand bias. A, B) Mean±SEM mutation frequency of the indicated CAto CTNtransversions and reverse-complementary substitutions averaged by nucleotide positions determined by MDS sequencing for Kras exon 1 A) non-transcribed strand (n=4 mice for UR and 5 mice for PBS from one experiment) or B) transcribed strand (FIG. 2, n=4 mice from one experiment) from the lungs of mice 1 week after exposure to urethane (UR) or PBS. C) Mean±SEM frequency of CAto CTN transversions of the non-transcribed versus transcribed strand in urethane-induced tumors from whole exome sequencing data3 in genes binned by their mRNA levels from the mouse lung reported in the mouse ENCODE project³⁷ (n=66 tumors). p values calculated by Holm-Sidak multiple comparisons test following two-way ANOVA. ****p.

FIG. 11 is a schematic depiction of KRAS MDS. Unique barcodes and an adaptor are introduced into exon 1 of KRAS through restriction digestion (1) and first-strand extension (2), followed by linear amplification to copy the original DNA 12 times (3) and 20 cycles of exponential amplification (4) to expand the library for sequencing (5). Sequencing reads are grouped by barcode and bona fide mutations (filed in circles) separated from false ones (empty circles) by virtue of being present in all reads sharing the same barcode.

FIGS. 12A-12B depicts the detection of ultra-rare mutations in human KRAS. The mutation frequency (detected frequency) of a single mutation engineered in a library of human KRAS mutant DNAs spiked at increasing levels into gDNA isolated from human 293T cells versus the frequency detected with a co-occurring mutation (mutant present) in the A) coding and B) non-coding strand assessed by K-MDS (O). Base changes with typically higher background are denoted in red (O).

FIG. 13 is a barplot showing the frequency of KRAS mutations (as a fraction of reference sequence) detected by a mammalian version of MDS specific for KRAS in two healthy donor blood samples (10 ml) spiked with cancer cell lines (with the indicated KRAS mutations and dilutions) as follows: ASPC-1 (KRAS^{G12D}, 1,000 cells/ml), CFPAC-1 (KRAS^{G12V}, 100 cells/ml), MiaPaCa2 (KRAS^{G12C}, 10 cells/ml), HCT166 (KRAS^{G13D}, 1 cell/ml).

### DETAILED DESCRIPTION

The present invention provides methods and kits for the detection of rare genetic mutations in the human KRAS gene. These methods were adapted from the previously developed, error-corrected, high-throughput sequencing method referred to as maximum depth sequencing (MDS). MDS overcomes the limitations of conventional next generation sequencing (NGS) to allow for the identification of ultra-rare (1×10⁻⁶, 1 mutant per 10⁶ templates) antibiotic-resistance mutations arising in bacteria populations¹³. The inventors adapted the MDS method for use with the much larger mammalian genome.

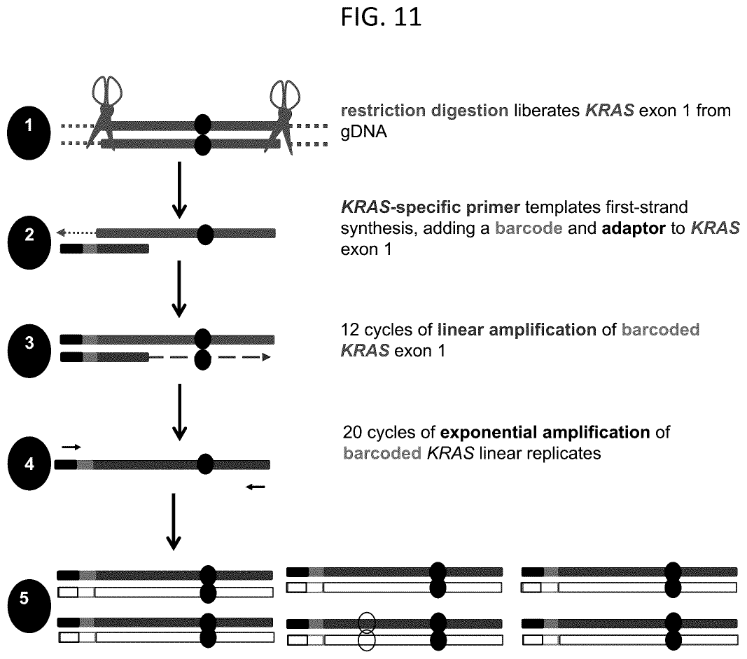

To perform MDS, genomic DNA is cleaved (e.g., using a restriction enzyme) at the 3′ end of a region of interest (ROI) (Step a). Then, a single PCR cycle is performed to anneal “adaptor-barcode primers,” which comprise a forward adaptor sequence and a barcode sequence, to the 3′ end of the ROI (Step b). In this step, the exposed 3′ end of the genomic DNA molecule serves as a “primer” that allows the adaptor sequence and barcode sequence to be synthesized onto the end of the ROI. Next, unused adaptor-barcode primers are removed, and the ROI is subjected to linear amplification using a “forward adaptor primer” that anneals to the newly added forward adaptor sequence (Step c). Next, the DNA is subjected to exponential amplification using an “exon-specific reverse primer” that comprises a reverse adaptor sequence and a “reverse adaptor primer” that anneals to the reverse adaptor sequence (Step d). Finally, the DNA is sequenced, and the results are used to identify mutations in the ROI. See FIG. 11 for a schematic depiction of the optimized MDS assay. The original MDS protocol is described in U.S. Pat. No. 10,513,732 and in Jee, J. et al. (Nature 534, 693-6 (2016)), which are each incorporated by reference in their entirety.

The inventors modified the original MDS protocol developed for bacteria to function in mammals in several ways. Specific restriction enzymes that target a specific region in the 3′ end of a region of interest (ROI) within the human KRAS gene were selected, and primers were designed for the specific amplification of the digested ROIs. Several additional parameters, such as the PCR annealing temperatures and the number of PCR cycles were also optimized for use with these particular ROIs and primers.

### Methods

Provided herein is a method of detecting a mutation in the human KRAS gene in a sample from a subject. The method comprises: (a) digesting genomic DNA in the sample with at least one enzyme that cleaves at the 3′ end of a region of interest (ROI); (b) adding a forward adaptor and a barcode to the 3′ end of the ROI by mixing the digested genomic DNA with an adaptor-barcode primer and performing a single round of extension, wherein the adaptor-barcode primer comprises from 5′ to 3′: a forward adaptor sequence, a barcode sequence, and a first ROI-specific sequence that is complementary to the 3′ end of the digested ROI, wherein adding the forward adaptor and the barcode to the ROI produces a barcoded DNA; (c) performing linear amplification of the barcoded DNA produced in (b) using a forward adaptor primer that anneals to the forward adaptor sequence; (d) performing exponential amplification of the linearly amplified barcoded DNA produced in (c) using (i) an exon-specific reverse primer comprising from 5′ to 3′: a reverse adaptor sequence and a second ROI-specific sequence that is complementary to the 5′ end of the digested ROI, and (ii) a reverse adaptor primer that anneals to the reverse adaptor sequence; and (e) sequencing the exponentially amplified barcoded DNA produced in (d); wherein a different nucleotide in the sequenced DNA produced in (e) compared to a wild type KRAS sequence indicates that a mutation is detected.

The methods of the present invention are designed to detect a mutation, typically a point mutation, within the human KRAS gene. Kras is a member of the Ras family of proteins, which are GTPases that function as molecular switches that control intracellular signaling pathways. Overactive Ras signaling can lead to cancer. The three human Ras genes, i.e., KRAS, HRAS, and NRAS, are the most common oncogenes detected in human cancer. Therefore, the mutation sought may be an oncogenic driver mutation. The methods are designed to detect rare mutations. Conventional next generation sequencing (NGS) methods have a detection limit of about one mutant copy per 1000 gene copies. In contrast, a mutation detected by the method may be present in the sample at a frequency lower than one mutant copy per 1000 gene copies, or even at a frequency lower than one mutant copy per 2×10⁶ gene copies.

The present methods rely on the analysis of short reads within a defined region of interest (ROI). Thus, a specific portion of the KRAS gene must be selected for analysis. Preferably, the ROI is greater than 50 bp and less than 150 bp in size. In the Examples, the inventors disclose methods that can be used to detect mutations in the transcribed strand of KRAS exon 1, the non-transcribed strand of KRAS exon 1, or the non-transcribed strand of KRAS exon 2. Thus, the ROI may be on the transcribed strand of KRAS exon 1, the non-transcribed strand of KRAS exon 1, or the non-transcribed strand of KRAS exon 2.

In Step (a) of the present methods, at least one enzyme is used to digest genomic DNA in the sample. Any enzyme that is capable of cleaving or nicking a genomic DNA at the 3′ end of a ROI may be used in this step. The enzyme may be an endonuclease. Exemplary enzymes include, but are not limited to, StuI, HinfI, AlwI, BsrI, DpnII, Hpy188I, HpyCH4V, MluCI, MnlI, NsiI, StuI, AflIII, PciI, FatI, NlaIII, CviAII, CviQI, HphI, NspI, PsiI, XmnI, and Tsp45I. The enzyme may be a restriction enzyme. In embodiments, enzymatic digestion is performed by CRISPR/Cas9. Notably, since the barcode/adaptor sequences are added to the ROI using DNA polymerization, the method is not limited to only the use of restriction enzymes with overhangs of a specific length, as are required by other approaches.

In Step (b), a single round of extension with an adaptor-barcode primer is used to add a forward adaptor and a barcode to the 3′ end of the ROI. The adaptor-barcode primer is an oligonucleotide that comprises from 5′ to 3′: a forward adaptor sequence, a barcode sequence, and a first ROI-specific sequence. The adaptor-barcode primer may have varying lengths and compositions as required by the protocol. In some cases, more than one adaptor amplifier primer may be used.

Adaptor sequences are designed to interact with a specific sequencing platform (e.g., the surface of a flow-cell (Illumina) or beads (Ion Torrent)) to facilitate a sequencing reaction. Thus, the optimal length of the forward adaptor sequence and the reverse adaptor sequence will vary depending on the sequencing platform used. One of ordinary skill will understand that adaptor sequences may be as short as 20 nucleotides or substantially longer. For example, an adaptor sequence of 58 nucleotides may be used with an Illumina machine.

A barcode sequence is a short, pre-defined sequence that is used to track the origin of specific DNA molecules (i.e., which sample they came from) through the sequencing process. A barcode sequence may be about 6-40 nucleotides in length. In exemplary embodiments, the barcode sequence is 14 nucleotides in length (as in the generic barcode sequence of NNNNNNNNNNNNNN) Multiple barcodes may be used. For example, a second barcoded primer may be used to add a second barcode sequence after linear amplification is performed in Step (b).

The first ROI-specific sequence is a sequence that is complementary to the 3′ end of the digested ROI, allowing the adaptor-barcode primer to anneal to the 3′ end of the digested ROI. The first ROI-specific sequence may be about 8-30 nucleotides in length.

In Step (c) of the present methods, the barcoded DNA is subjected to linear amplification using a single primer to replicate the copies of the same barcoded DNA template. In linear amplification, the same DNA molecule is copied multiple times, which reduces the probability of recovering a defective copy. Linear amplification is accomplished using PCR with a forward adaptor primer that anneals to the forward adaptor sequence. For example, in some embodiments, the forward adaptor sequence is SEQ ID NO: 1, and the forward adaptor primer comprises SEQ ID NO: 2. Advantageously, annealing the linear amplification primer to an adaptor sequence rather than the ROI itself allows for more uniform amplification in multiplexed reactions and can reduce the amount of off-target amplification. The optimal length of the forward adaptor primer may be similar to or shorter than the length of primers used for standard PCR, i.e., between about 10 and about 20 nucleotides. In exemplary embodiments, the linear amplification comprises at least 12 PCR cycles. However, the number of cycles may be scaled according to the amount of DNA at the start of the reaction.

In Step (d), the DNA is subjected to exponential amplification using both an exon-specific reverse primer and a reverse adaptor primer. The exon-specific reverse primer is used to generate PCR products from the original linear amplification in combination with the forward adaptor primer from Step (c). The generated PCR products are further amplified by the reverse adaptor primer in combination with the forward adaptor primer to generate enough DNA to be sequenced. In exemplary embodiments, the exponential amplification of Step (d) comprises at least 20 PCR cycles. Additional PCR cycles were needed compared to the prior methods using bacterial templates because the genomic DNA of mammals is larger and there are less copies of the templates within the reaction at the same concentration of genomic DNA.

The exon-specific reverse primer is designed to add a reverse adaptor sequence to the 5′ end of ROI, and it comprises from 5′ to 3′: a reverse adaptor sequence and a second ROI-specific sequence. The adaptor-barcode primer may have varying lengths and compositions as required by the protocol.

The second ROI-specific sequence is a sequence that is identical to the 5′ end of the digested ROI, allowing the exon-specific reverse primer to anneal to the 3′ end of the product from linear amplification of digested ROI. The second ROI-specific sequence may be about 8-30 nucleotides in length.

The reverse adaptor primer is designed to anneal to the reverse adaptor sequence added by the exon-specific reverse primer to amplify the DNA product. For example, in some embodiments, the exon-specific reverse primer comprises the reverse adaptor sequence of SEQ ID NO: 3 and the reverse adaptor primer comprises SEQ ID NO: 4. The optimal length of the reverse adaptor primer may be similar to or shorter than the length of primers used for standard PCR (between about 10 and about 20 nucleotides).

An index sequence may be added to the adaptor-barcode primer and the exon-specific revers primer, but it is not always necessary. On the adaptor-barcode primer, the index sequence (first index sequence) may be between the forward adaptor sequence and the barcode sequence. On the exon-specific reverse primer, the index sequence (second index sequence) may be between the reverse adaptor sequence and the second ROI-specific sequence. Index sequences may be used to increase the total index variations (i.e., increase how many samples you can pack into one sequencing library). They're unique identifying sequences that are used to track DNA in a multiplexed sequencing reaction. In contrast to a barcode sequence, which is usually read in the same read as the genomic DNA, the index sequence is read in a separate sequencing read. Many index sequences are commercially available. In exemplary embodiments, the index sequence is 0-7 nucleotides in length, or 1-7 nucleotides in length. Each of the first and second index sequences may be selected from A, GA, CGA, TCGA, ATCGA, GATCGA, and CGATCGA. The first and second index sequence may be the same or different.

In Step (e) of the present methods, the DNA products are sequenced. Any suitable sequencing method may be used with the present methods. For example, sequencing may be accomplished using a next generation sequencer, such as a NextSeq 550, 10×, Illumina, or another sequencing instrument. Paired-end sequencing may be used to increase the yield of sequencing reads. Single-end sequencing may be used.

The sequencing results are compared to a reference sequence to identify the mutation in the KRAS gene. The term “reference sequence” is used to refer the known normal or wild-type sequence of the KRAS gene of interest. Reference sequences can be obtained, for example from a reference genome, such as those provided by the National Center for Biotechnology Information. A mutation in the KRAS gene is identified when the reference sequence does not match the majority of the sequencing results at a particular position within the ROI. Sequencing data may be analyzed using any known method including, for example, through the Galaxy web platform⁷³.

To detect mutations within the desired regions of interest (ROI) within the human KRAS gene, the inventors selected appropriate enzymes that target a site at the 3′ end of the ROIs and designed ROI-specific primers (i.e., an adaptor-barcode primer and an exon-specific reverse primer) for the amplification of the enzyme digested ROI. Specifically, to detect the transcribed strand of human KRAS exon 1, the inventors used the restriction enzyme StuI to digest the genomic DNA, and they used an adaptor-barcode primer that comprises the first ROI-specific sequence of

SEQ ID NO: 6 and an exon-specific reverse primer comprises the second ROI-specific sequence of SEQ ID NO: 7 or SEQ ID NO: 8. To detect the non-transcribed strand of human KRAS exon 1, the inventors used the restriction enzyme Hinfl to digest the genomic DNA, and they used an adaptor-barcode primer that comprises the first ROI-specific sequence of SEQ ID NO: 9 and an exon-specific reverse primer comprises the second ROI-specific sequence of SEQ ID NO: 10. To detect the non-transcribed strand of human KRAS exon 2, the inventors used the restriction enzyme XmnI to digest the genomic DNA, and they used an adaptor-barcode primer that comprises the first ROI-specific sequence of SEQ ID NO: 11 and an exon-specific reverse primer comprises the second ROI-specific sequence of SEQ ID NO: 12. Thus, any of these sets of restriction enzymes and primers may be used to detect a mutation in an ROI within the human KRAS gene using the methods of the present invention. Other identified sets of restriction enzymes and primers include, but are not limited to: restriction enzyme Hinfl, first ROI-specific sequence SEQ ID NO: 25, second ROI-specific sequence SEQ ID NO: 17; restriction enzyme MluCI, first ROI-specific sequence SEQ ID NO: 26, second ROI-specific sequence SEQ ID NO: 17; restriction enzyme Hpy188I, first ROI-specific sequence SEQ ID NO: 27, second ROI-specific sequence SEQ ID NO: 17; restriction enzyme AlwI, first ROI-specific sequence SEQ ID NO: 28, second ROI-specific sequence SEQ ID NO: 17; restriction enzyme DpnII, first ROI-specific sequence SEQ ID NO: 29, second ROI-specific sequence SEQ ID NO: 17; restriction enzyme MnlI, first ROI-specific sequence SEQ ID NO: 30, second ROI-specific sequence SEQ ID NO: 17; restriction enzyme NsiI, first ROI-specific sequence SEQ ID NO: 31, second ROI-specific sequence SEQ ID NO: 17; restriction enzyme HpyCH4V, first ROI-specific sequence SEQ ID NO: 32, second ROI-specific sequence SEQ ID NO: 17; restriction enzyme BsrI, first ROI-specific sequence SEQ ID NO: 33, second ROI-specific sequence SEQ ID NO: 17; restriction enzyme PsiI, first ROI-specific sequence SEQ ID NO: 18, second ROI-specific sequence SEQ ID NO: 8; restriction enzyme Tsp45I, first ROI-specific sequence SEQ ID NO: 19, second ROI-specific sequence SEQ ID NO: 8; restriction enzyme AflIII, PciI and FatI, first ROI-specific sequence SEQ ID NO: 20, second ROI-specific sequence SEQ ID NO: 8; restriction enzyme NspI and NlaIII, first ROI-specific sequence SEQ ID NO: 21, second ROI-specific sequence SEQ ID NO: 8; restriction enzyme CviAII, first ROI-specific sequence SEQ ID NO: 22, second ROI-specific sequence SEQ ID NO: 8; restriction enzyme CviQ, first ROI-specific sequence SEQ ID NO: 23, second ROI-specific sequence SEQ ID NO: 8; and restriction enzyme HphI, first ROI-specific sequence SEQ ID NO: 24, second ROI-specific sequence SEQ ID NO: 8. The first ROI-specific sequence and second ROI-specific sequence identified in any of the disclosed pairs, may be unpaired and used independently of each other.

The methods may further comprise isolating the genomic DNA prior to the digestion step. Genomic DNA may be isolated from the cells within the biological samples using standard methods that are well known in the art, including those that rely on organic extraction, ethanol precipitation, silica-binding chemistry, cellulose-binding chemistry, and ion exchange chemistry. Many reagents and kits are for DNA isolation are commercially available. In exemplary embodiments, genomic DNA is isolated by phenol/chloroform extraction followed by ethanol precipitation.

Any sample that comprises genomic DNA may be used with the present invention. The term “genomic DNA” refers to the chromosomal DNA of an organism.

About a third of all human cancers are driven by mutations in RAS genes. Thus, in some embodiments, the methods are performed on a sample from a subject that has or is suspected of having cancer. In such cases, the sample may comprise a tumor biopsy, circulating tumor cells (CTCs; i.e., a liquid biopsy), or circulating tumor DNA (ctDNA). As used herein the term “cancer” refers to an abnormal mass of tissue in which the growth of the mass surpasses and is not coordinated with the growth of normal tissue. The term cancer includes both benign and malignant cancers. Typical cancers include but are not limited to carcinomas, lymphomas, or sarcomas, such as, for example, ovarian cancer, colon cancer, breast cancer, pancreatic cancer, lung cancer, prostate cancer, colorectal cancer, endometrial cancer, urinary tract cancer, uterine cancer, acute lymphatic leukemia, Hodgkin's disease, small cell carcinoma of the lung, melanoma, neuroblastoma, glioma, and soft tissue sarcoma of humans, among others.

Because MDS allows for the detection of extremely rare oncogenic mutations, this method is well suited for detecting genetic heterogeneity within a particular subject or within a particular tumor. Thus, in some embodiments, the detected mutation is an oncogenic mutation. In some embodiments, the mutation is an oncogenic driver mutation, i.e., a mutation that is responsible for both the initiation and maintenance of the cancer. Thus, the methods of the present invention can be used in the clinic, for example, to predict cancer treatment outcomes before chemotherapy is administered.

The terms “nucleic acid” “polynucleotide,” and “oligonucleotide,” as used herein, refer to polydeoxyribonucleotides (containing 2-deoxy-D-ribose), polyribonucleotides (containing D-ribose), and to any other type of polynucleotide that is an N glycoside of a purine or pyrimidine base. There is no intended distinction in length between the terms “nucleic acid”, “oligonucleotide” and “polynucleotide”, and these terms will be used interchangeably. These terms refer only to the primary structure of the molecule. Thus, these terms include double- and single-stranded DNA, as well as double- and single-stranded RNA. For use in the present methods, an oligonucleotide also can comprise nucleotide analogs in which the base, sugar, or phosphate backbone is modified as well as non-purine or non-pyrimidine nucleotide analogs.

Oligonucleotides can be prepared by any suitable method, including direct chemical synthesis by a method such as the phosphotriester method of Narang et al., 1979, Meth. Enzymol. 68:90-99; the phosphodiester method of Brown et al., 1979, Meth. Enzymol. 68:109-151; the diethylphosphoramidite method of Beaucage et al., 1981, Tetrahedron Letters 22:1859-1862; and the solid support method of U.S. Pat. No. 4,458,066, each incorporated herein by reference. A review of synthesis methods of conjugates of oligonucleotides and modified nucleotides is provided in Goodchild, 1990, Bioconjugate Chemistry 1(3): 165-187, incorporated herein by reference.

The term “amplification reaction” refers to any chemical reaction, including an enzymatic reaction, which results in increased copies of a template nucleic acid sequence or results in transcription of a template nucleic acid. Amplification reactions include reverse transcription, the polymerase chain reaction (PCR), including Real Time PCR (see U.S. Pat. Nos. 4,683,195 and 4,683,202; PCR Protocols: A Guide to Methods and Applications (Innis et al., eds, 1990)), and the ligase chain reaction (LCR) (see Barany et al., U.S. Pat. No. 5,494,810). Exemplary “amplification reactions conditions” or “amplification conditions” typically comprise either two or three step cycles. Two-step cycles have a high temperature denaturation step followed by a hybridization/elongation (or ligation) step. Three step cycles comprise a denaturation step followed by a hybridization step followed by a separate elongation step. Multiplex polymerase PCR refers to the use of polymerase chain reaction to amplify several different DNA sequences simultaneously (as if performing many separate PCR reactions all together in one reaction). This process amplifies DNA in samples using multiple primers and a temperature-mediated DNA polymerase in a thermal cycler. The primer design for all primers pairs has to be optimized so that all primer pairs can work at the same annealing temperature during PCR.

The terms “target,” “target sequence”, “target region”, and “target nucleic acid,” as used herein, are synonymous and refer to a region or sequence of a nucleic acid which is to be amplified, sequenced, or detected.

The terms “annealing” and “hybridization,” as used herein, refer to the formation of a duplex structure by two single-stranded nucleic acids due to complementary base pairing. Hybridization can occur between fully complementary nucleic acid strands or between “substantially complementary” nucleic acid strands that contain minor regions of mismatch. Conditions under which hybridization of fully complementary nucleic acid strands is strongly preferred are referred to as “stringent hybridization conditions” or “sequence-specific hybridization conditions”. Stable duplexes of substantially complementary sequences can be achieved under less stringent hybridization conditions; the degree of mismatch tolerated can be controlled by suitable adjustment of the hybridization conditions. Those skilled in the art of nucleic acid technology can determine duplex stability empirically considering a number of variables including, for example, the length and base pair composition of the oligonucleotides, ionic strength, and incidence of mismatched base pairs, following the guidance provided by the art (see, e.g., Sambrook et al., 1989, Molecular Cloning—A Laboratory Manual, Cold Spring Harbor Laboratory, Cold Spring Harbor, New York; Wetmur, 1991, Critical Review in Biochem. and Mol. Biol. 26(3/4):227-259; and Owczarzy et al., 2008, Biochemistry, 47: 5336-5353, which are incorporated herein by reference).

The term “primer,” as used herein, refers to an oligonucleotide capable of acting as a point of initiation of DNA synthesis under suitable conditions. Such conditions include those in which synthesis of a primer extension product complementary to a nucleic acid strand is induced in the presence of four different nucleoside triphosphates and an agent for extension (for example, a DNA polymerase or reverse transcriptase) in an appropriate buffer and at a suitable temperature. A primer is preferably a single-stranded DNA. The appropriate length of a primer depends on the intended use of the primer but typically ranges from about 6 to about 225 nucleotides, including intermediate ranges, such as from 15 to 35 nucleotides, from 18 to 75 nucleotides and from 25 to 150 nucleotides. Short primer molecules generally require cooler temperatures to form sufficiently stable hybrid complexes with the template. A primer need not reflect the exact sequence of the template nucleic acid but must be sufficiently complementary to hybridize with the template. The design of suitable primers for the amplification of a given target sequence is well known in the art and described in the literature cited herein.

Primers can incorporate additional features which allow for the detection or immobilization of the primer but do not alter the basic property of the primer, that of acting as a point of initiation of DNA synthesis. For example, primers may contain an additional nucleic acid sequence at the 5′ end which does not hybridize to the target nucleic acid, but which facilitates cloning or detection of the amplified product, or which enables transcription of RNA (for example, by inclusion of a promoter) or translation of protein (for example, by inclusion of a 5′ -UTR, such as an Internal Ribosome Entry Site (IRES) or a 3′ -UTR element, such as a poly(A)n sequence, where n is in the range from about 20 to about 200). The region of the primer that is sufficiently complementary to the template to hybridize is referred to herein as the hybridizing region.

As used herein, a primer is “specific,” for a target sequence if, when used in an amplification reaction under sufficiently stringent conditions, the primer hybridizes primarily to the target nucleic acid. Typically, a primer is specific for a target sequence if the primer-target duplex stability is greater than the stability of a duplex formed between the primer and any other sequence found in the sample. One of skill in the art will recognize that various factors, such as salt conditions as well as base composition of the primer and the location of the mismatches, will affect the specificity of the primer, and that routine experimental confirmation of the primer specificity will be needed in many cases. Hybridization conditions can be chosen under which the primer can form stable duplexes only with a target sequence. Thus, the use of target-specific primers under suitably stringent amplification conditions enables the selective amplification of those target sequences that contain the target primer binding sites.

### Kits

In another aspect, provided herein is a kit for detecting a mutation in a KRAS gene in a subject. The kits comprise at least one set of primers for the detection of a mutation in a specific gene region of interest (ROI). The set of primers includes a forward adaptor primer, a reverse adaptor primer, an adaptor-barcode primer, and an exon-specific reverse primer. The set of primers may comprise a forward adaptor primer comprising SEQ ID NO: 2; a reverse adaptor primer comprising SEQ ID NO: 4; an adaptor barcode primer selected from SEQ ID NOs: 34, 37, 39, 41-111, and 119-181; and an exon-specific reverse primer selected from SEQ ID NOs: 35, 38, 40, 112-118, and 182-188. Each of the at least one set of primers should comprise an adaptor barcode primer and an exon-specific barcode primer pair that targets a region of interest in KRAS.

The kit may further comprise at least one enzyme selected from StuI, HinfI, AlwI, BsrI, DpnII, Hpy188I, HpyCH4V, MluCI, MnlI, NsiI, StuI, AflIII, PciI, FatI, NlaIII, CviAII, CviQI, HphI, NspI, PsiI, XmnI, and Tsp45I. The at least one enzyme should cleave KRAS at the 3′ end of the region of interest target by the at least one adaptor barcode primer and the exon-specific barcode primer in the kit.

The kit may further comprise enzymes, buffers, and reagents necessary to perform PCR reactions.

### Miscellaneous

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

“Percentage of sequence identity”, “percent similarity”, or “percent identity” is determined by comparing two optimally aligned sequences over a comparison window, wherein the portion of the polynucleotide or peptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” or “substantial similarity” of polynucleotide or peptide sequences means that a polynucleotide or peptide comprises a sequence that has at least 75% sequence identity. Alternatively, percent identity can be any integer from 75% to 100%. More preferred embodiments include at least: 75%, 80%, 85%, 86%, 87%, 88%, 89%, 90%, 91%, 92%, 93%, 94%, 95%, 96%, 97%, 98% or 99% compared to a reference sequence using the programs described herein; preferably BLAST using standard parameters, as described. These values can be appropriately adjusted to determine corresponding identity of proteins encoded by two nucleotide sequences by taking into account codon degeneracy, amino acid similarity, reading frame positioning and the like. Any of the primers sequences disclosed in include sequences having at least 75%, 80%, 85%, 86%, 87%, 88%, 89%, 90%, 91%, 92%, 93%, 94%, 95%, 96%, 97%, 98% or 99% identity thereto.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein. Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

### Example 1: Demonstrating High Sensitivity Method of Detecting Mutations in Ras Gene in a Mammalian Model

The environmental carcinogen urethane exhibits a profound specificity for pulmonary tumors driven by an oncogenic Q₆₁L/R mutation in the gene Kras. Similarly, the frequency, isoform, position, and substitution of oncogenic RAS mutations are often unique to human cancers. In the following example, to elucidate the principles underlying this RAS mutation tropism caused by urethane, the inventors adapted an error-corrected, high-throughput sequencing approach to detect mutations in murine Ras genes with high sensitivity. This approach not only captured the initiating Kras mutation days after urethane exposure, but also revealed that the sequence specificity of urethane mutagenesis coupled with transcription and isoform locus are major influences on the extreme tropism of this carcinogen.

### Materials and Methods

Cell culture. Mouse embryonic fibroblasts (MEFs) derived from E13.5 mouse embryos were stably infected with an ecotropic retrovirus derived from pBabeHygro⁷⁰ encoding the early region of SV40⁷¹ and selected with 100 μg·ml⁻¹ hygromycin to establish immortalized cultures using standard procedures and then cultured in Dulbecco's modified Eagle's medium (DMEM) supplemented with 10% fetal bovine serum (FBS) and 1% penicillin-streptomycin.

Construction of Kras mutant plasmids. A region upstream of Kras start codon was amplified from murine genomic DNA (termed PCR1). PCR reactions were comprised of 100 ng of genomic DNA, 2.5 μl of 10 μM forward (5′-AATTGCGGCCGCCCAGGGGGTATAGCGTACTATGCAGAAT-3′) (SEQ ID NO: 189) and reverse (5′-CATTTTCAGCAGGCCTTACAAT-3′) (SEQ ID NO: 190) primers, 4 μl of 2.5 mM dNTP, 10 μl of 5×buffer (NEB), and 0.5 μl Q5 ® Hot Start High-Fidelity DNA Polymerase (NEB) in a total volume of 50 PCR cycles were as follows: one cycle at 98° C. for 30 seconds, 28 cycles at 98° C. for 8 seconds, 64° C. for 15 seconds, 72° C. for 10 seconds, and one cycle at 72° C. for 2 minutes. PCR products were gel purified using QIAquick Gel Extraction Kit following the manufacturer's protocol (Qiagen).

Mutations in Kras cDNA were generated through error-prone PCR (termed PCR2). PCR reactions were comprised of 15 nmol of plasmid containing Kras cDNA, 2 μl of 10 μM forward (5′ -AT TGTAAGGCC TGC TGAAAGAAGAGTATAAACT TGTGGT-3 ‘) (SEQ ID NO: 191) and reverse (5’-CAGGGTCGACTCACATAACTGTACACCTTGTC-3′) (SEQ ID NO: 192) primers, 2 μl of 2.5 mM dNTP, 1.25 μl of 50 mM MgCl₂, 2.5 μl of 10×buffer (Invitrogen), 5 μl of 2.5 mM MnCl₂, and 0.2 μl of Platinum Taq DNA polymerase (Invitrogen) in a total volume of 25 PCR cycles were as follows: one cycle at 94° C. for 1 minute, 18 cycles at 94° C. for 30 seconds, 55° C. for 30 seconds, 72° C. for 3 minutes, and one cycle at 72° C. for 3 minutes. PCR products were gel purified as described above.

Products from PCR1 and PCR2 were fused through overlap PCR (termed PCR3). 20 ng product from PCR1 and 40 ng product from PCR2 were mixed with 4 μl of 2.5 mM dNTP, 10 μl of 5×buffer (NEB), and 0.5 μl Q5® Hot Start High-Fidelity DNA Polymerase (NEB) in a total volume of 50 μl reaction. PCR cycles were 98° C. for 30 seconds and 10 cycles at 98° C. for 8 seconds, 63° C. for 15 seconds, and 72° C. for 15 seconds. 2.5 μl of forward primer from PCR1 and 2.5 μl of reverse primer from PCR2 were then added and the reaction was continued in the following conditions: 98° C. for 30 seconds, 25 cycles at 98° C. for 8 seconds, 72° C. for 40 seconds, and one cycle at 72° C. for 2 minutes. PCR products were gel purified as described above.

Plasmid backbone was amplified from the pUC19⁷² (Addgene 50005) plasmid (termed PCR4). PCR reactions were comprised of 1 ng of pUC19 DNA, 2.5 μl of 10 μM forward (5′-AATTGTCGACTTAGACGTCAGGTGGCAC-3′) (SEQ ID NO: 193) and reverse (5′-TTAAGCGGCCGCGTTTGCGTATTGGGCGCT-3′) (SEQ ID NO: 194) primers, 4 μl of 2.5 mM dNTP, 10 μl of 5×buffer (NEB), and 0.5 μl Q5° Hot Start High-Fidelity DNA Polymerase (NEB) in a total volume of 50 PCR cycles were as follows: one cycle at 98° C. for 30 seconds, 28 cycles at 98° C. for 8 seconds, 65° C. for 15 seconds, 72° C. for 1 minute, and one cycle at 72° C. for 2 minutes. PCR products were gel purified as described above.

Products from PCR3 and PCR4 were digested with Sall and Notl according to the manufacture's protocol (NEB). Digested products were column purified using QIAquick PCR Purification Kit following the manufacturer's protocol (Qiagen), ligated, and transformed using standard methodologies. DNA was isolated from individual clones by NucleoSpin® Plasmid miniprep kit (MACHEREY-NAGEL) and validated by Sanger sequencing. Ten clones with different sets of co-occurring mutations in Kras exon 1 and/or 2 were selected to be spiked into wildtype mouse genomic DNA at different ratios to test the detection limit of maximum depth sequencing (see below).

Urethane treatment. 6-8-week-old male and female A/J mice (JAX Stock #000646) were intraperitoneally injected daily for three days with either urethane (Sigma U2500) dissolved in PBS (1 g·kg⁻¹) or the vehicle PBS alone. Mice were humanely euthanized 1, 2, 3, or 4 weeks after the last injection and the lung, liver, and pancreas collected for the extraction of genomic DNA. All mouse care and experiments were performed in accordance with protocols approved by the IACUC of Duke University.

Pharmacokinetic analysis. 6-8-week-old male and female A/J mice (JAX Stock #000646) were intraperitoneally injected with one dose of urethane dissolved in PBS (1 g·kg⁻¹). Mice were humanely euthanized 2, 4, and 8 hours later after which plasma, lungs, pancreas, and livers were harvested and snap frozen. Liquid chromatography (LC) tandem-mass spectrometry (MS/MS) was used to measure urethane (ethyl carbamate, EC) (Sigma U2500) and vinyl carbamate (VC) (Santa Cruz Biotechnology sc-213157) concentrations in plasma and tissues. The LC-MS/MS system consisted of Shimadzu 20A series LC and Applied Biosystems/SCIEX API 4000 QTrap MS/MS instrument. LC columns: Phenomenex C₁₈ 3×4 mm guard column (#AJO-4287) and Agilent ZORBAX Eclipse Plus C₁₈ 150×4.6 mm 1.8 μm analytical column (#959994-902). Mobile phase A: 0.1% formic acid, 10 μM sodium acetate, and 2% acetonitrile; mobile phase B: 100% methanol. Elution gradient: isocratic flow 30% A. Flow rate: 0.8 ml·min 1. The run time was 10 minutes. Calibration samples were prepared by adding pure standards of EC or VC to corresponding matrix (plasma or tissue homogenate) in appropriate concentration range. The calibration samples were analyzed alongside study samples as a single analytical batch on the day of analysis.

In 2 ml screw cap vial, 20 μl (EC) or 50 μl (VC) of plasma or tissue homogenate (1 part tissue and 2 parts water) diluted with water 1/100 (EC) or undiluted (VC), 10 μl of 2 μg·ml ⁻¹ MC-d5 in water (internal standard) (Toronto Research Chemicals), and 60 μl (EC) or 100 μl (VC) of 20 mM xanthydrol (Sigma) in glacial HAc were added and incubated at room temperature for 30 minutes. 100 μl of water and 500 μl of chloroform were then added and the mixture was vigorously agitated (speed 4, 40 seconds; Fast-Prep FP120, Thermo Savant). After centrifugation at 16,000 g for 5 minutes at room temperature, 200 μl (EC) or 400 μl (VC) of chloroform (lower) layer was subjected to a gentle stream of nitrogen for 30 minutes, dry residue reconstituted with 50 μl (EC) or 100 μl (VC) 50% A/50% B, centrifuged at 16,000 g for 5 minutes at 4° C., after which 5 μl (EC) or 10 μl (VC) was injected into LC-MS/MS system. The mass spectrometer was operated in positive mode with the following MRM transitions (m/z): 292/180.8 [EC-1st], 292/151.3 [EC-2nd], 297/181.8 [EC-d5-1st], 297/151.7 [EC-d5-2nd] for EC and 290/180.5 [VC-1st], 290/151.2 [VC-2nd], 297/181.8 [EC-d5-1st], 297/151.7 [EC-d5-2nd] for VC.

Isolation of genomic DNA. MEF cells were resuspended in lysis buffer (100 mM NaCl, 10 mM Tris pH 7.6, 25 mM EDTA pH 8.0, and 0.5% SDS in H₂O, supplemented with 20 μg·ml⁻¹ RNase A (Sigma)). Lung, pancreas, and liver (right lobe) from A/J mice (JAX Stock #000646) were cut into fine pieces and similarly resuspended in lysis buffer. Samples were incubated at 37° C. for 1 hour. 2 μl of 800 U·ml⁻¹ proteinase K (NEB) was then added to each sample, the samples were vortexed, and then incubated at 55° C. overnight. Genomic DNA was isolated by phenol/chloroform extraction followed by ethanol precipitation using standard procedures and quantified using Qubit fluorometer.

Maximum depth sequencing (MDS). The MDS assay¹³ was adapted for mammalian Ras genes as follows. 20-50 μg of genomic DNA was incubated with StuI (NEB) for analysis of the transcribed strand of Kras exon 1, EcoRV (NEB) and EcoRI (NEB) for analysis of the non-transcribed strand of Kras exon 1, XmnI (NEB) for analysis of the non-transcribed strand of Kras exon 2, and PleI (NEB) for analysis of the transcribed strand of Kras exon 2, or HphI (NEB) for the analysis of the non-transcribed strand of Hras exon 2. Reaction conditions were 5 units of the indicated restriction enzyme and per 1 μg DNA per 20 μl reaction (e.g., 20 μg genomic DNA, 5 μl enzyme (20 units/μl), and 40 μl 10×buffer in 400 μl reaction). Digested genomic DNA was column purified using QIAquick PCR Purification Kit following the manufacturer's protocol (Qiagen) and resuspended in ddH₂O (35 μl H₂O per 10 μg DNA). The barcode and adaptor were added to the target DNA by incubating purified DNA with the appropriate barcode primer (see below) for one cycle of PCR. PCR reactions were comprised of 10 μg DNA, 2.5 μl of 10 μM barcode primer, 4 μl of 2.5 mM dNTP, 10 μl of 5×buffer (NEB), and 0.5 μl Q5® Hot Start High-Fidelity DNA Polymerase (NEB) in a total volume of 50 μl. The number of PCR reactions was scaled according to the amount of DNA. PCR conditions were 98° C. for 1 minute, barcode primer annealing temperate (see below) for 15 seconds, and 72° C. for 1 minute. 1 μl of 20,000 U.m1 1 exonuclease I (NEB) and 5 μl of 10×exonuclease I buffer (NEB) was then added to each 50 μl reaction to remove unused barcoded primers and incubated at 37° C. for 1 hour and then 80° C. for minutes. Processed DNA were column purified using QIAquick PCR Purification Kit as above and resuspended in ddH₂O (35 μl H₂ per column). The concentration of purified product was measured with SimpliNano spectrophotometer (GE Healthcare Life Sciences). Samples were linear amplified with forward adaptor primer (see below). PCR reactions were comprised of 1.5 μg DNA, 2.5 μl of 10 μM forward-adaptor primer, 4 μl of 2.5 mM dNTP, 10 μl of 5×buffer (NEB), and 0.5 μl Q5® Hot Start High-Fidelity DNA Polymerase (NEB) in a total volume of 50 μl. The number of PCR reactions was scaled according to the amount of DNA. PCR conditions were as follows: 12 cycles of 98° C. for 15 seconds, 70° C. for 15 seconds, 72° C. for 8 seconds. 2.5 μl of 10 μM exon-specific reverse primer (see below) and 2.5 μl of 10 μM reverse-adaptor primer (see below) were then added to each 50 μl reaction. The mixtures were then subjected to 20 cycles of exponential amplification. PCR conditions were as follows: 4 cycles of 98° C. for 15 seconds, exon-specific reverse primer annealing temperature (see below) for 15 seconds, 72° C. for 8 seconds, 16 cycles of 98° C. for 15 seconds, 70° C. for 15 seconds, and 72° C. for 8 seconds. The final library was size selected and purified with Ampure XP beads according to the manufacturer's protocol (Beckman Coulter). Sequencing was performed using HiSeq 2500 100 bp PE rapid run, HiSeq 4000 150 bp PE or NovaSeq 6000 S Prime 150 bp PE at Duke Center for Genomic and Computational Biology. For the optimization of barcode recovery, the same amount of genomic DNA was processed in parallel by MDS assay targeting Kras exon 1 transcribed strand and the PCR products were pooled together at different concentrations in one library to obtain different sequencing depths.

### Primers for Maximum Depth Sequencing

Barcode primer: [Forward adaptor][Index]
[Barcode][ROI-specific Primer][Forward
adaptor] =
(SEQ ID NO: 1)
5′-TACGGCGACCACCGAGATCTACACTCTTTCCCTACACGACGCTCTTC
CGATCT-3′
[Index] = variable length of known sequences
from 0 to 7 nucleotides (Table 1) [Barcode] =
NNNNNNNNNNNNNN [ROI-specific Primer] = Table 2

TABLE 1
[Index] Sequences

| Index Sequence # | Sequence (5′→3′) |
|---|---|
| 1 | A |
| 2 | GA |
| 3 | CGA |
| 4 | TCGA |
| 5 | ATCGA |
| 6 | GATCGA |
| 7 | CGATCGA |

TABLE 2
[ROI-specific Primers] of Barcode primer

| | SEQ | | Annealing |
|---|---|---|---|
| | ID | | Temp. |
| Primer | NO: | Sequence (5′→3′) | (° C.) |
| Kras exon 1 | 195 | CCTGCTGAAAATGACTGAG | 60 |
| StuI | | | |
| Kras exon 1 | 196 | ATCTTTTTCAAAGCGGCTG | 68 |
| EcoRV | | GCT | |
| Kras exon 2 | 11 | TCTTCAAATGATTTAGTAT | 59 |
| XmnI | | TATTTATGGC | |
| Kras exon 2 | 197 | TCAGGACTCCTACAGGAAAC | 63 |
| PleI | | | |
| Hras exon 2 | 198 | TAGGTGGCTCACCTGTACTG | 66 |
| HphI | | | |

Forward-adaptor primer:
(SEQ ID NO: 2)
5′-AATGATACGGCGACCACCGAGAT-3′
(annealing temperature: 70° C.)
Exon-specific reverse primer: [Reverse
adaptor][Index][ROI-specific Primer]
[Reverse adaptor] =
(SEQ ID NO: 199)
5′-AAGCAGAAGACGGCATACGAGATCGGTCTCGGCATTCCTGCTGAACC
GCTCTTCCGATCT-3′
(for barcode recovery optimization and
Kras exon 2 mutant plasmid spike in
experiment)
or
(SEQ ID NO: 3)
5′-CAAGCAGAAGACGGCATACGAGATGTGACTGGAGTTCAGACGTGTGC
TCTTCCGATCT-3′
(for all the other experiments) [Index] = variable
length of known sequences from 0 to 7 nucleotides
(Table 1) [Primer] = Table 3

TABLE 3
[ROI-specific Primers] of Exon-specific
reverse primer

| | SEQ | | Annealing |
|---|---|---|---|
| | ID | | Temp. |
| Primer | NO: | Sequence (5′→3′) | (° C.) |
| Kras exon 1 StuI | 200 | CTCTATCGTAGGGTCA | 62 |
| | | TACTCAT | |
| Kras exon 1 | 201 | TATTATTTTTATTGTA | 62 |
| EcoRV | | AGGCCTGCTGA | |
| Kras exon 2 XmnI | 202 | GACTCCTACAGGAAAC | 62 |
| | | AAGT | |
| Kras exon 2 PleI | 203 | CTTTCTTATTCAACTT | 59 |
| | | AAACCCAC | |
| Hras exon 2 HphI | 204 | CTAAGCCGTGTTGTTT | 65 |
| | | TGCAG | |

| | Reverse-adaptor primer: |
|---|---|
| (SEQ ID NO: 4) | |
| | 5′-CAAGCAGAAGACGGCATACGAGA-3′ |
| | (annealing temperature: 70° C.) |

All primers were synthesized by Integrated DNA Technologies (IDT).

Data analysis. All sequencing data were analyzed through the Galaxy web platform⁷³. Specifically, raw data were uploaded to the usegalaxy website or Galaxy Cloudman. For analysis of Kras exon 1 transcribed strand in mouse lung tissue, only read 1 was used. For all the other experiments, read 1 and read 2 were joined via PEAR pair-end read merger⁷⁴. The reads were then filtered by quality by requiring 90% of bases in the sequence to have a quality core ≥20. Filtered reads were split into different files based on assigned sample indexes and variation in sequence lengths using the tool Barcode Splitter and the tool Filter Sequences by Length.

For the experiment optimizing barcode recovery, the reads were trimmed down to the barcode and grouped into families by barcode. The number of families containing 1 read and ≥2 reads was then counted, respectively.

For the mutant plasmid spike-in experiments, the reads were trimmed down to the barcode and the bases containing engineered mutations. Trimmed reads were grouped by barcode into different families. The frequency of mutants present was calculated by dividing the counts of families containing engineered co-occurring mutations by the total number of families. The frequency of mutants detected was calculated by dividing the counts of families containing ≥2 reads and have ≥90% reads sharing the same engineered mutation at one specified position by the total number of families.

For the experiments examining carcinogen-induced mutations in Kras exon 1 or 2, the reads were trimmed down to the barcode and the target exon. Trimmed reads were grouped by barcode. Barcode families containing ≥3 reads and a unique consensus sequence were selected. To ensure sufficient barcode recovery for the purpose of sensitivity and accuracy, samples with less than 1.5×10⁵ barcode families recovered were excluded from downstream analysis. Sequences from selected barcode families were compared against annotated reference mutant sequences containing all possible single nucleotide substitutions in the exon of interest and the mutation in the reference mutant sequence was assigned to the matched barcode family. The frequency of the corresponding mutation was calculated by dividing the counts of the families containing the mutation by the total number of families.

C>T and G>T substitutions have high background in PBS-treated mouse and have been previously identified as artifacts caused by deamination of cytosine or methyl-cytosine or oxidation of guanine arising during in library preparation^{11,75}, or mis-incorporated nucleotides in vivo not yet repaired¹³. Consistent with this, we detect high C>T or G>T substitutions but not the complementary G>A or C>A substitutions from the strand processed by MDS. To circumvent this background, the frequency of C>T or G>T substitutions was estimated from the strand with the reverse complementary G>A or C>A substitutions when necessary. Specifically, frequency of G_{12/13}C and G_{12/13}V mutations in Kras exon 1 (G>T substitution on the non-transcribed strand) were estimated from the MDS targeting the transcribed strand while frequency of G_{12/13}S and G_{12/13}D mutations in Kras exon 1 (C>T substitutions on the transcribed strand) were estimated from MDS targeting the non-transcribed strand.

Droplet digital PCR (ddPCR). ddPCR was performed using the QX200 AutoDG Droplet Digital PCR System (Bio-Rad) following the manufacturer's protocol in a 22 μl ddPCR reaction containing 11 μl of 2×ddPCR SuperMix for probes (no dUTP) (Bio-Rad), 66 ng template DNA, 450 nM forward and reverse primers, and 250 nM FAM- and HEX-labelled probes. The primer and probe oligonucleotides were synthesized (IDT) based on sequences previously described⁷⁶ with minor modifications. The sequences for the primers are: Kras_Q61_For: 5′-ATGGAGAAACCTGTCTCTTGG-3′ (SEQ ID NO: 205); and Kras_Q61_Rev: 5′-CTCATGTACTGGTCCCTCATT-3′ (SEQ ID NO: 206). The sequences for the probes are: Kras_Q61L_MUT_FAM: 5′ -/56-FAM/CAGGT+C+T+AGA+GGAG/3IABkFQ/-3′; and Kras_Q61L_WT_HEX: 5′ -/5HEX/CAGGT +C+A+AGA+GGAG/3IABkFQ/-3′ where “+” denotes the following base is a locked nucleic acid. Following droplet generation on the AutoDG, the plate was sealed with pierceable foil heat seal (Bio-Rad) and PCR performed on a C1000 Touch™ thermal cycler (Bio-Rad). Thermal cycling conditions were as follows: once cycle at 95° C. for 10 minutes, 40 cycles at 94° C. for 30 seconds and 60° C. for 60 seconds, once cycle at 98° C. for 10 minutes, and 4° C. until the sample was removed. Every ddPCR run included no template control, wildtype control with DNA from PBS-treated mice, and mutation-positive control. To achieve detection sensitivity of 1 in 10,000, each sample was assayed in at least 2 wells. Plates were read on a QX200 droplet reader (Bio-Rad) and analyzed with QuantaSoft™ Analysis Pro software (version 1.0.596) (Bio-Rad) to assess the number of droplets positive for mutant DNA, wild-type DNA, both, or neither. The mutant allele fraction⁴³ was estimated as follows: The concentration of mutant DNA (copies of mutant DNA per droplet) was estimated from the Poisson distribution using the formula number of mutant copies per droplet Mmu=−ln (1−(nmu/n)), where nmu=number of droplets positive for mutant FAM probe and n=total number of droplets. The DNA concentration in the reaction was estimated using the formula MDNAconc=−ln (1−(nDNAconc/n)), where nDNAconc=number of droplets positive for mutant FAM probe and/or wild-type HEX probe and n=total number of droplets. The mutant allele fraction=Mmu/MDNAconc.

RNA isolation and quantitative PCR. RNA was extracted from the lung, liver, and pancreas of 6-week-old A/J mice using TRIzol (Thermo Fisher Scientific) and converted to cDNA using iScript™ cDNA Synthesis Kit (Bio-Rad) following the manufacturer's instructions. Quantitative PCR reactions were performed using iTaq Universal SYBR Green Supermix (Bio-Rad) and CFX384 touch real-time PCR detection system (Bio-Rad) using the forward (5′-CCAGCGTCGTGATTAGCGA-3′ (SEQ ID NO: 207) and reverse (5′-CCAGCAGGTCAGCAAAGAAC-3′) (SEQ ID NO: 208) primers (IDT) to detect the control Hprt mRNA and the forward (5′ -GCAAGAGCGCCTTGACGATA-3′) (SEQ ID NO: 209) and reverse (5′-CATGTACTGGTCCCTCATTGCAC-3′) (SEQ ID NO: 210) primers (IDT) to detect Kras mRNA. Gene expression values were calculated using the comparative Ct (−ΔΔCt) method⁷⁷, using Hprt housekeeping gene as internal control.

Whole exome analysis of mutation frequency versus gene expression. Mutation counts were obtained from published datasets³. Single-nucleotide variations (SNVs) identified by the whole-exome sequencing of urethane-induced adenomas and adenocarcinomas were examined. The expression level of the genes containing these SNVs were determined from published datasets³⁶ . FPKM values of genes expressed in the lung of six-weeks-old C57BL/6JJcl mice were used. The second set of gene expression data 37 were obtained from mouse ENCODE project. FPKM values of genes expressed in the lung of eight-week-old male C57B1/6 mice were used. To bin the genes into different expression groups, the genes were sorted by the mean FPKM value across biological replicates and split into quartiles. The sum of CAN→CTN transversions in the non-transcribed or transcribed strand for the genes in each quartile was calculated and the mean±SEM of all tumors was plotted.

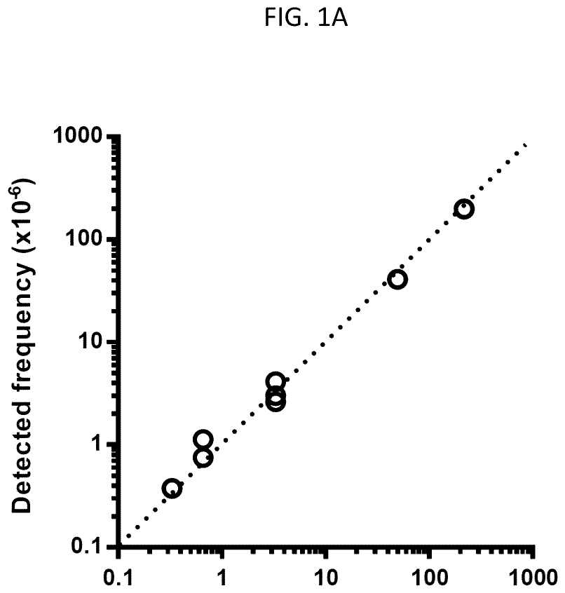

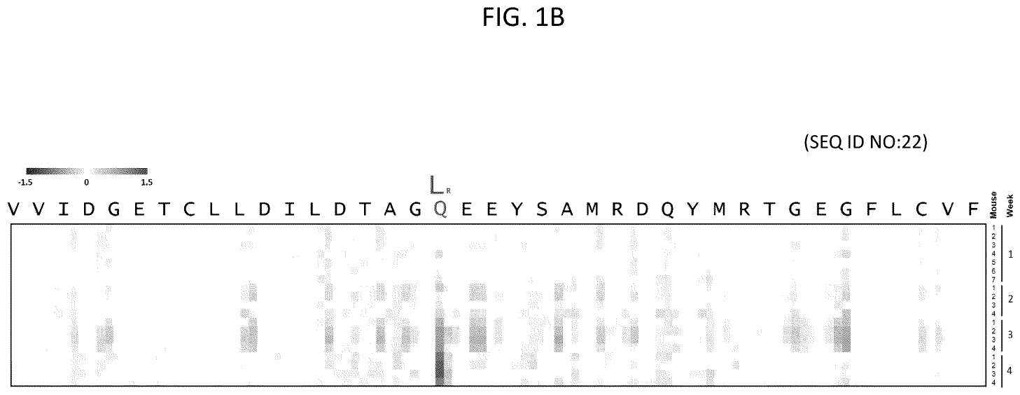

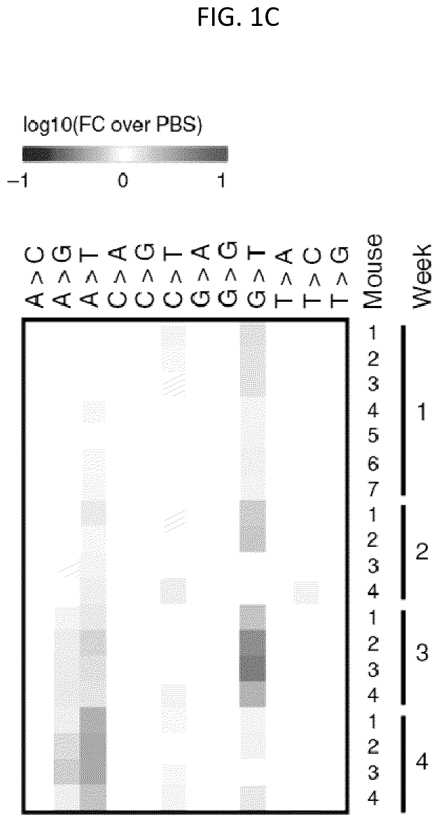

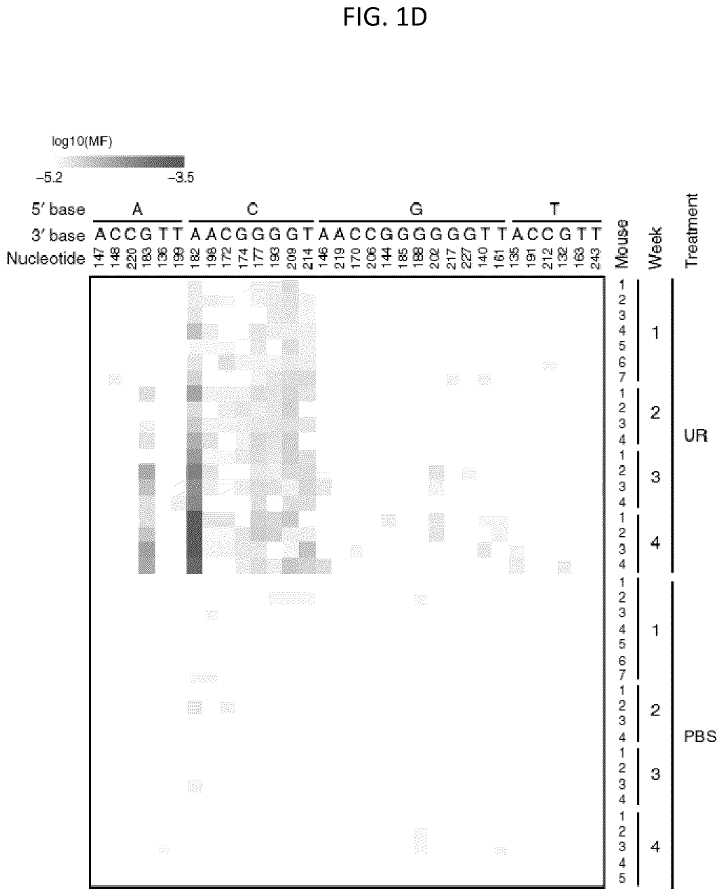

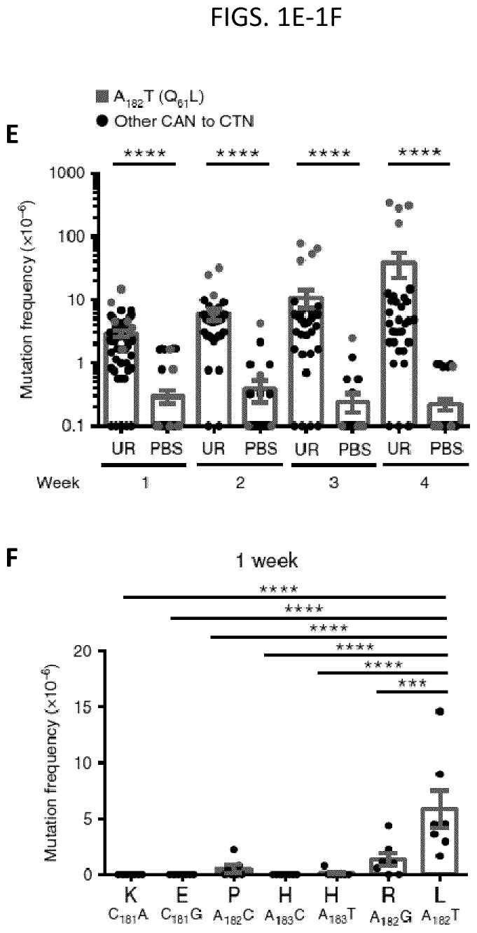

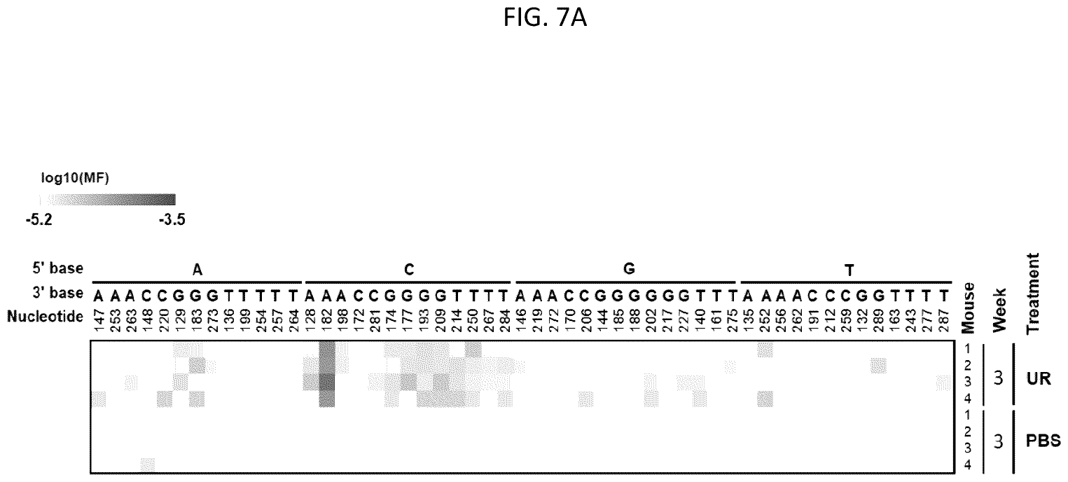

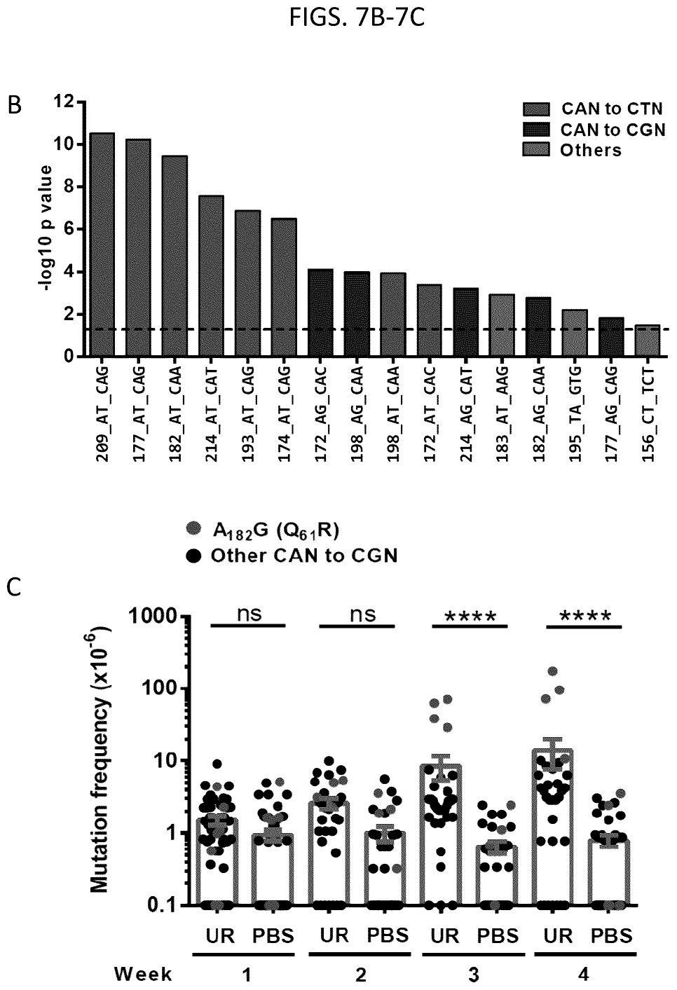

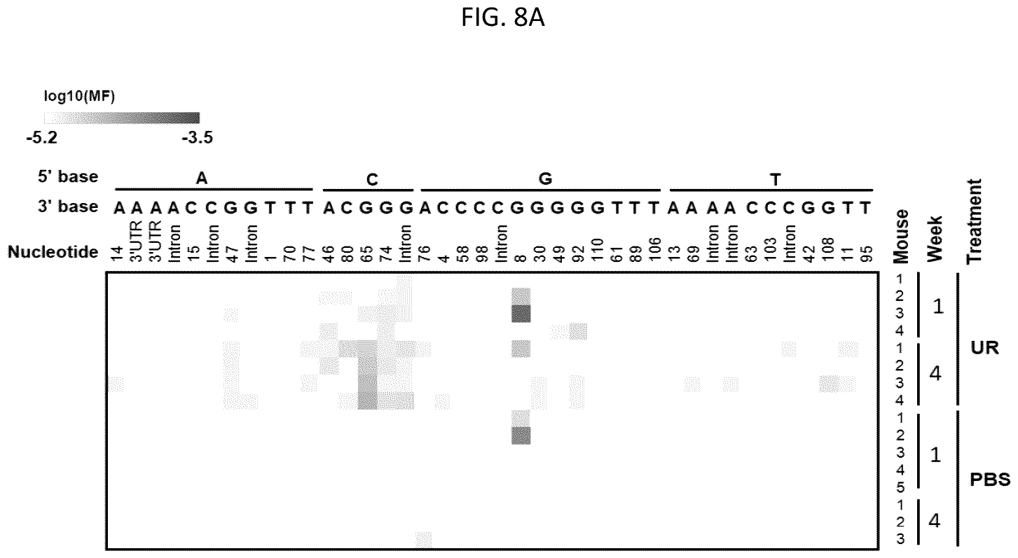

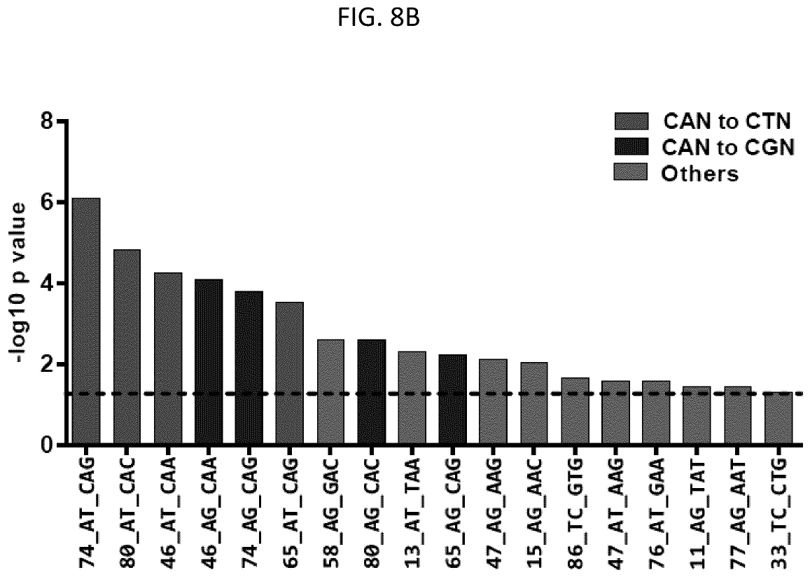

Generation of heatmaps. All heatmaps were generated using Morpheus. All mutation frequencies used in heatmap were corrected by the addition of the detection limit at a barcode recovery of 1.5×10⁵ (˜6.67×10⁻⁶). For the heatmap showing the mutation frequency per nucleotide (FIG. 1b), the sum of the corrected mutation frequencies for all substitutions at an individual nucleotide was obtained, then the fold change of each urethane-treated sample versus the average of PBS-treated samples was calculated and log₁₀ transformed for plotting. For the heatmaps showing the mutation frequency of each type of substitution (FIGS. 1c, 2a, and 3a), the sum of the corrected mutation frequencies for each type of substitutions was obtained, then the fold change of each urethane-treated sample versus the average of PBS-treated samples was calculated and log₁₀ transformed for plotting. For the heatmaps showing the mutation frequency of A>T transversions (FIGS. 1d, 2b, and 3b; FIGS. 7a and 8a), the corrected mutation frequency for each A>T transversion was log₁₀ transformed and plotted.

Statistics. The number of independent experiments and the statistical analysis used are indicated in the legends of each figure. Data are represented as mean±SEM. p values were determined by Holm-Sidak multiple comparisons test following one-way or two-way ANOVA, non-parametric Dunn's multiple comparison test following Kruskal-Wallis test, or two-tailed non-parametric Mann-Whitney U test. p<0.05 was considered significant. Different levels of significance are indicated as *p<0.05, **p<0.01, ***p<0.001, ****p<0.0001 and ns: not significant. Holm-Sidak multiple comparisons test following ANOVA and non-parametric Dunn's multiple comparison test following Kruskal-Wallis test were executed using GraphPad Prism 6. Two-tailed non-parametric Mann-Whitney U test was executed using excel supplemented with Real Statistics Resource Pack.

Data availability. All raw Illumina® sequencing data has been deposited to NCBI Sequence Read Archive (SRA) under accession number PRJNA561927.

### Results

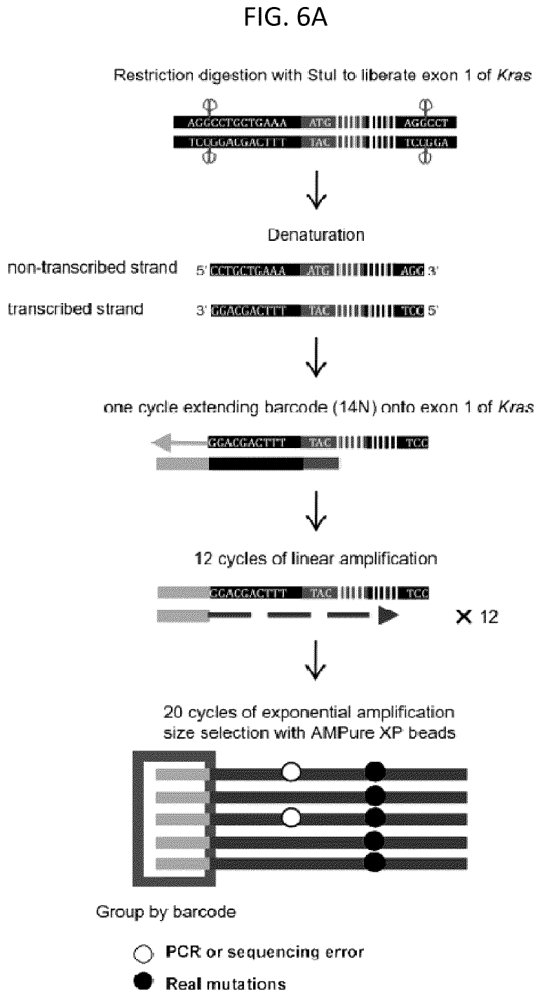

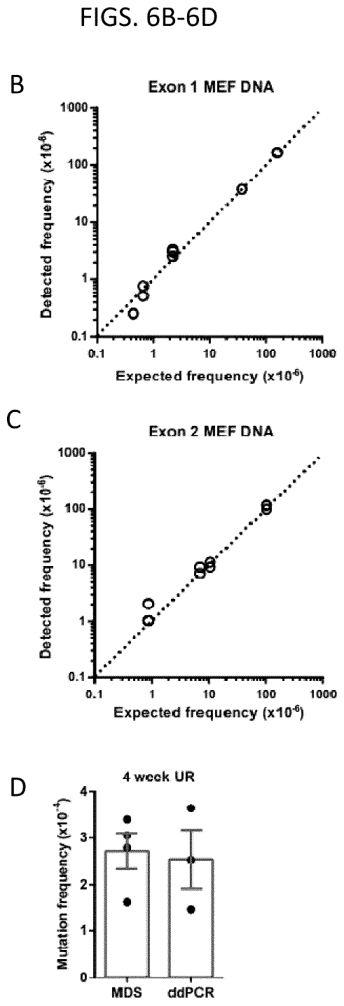

Adapting MDS to the mammalian genome. After urethane exposure, a barrier to detecting initiating Kras mutations in vivo at the time they occur is that the mutation rate of this carcinogen is well below the detection limit of next generation sequencing (NGS). To overcome this limitation, we turned to the error-corrected, high-throughput sequencing approach of maximum depth sequencing (MDS), which is shown to recover mutants in bacteria at a frequency as low as 1×10⁻⁶ or 1 mutant per 10⁶ templates¹³. The key steps of MDS are: first, synthesis of unique barcodes onto one strand of a genomic region-of-interest (ROI); second, linear amplification to obtain multiple direct copies of the barcoded genomic DNA; third, exponential amplification to obtain families of PCR products sharing the same barcode; and fourth, ultra-deep sequencing of millions of barcode families from the single region-of-interest¹³. Bona fide mutations are differentiated from PCR and sequencing errors by virtue of being detected in all members of one barcode family¹³. The challenge of adapting MDS to the mammalian genome is maintaining the recovery of a sufficient number of analyzable barcode families (with at least 2 or 3 members) in a genome that is three orders of magnitude larger in size and weight^{14,15}. To this end, we optimized assay conditions (see Methods) for mammalian Kras (FIG. 6A) and barcode recovery (Table 4).

TABLE 4
Effect of sequencing depth on barcode recovery efficiency

| | | Raw reads/ | Barcodes | Barcodes | Barcodes |
|---|---|---|---|---|---|
| | Raw reads | number of | recovered | with | with |
| Sample | (×10⁶) | input cells | (×10⁶) | R > 1 (×10⁶) | R = 1 (%) |
| 1 | 2.1 | 0.5 | 1 | 0.5 | 47 |
| 2 | 3.2 | 0.8 | 0.6 | 0.4 | 23.5 |
| 3 | 4 | 1.0 | 1.1 | 0.7 | 32.4 |
| 4 | 4.6 | 1.2 | 1 | 0.6 | 33.2 |
| 5 | 4.9 | 1.3 | 1.3 | 0.9 | 28.9 |
| 6 | 6.4 | 1.6 | 1.1 | 0.9 | 25.3 |
| 7 | 8.4 | 2.2 | 1.4 | 1.1 | 22.7 |
| 8 | 8.7 | 2.2 | 1.1 | 0.9 | 19.4 |

R: number of independent reads sharing the same barcode

To validate the sensitivity of this mammalian version of MDS, we generated a panel of Kras-mutant plasmids, each comprised of Kras cDNA with a unique set of co-occurring double or triple mutations in the region encoded by exon 1 and/or exon 2 (Table 5). Each was spiked at specific concentrations into genomic DNA isolated from mouse embryonic fibroblasts (MEFs) or murine lungs to benchmark different levels of sensitivity. As the error rates of PCR and sequencing are unlikely to give the same two or three exact improper base calls, the actual frequency of mutants present in the sample was estimated by calculating the frequency of barcode families with the pre-engineered co-occurring mutations. The frequency of mutations determined by MDS was then compared against the aforementioned actual frequency. Using this approach, we demonstrated that MDS adapted for the transcribed strand of Kras exon 1 detected mutations at a sensitivity of 5×10⁻⁷ or 1 mutant per 2×10⁶ templates (FIG. 1A, FIG. 6B, and Table 5). We further validated the sensitivity of the MDS assay adapted for the non-transcribed strand of Kras exon 2 in the same fashion (FIG. 6C and Table 5). Thus, MDS optimized for mammalian genomic DNA detects mutations at a sensitivity potentially 20,000 times greater than conventional NGS.

TABLE 5
Frequency of spiked-in mutations
in MEF or murine lung genomic DNA

| Source of | | | | Frequency of | Frequency of |
|---|---|---|---|---|---|
| genomic | Target | Mutant | | mutant | mutant |
| DNA | exon | clone | Mutation | present | detected |
| MEF | Exon 1 | Clone 3 | A69T | 6.69E−07 | 5.13E−07 |
| | | | A74G | 6.69E−07 | 7.70E−07 |
| | | Clone 4 | A83T | 4.46E−07 | 2.57E−07 |
| | | | G73G | 4.46E−07 | 2.57E−07 |
| | | Clone 6 | G50A | 2.23E−06 | 3.08E−08 |
| | | | T56A | 2.23E−06 | 2.57E−06 |
| | | | A77G | 2.23E−06 | 3.34E−06 |
| | | Clone 10 | T62C | 3.79E−05 | 3.80E−05 |
| | | | A74T | 3.79E−05 | 3.80E−05 |
| | | Clone 12 | T6A | 0.000159 | 0.000164266 |
| | | | A61T | 0.000159 | 0.000166319 |
| | Exon 2 | Clone 1 | T155A | 8.82E−07 | 1.03E−06 |
| | | | T232C | 8.82E−07 | 2.06E−06 |
| | | Clone 5 | T141A | 0 | 0 |
| | | | G226T | 0 | 1.03E−06 |
| | | Clone 6 | T155C | 0 | 0 |
| | | | T234C | 0 | 1.03E−06 |
| | | Clone 8 | A182G | 8.82E−07 | 1.03E−06 |
| | | | T215C | 8.82E−07 | 1.03E−06 |
| | | | A219G | 8.82E−07 | 2.06E−06 |
| | | Clone 9 | C168A | 1.06E−05 | 9.26E−06 |
| | | | G229T | 1.06E−05 | 1.13E−05 |
| | | Clone 11 | A148G | 7.06E−06 | 7.20E−06 |
| | | | C235T | 7.06E−06 | 9.26E−06 |
| | | Clone 12 | A135G | 0.000103 | 9.98E−05 |
| | | | C150T | 0.000103 | 0.000117319 |
| | | | G247A | 0.000103 | 0.000117319 |
| Lung | Exon 1 | Clone 3 | A69T | 3.30E−07 | 3.76E−07 |
| | | | A74G | 3.30E−07 | 3.76E−07 |
| | | Clone 4 | A63T | 6.59E−07 | 1.13E−06 |
| | | | C73G | 6.59E−07 | 7.51E−07 |
| | | Clone 6 | G50A | 3.30E−06 | 4.13E−06 |
| | | | T56A | 3.30E−06 | 3.00E−06 |
| | | | A77G | 3.30E−06 | 2.63E−06 |
| | | Clone 10 | T62C | 4.95E−05 | 4.09E−05 |
| | | | A74T | 4.95E−05 | 4.09E−05 |
| | | Clone 12 | T6A | 0.000218 | 0.000199025 |
| | | | A61T | 0.000218 | 0.000197147 |

Capturing the initiating oncogenic mutation in Kras. Urethane induces pulmonary tumors driven by a Kras^{Q61/LR} oncogenic mutation^{1,3-5}, exemplifying the selectivity of this carcinogen at the level of tissue, isoform, position, and substitution. To elucidate the processes behind this RAS mutation tropism, we exposed A/J mice to urethane or the vehicle PBS via three daily intraperitoneal injections. After 1, 2, 3, and 4 weeks, genomic DNA was isolated from the lungs of four to seven mice from each condition. The non-transcribed strand of exon 2 of the endogenous Kras gene was then sequenced by MDS. To ensure abundant depth for mutant recovery and the accuracy of detected mutation frequency, samples with less than 1.5×10⁵ barcodes were excluded from analysis. For the remaining samples, mutation frequencies were summed by either nucleotide position or substitution type, normalized to control PBS, log₁₀ transformed, and then plotted as a heatmap (FIG. 1B,C).

This analysis identified the well-established^{1,3-5} oncogenic L (and to a lesser extent R) mutation at codon Q₆₁ preferentially in the urethane, but not PBS cohort of mice, as early as 1 week after exposure to this carcinogen. We confirm that these are initiating mutations, as they expanded over time indicative of tumor growth (FIG. 1B), although a longer time course would formally confirm a tumorigenic identity. We also independently confirm by droplet digital PCR¹⁶ the presence of the Q₆₁L mutation 4 week-post urethane exposure at a frequency similar to that determined by MDS (FIG. 6D). We thus capture and confirm the primordial initiating oncogenic mutation in Kras within days of exposure to urethane.

Substitution tropism. Previous whole-exome sequencing of urethane-induced tumors revealed a strong bias towards A>T/G substitutions³, consistent with ethenodeoxyadenosine adducts forming in vivo after urethane exposure^{17,18}. These substitutions were also detected in Kras by MDS at a high frequency, although A>T transversions were far more common than A>G transitions (FIG. 1C). In agreement with this bias, CA₁₈₂A→CTA gives rise to the dominant Q₆₁L oncogenic mutation in tumors of the A/J strain of mice exposed to urethane, while CA₁₈₂A→CGA gives rise to the rarer Q₆₁R oncogenic mutation^{1,5}. Still, the overall A/T content of murine genome¹⁵ is about 58%, and A>T/G substitutions represents two thirds of the possible base changes for this nucleotide. As such, this mutagenic signature is rather general compared to the extreme specificity of the initiating mutation. Further analysis of the mutation signature, using the log₁₀-transformed mutation frequencies of individual substitutions, revealed that the most prominent substitution detected by MDS in the lungs of mice after urethane (but not PBS) exposure at all time points was an A>T transversion within the context of a 5′ C and 3′ any nucleotide, namely a CAN trinucleotide (FIG. 1D, FIG. 7A,B). In agreement, a 5′ C was favored to some extent for A>T transversions in previous whole-exome sequencing of urethane-induced lung tumor³. The frequency of CAN→CTN mutations recovered in the urethane-exposed cohort remained constant over time in all but one case; CA₁₈₂A→CTA encoding the oncogenic Q₆₁L mutation expanded at subsequent time points ostensibly due to tumor growth (FIG. 1E). The same was true for the second most prominent urethane-specific substitution detected by MDS, an A>G transition preceded by 5′ C (FIG. 7B), where again CA₁₈₂A→CGA that gives rise the rarer Q₆₁R oncogenic mutation^{1,5} expanded over time (FIG. 7c). Substitutions other than CA₁₈₂A→CT/GA at codon 61 were rarely detected 1 week after urethane exposure (FIG. 1F), even though all the possible missense mutations at this codon generated by a single nucleotide substitution (Q₆₁L, R, K, E, P, and H) have been reported in human cancers in the COSMIC database¹⁹. As such, an A>T/G substitution preceded by C greatly increases the specificity of urethane mutations for codon 61, reducing the number of potential non-synonymous changes in both strands of the murine Kras gene by five-fold, from 616 to 120. The selectivity of these two substitutions after urethane exposure thus appears to be a major contributing factor to the substitution bias towards Q₆₁L/R mutations in Kras.

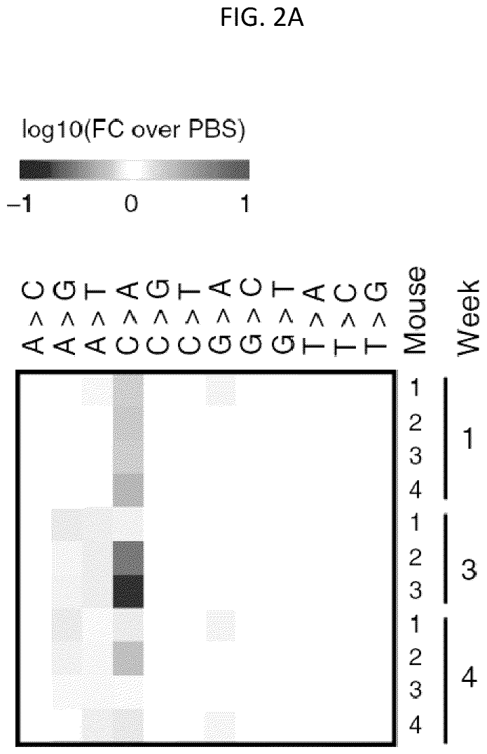

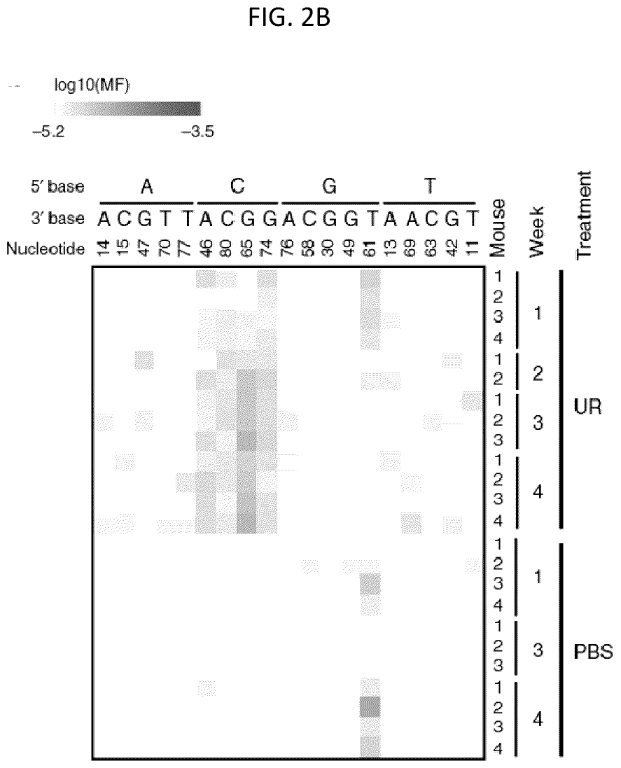

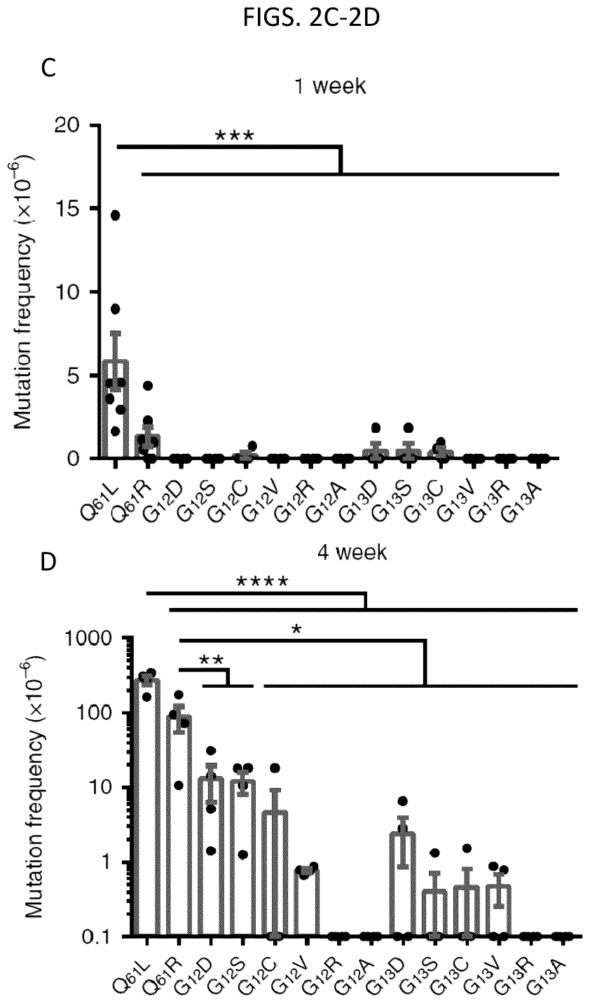

Position tropism. This bias of urethane for (C)A>T/G substitutions similarly argues against mutations arising at an appreciable level in codons 12 (G₃₄GT) or 13 (G₃₇GC) in exon 1, as neither fit the CAN pattern in either strand orientation. Related to this, despite the fact that oncogenic mutations at G₁₂, and to a lesser extend G₁₃, occur frequently in human cancers¹⁹ and when introduced into the lungs of mice are tumorigenic to varying degrees²⁰, they are rarely recovered from urethane-induced tumors³. We therefore sequenced the transcribed strand of exon 1 of Kras by MDS from genomic DNA isolated from the lungs of mice 1, 2, 3, and 4 weeks after exposure to urethane or PBS. To overcome interference from strand-specific background (see Methods), we also sequenced the non-transcribed strand of exon 1 of Kras by MDS from the lungs of mice at the 1- and 4-week time points. While the CAN→CTN signature was again preferentially detected 1 week after urethane exposure (FIG. 2A,B and FIG. 8A,B), indicating urethane mutagenesis occurred in this exon, oncogenic mutations were rarely recovered in either codon 12 or 13 (FIG. 2C). Interestingly, some G₁₂ and G₁₃ mutations were detected at a low frequency 4 weeks after urethane exposure (FIG. 2D). It is worth noting that oncogenic mutations at G₁₂ have been reported in urethane-induced tumors²¹, but are quite rare. This suggests that G₁₂ and G₁₃ mutations are induced by urethane exposure but remain below the limit of detection of MDS unless a certain degree of clonal expansion occurs. Similarly, while a Q₆₁H (A₁₈₃>T) mutation was rarely detected 1 week after urethane exposure (FIG. 1F), it was more prevalent in later samples (FIG. 1D). Collectively, these findings argue that the mutational position tropism of urethane can be ascribed in large part to a mutational bias of this environmental carcinogen towards CAN→CT/GN mutations.

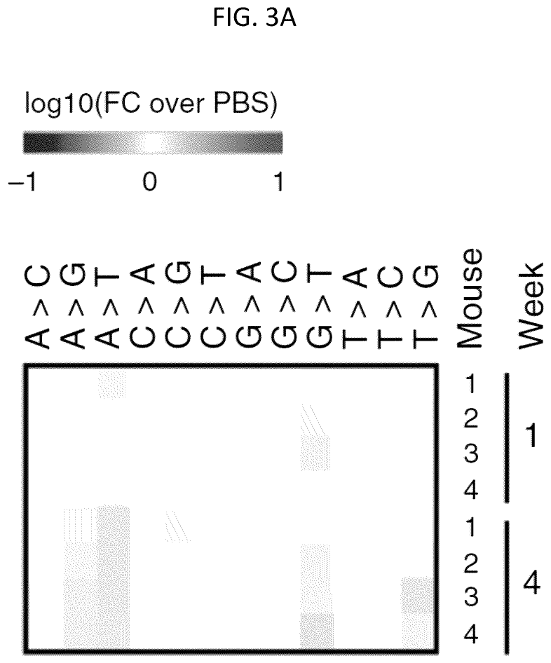

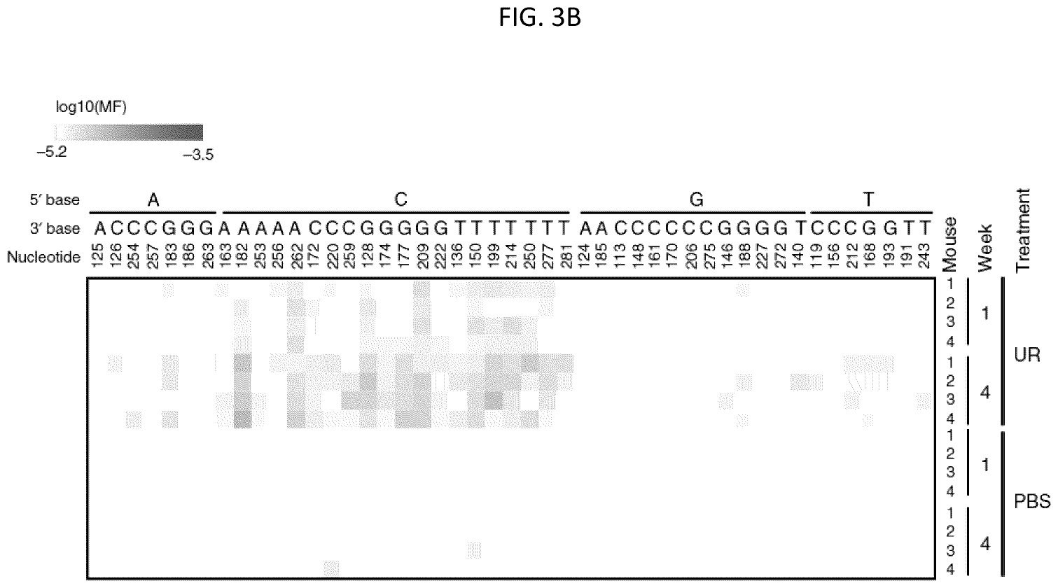

Isoform tropism. The other two Ras genes, Hras and Nras, encode the identical codon 61 (CAA). CAN→CT/GN substitutions at this codon generate the identical oncogenic Q61L/R mutations, which are well known to render Hras and Nras oncogenic^{22,23}. Despite this, oncogenic mutations in Hras or Nras are not recovered in urethane-induced lung tumors³. This suggests that either these loci are resistant in some manner to urethane mutagenesis or oncogenic mutations in these two genes are unable to initiate tumorigenesis. To differentiate between these two possibilities, we optimized the MDS assay to detect mutations in the non-transcribed strand of exon 2 in Hras (see Methods). We then applied this approach to genomic DNA isolated from the lungs of mice 1 and 4 weeks after exposure to urethane or PBS. We found a high prevalence of A>T followed by A>G mutations in exon 2 of Hras (FIG. 3A), with again CAN→CTN transversions being the predominant mutation in the urethane cohort, including the oncogenic CA₁₈₂A→CTA mutation in codon 61 (FIG. 3B). CAN→CTN transversions in Hras were detected somewhat less often than in Kras (FIG. 3C). However, while there was no difference in the frequency of all CAN→CTN transversions between Kras and Hras at 4 weeks, unlike in the case of Kras, oncogenic mutations in Hras did not expand appreciably over time (FIG. 3D). Hras therefore appears to acquire oncogenic mutations at a detectable frequency, but such mutations do not support tumorigenesis. This suggests that the isoform tropism of urethane is a product of the Hras locus and not an inability to induce oncogenic mutations at this site.

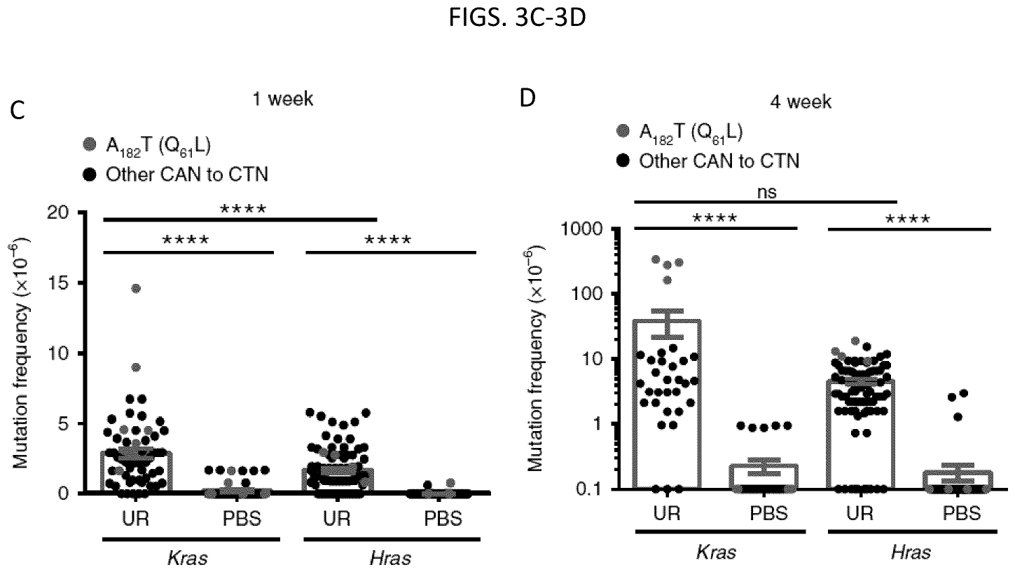

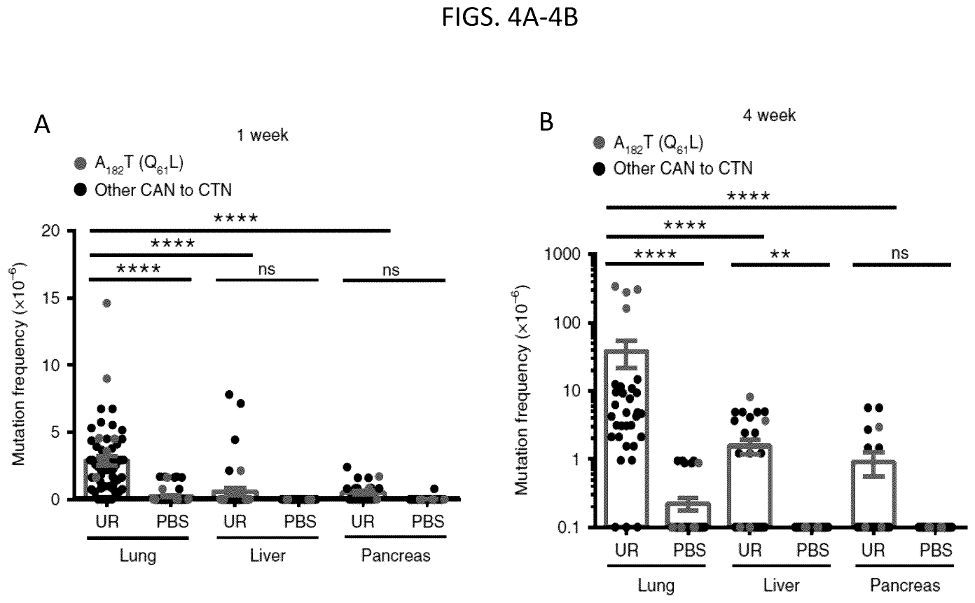

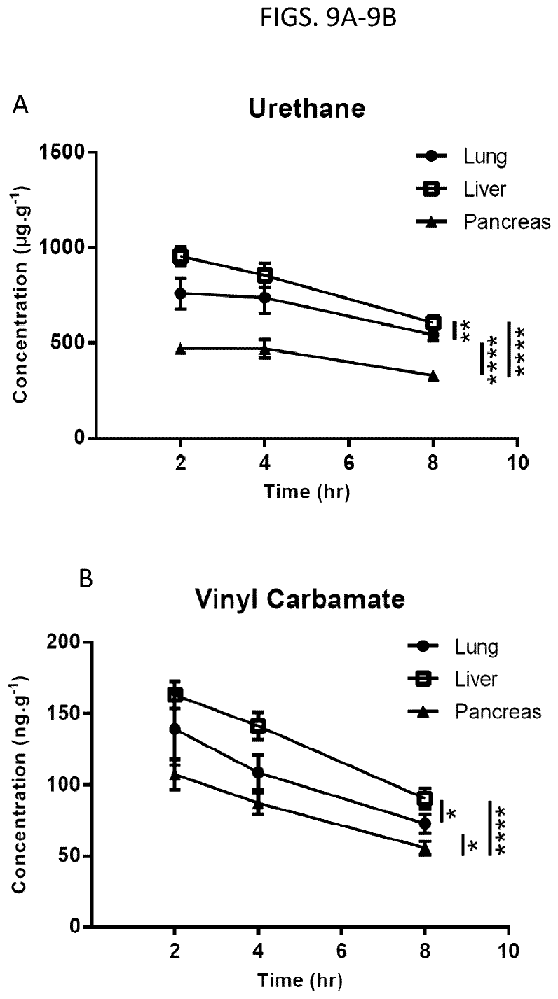

Organ tropism. Pulmonary lesions are the primary tumors arising in mice after intraperitoneal injections of urethane². However, activating an oncogenic Kras allele in a broad spectrum of murine organs has been documented to be tumorigenic²⁴. This begs the question of why urethane fails to induce other types of tumors. We thus analyzed the mutation status by MDS of the non-transcribed strand of exon 2 of Kras from lung compared to the liver and pancreas from mice 1 and 4 weeks after exposure to urethane versus PBS. The liver was chosen as in rare cases tumors develop in this organ during urethane carcinogenesis^{25,26}. The pancreas was chosen as it is sensitive to tumorigenesis by oncogenic Kras mutations^{27,28} but is not known to develop tumors after intraperitoneal injections of urethane². In comparison with the lung, significantly fewer CAN→CTN transversions were recovered in the liver and pancreas 1 and 4 weeks after urethane exposure (FIG. 4A,B). Again, unlike the situation in the lung, there was no overt expansion of Kras oncogenic mutations in the liver and pancreas over time, suggesting an absence of tumor growth (FIG. 4B). These findings argue that Kras acquires fewer mutations in these tissues after urethane exposure. To rule out the possibility that these tissues are less exposed to urethane, mice were injected with urethane or PBS and 2, 4, and 8 hours later the lungs, liver, and pancreas were removed and subjected to LC/MS/MS²⁹ to measure the levels of urethane and its active metabolite vinyl carbamate^{2,30}. Similar levels of both compounds were detected in the lungs and liver, but less in the pancreas over the three time points, with the terminal time point showing the highest concentration in the liver, followed by the lung, and then the pancreas (FIG. 9A,B), similar to results from the lung and liver using radiolabeled urethane³¹. These findings argue that the organ tropism of urethane appears to arise from differences in mutagenesis between tissues, rather than differential carcinogen exposure.

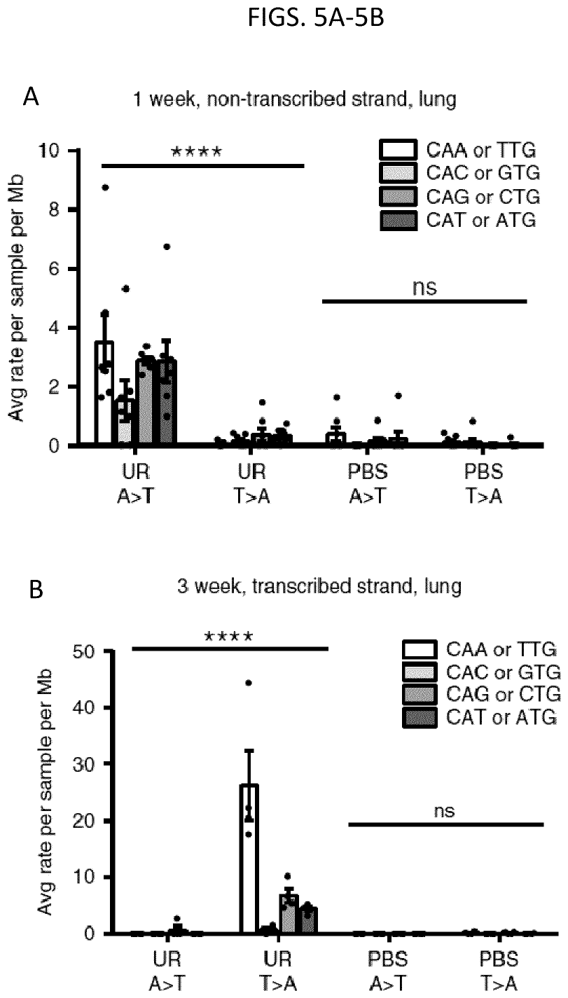

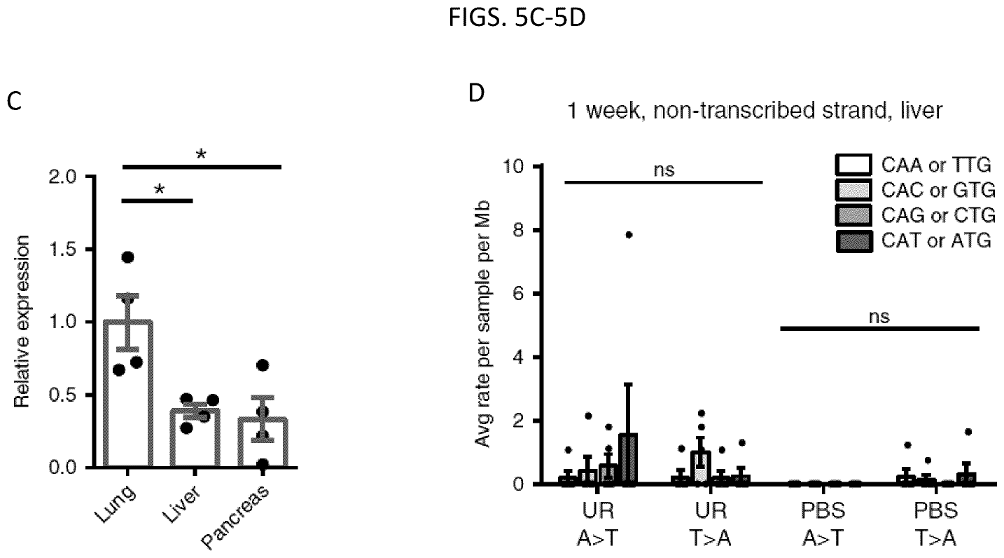

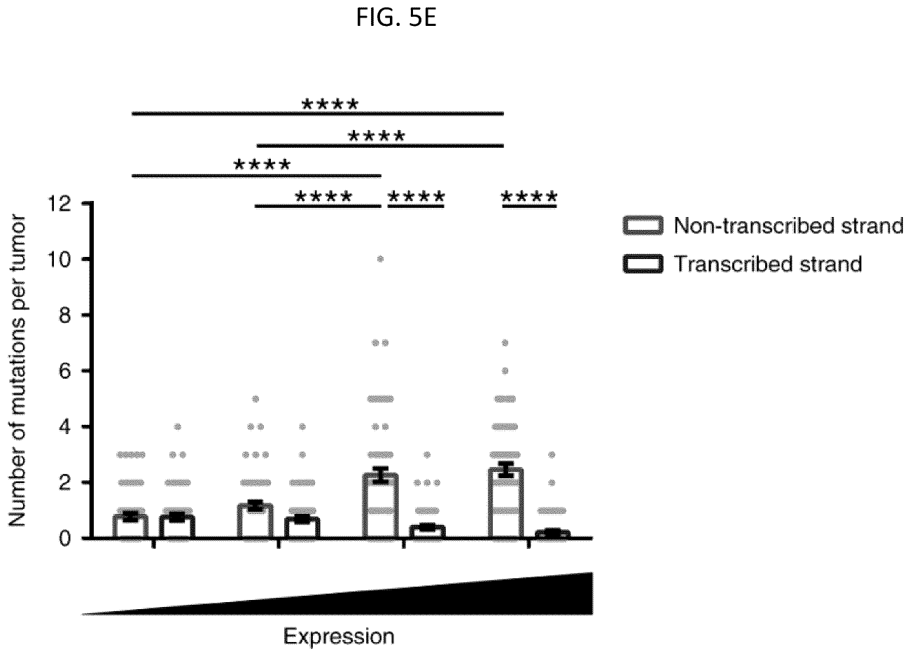

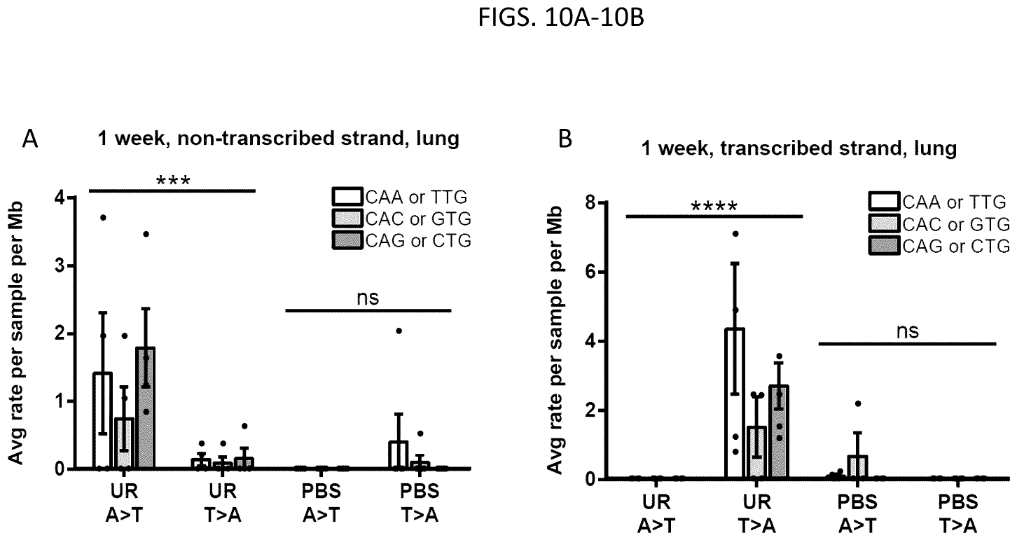

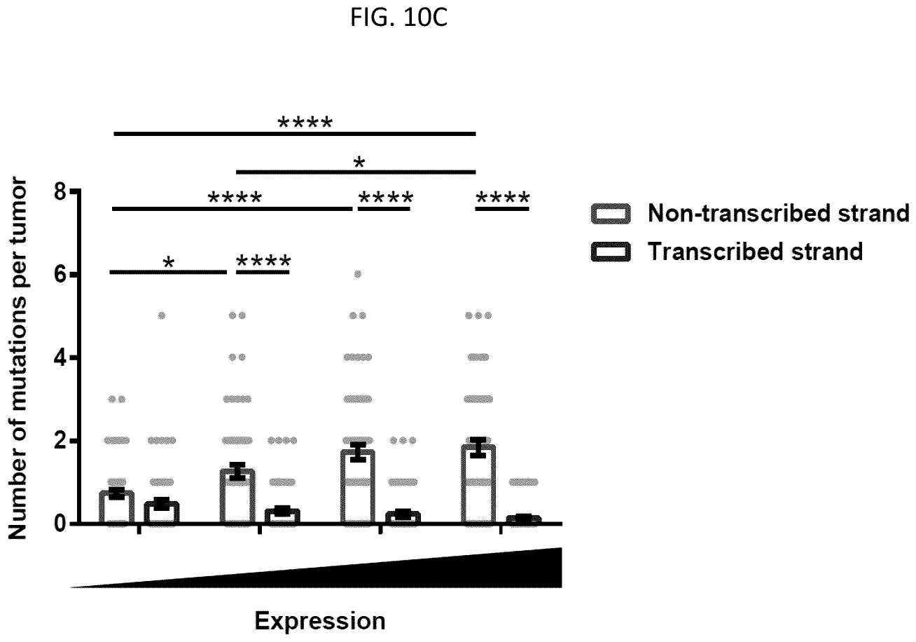

Strand bias. Given the above differences in the mutation frequency between different tissues, we revisited the MDS sequencing of the Kras locus, finding a bias towards mutations in the non-transcribed strand in mice exposed to urethane. In more detail, MDS targeting the non-transcribed strand of Kras exon 2 revealed that CAN→CTN, but not the complement NTG→NAG transversions, were the predominant mutations in the lungs of mice 1 week after exposure to urethane (FIG. 5A). To independently validate this result, we performed MDS targeting the opposite (transcribed) strand of exon 2 of Kras from the lungs of mice 3 weeks after exposure to urethane or PBS. This revealed a bias towards NTG→NAG over CAN→CTN transversions in the transcribed strand (FIG. 5B). The same was true for exon 1 of Kras, namely a bias towards CAN→CTN transversions in the non-transcribed strand compared to the transcribed strand, as determined from sequencing both strands by MDS (FIG. 10A,B). Thus, based on sequencing both strands in two different exons of Kras in the lung, urethane mutagenesis exhibits a bias for the non-transcribed strand.

Mutational strand asymmetry has been observed for other mutational processes^{32,33} and correlated with the transcriptional status of mutated genes³⁴. Kras mRNA levels determined by quantitative RT-PCR (RT-qPCR)³⁵ or RNA-seq³⁶ have been reported to be higher in the murine lung compared to the liver. In agreement, we validated the higher expression of Kras mRNA in lung compared to liver and pancreas by RT-qPCR (FIG. 5C). In addition, strand bias is not significant in the liver, consistent with a general lack of mutations detected in this organ after urethane exposure (FIG. 5D). Prompted by this, we examined the relationship between mutation frequency and gene expression using mutations detected in a previous published whole-exome sequencing of urethane-induced lung adenomas and adenocarcinomas³ and a published RNA-seq dataset generated from the adult mouse lung³. Genes were partitioned into quartiles based on expression level and the number of CAN→CTN transversions in non-transcribed or transcribed strand in each quartile determined. In agreement with the transcriptional strand bias revealed by MDS sequencing, CAN→CTN transversions increased with gene expression on the non-transcribed strand but decreased on the transcribed strand (FIG. 5E). The same trends were observed when the RNA-seq dataset for adult mouse lung from the mouse ENCODE project³⁷ was analyzed (FIG. 10C). Collectively, these findings point towards the organ tropism of urethane being related to the high transcription of Kras in the lung.

### Discussion

Here we adapted MDS, an error-corrected, high-throughput sequencing approach originally developed for use in microbiology¹³, to now detect extremely rare mutations in the mammalian genome at a sensitivity of up to 5×10⁻⁷ (1 mutant per 2×10⁶ templates). While we developed this assay to study RAS mutation tropism, MDS could find value in other applications, such as early detection³⁸. Nevertheless, by leveraging MDS to study the mutagenesis process at the earliest stage of tumorigenesis, we detected the initiating Q₆₁L/R mutations in Kras in the lungs of mice only days after exposure to urethane, capturing the very birth of cancer. We note that mutant allele-specific amplification^{39,40} and droplet digital PCR⁴¹ have documented Kras mutations after carcinogen exposure. However, we chose to develop MDS for the mammalian settings as these assays are either not as quantitative and sensitive^{39,42}, or are designed to examine pre-selected mutations^{41,43}. Indeed, capitalizing on the ability of MDS to detect any sequence variation in targeted regions of Ras genes at great sensitivity, we show at least three features underpinning the extreme mutational tropism of urethane- the mutational bias of this environmental carcinogen, transcription, and the gene locus.

With regards to the substitution and position bias of urethane, we demonstrate that the prevalence of Q₆₁L/R mutations arises in large part due to the known preference of urethane for A>T/G substitutions³, especially as we show here in the context of a 5′ C. This mutational bias, coupled with codon 61 containing a CAN trinucleotide that when the A is mutated to either T or G gives rise to an oncogenic L (CT₁₈₂A) or R (CG₁₈₂A) amino acid, favors the Kras^{Q61L/R} driver mutation characteristic of this carcinogen. Other oncogenic mutations at Q₆₁, G₁₂, or G₁₃ codons do not result from CA→CT/G substitutions, and in agreement, were rarely detected following urethane exposure. The implication being that a mutagenic preference may influence the type of initiating mutations in cancer. Similarly in humans, a CCT→CTC mutation characteristic of C>T transitions induced by UV encodes an activating P₂₉S mutation in RAC1 in sun-exposed melanoma⁴⁴.

While Q₆₁H, G₁₂, and G₁₃ oncogenic mutations in Kras, which are not favored by urethane mutagenesis, were rare or absent 1 week after urethane exposure, they were detectable 4 weeks later. This implies that extremely rare mutations induced by urethane, provided they have a favorable oncogenic outcome, may initiate tumorigenesis (although we cannot formally rule out that these were pre-existing mutations unveiled by a cooperating mutation induced by urethane). In agreement, while the Q₆₁L mutation is more frequent than Q₆₁R in urethane-induced lung tumors of the A/J mouse strain, the reverse is true in the B6 strain⁵. Similarly, the mutation spectrum of urethane is also shifted in a variety of mutant Ras backgrounds^{3,24,45,46}. If the mutagenesis preference of urethane is independent of strain background, the prevalence of the Q₆₁R mutation suggest that this less common mutation is more conducive to tumor initiation in the B6 strain. As such, the most dominant mutation of a mutagen may not always dictate the initiating event, echoing the common discordance between the mutagenic signatures and the putative initiating mutation in certain human cancers⁴⁷⁻⁴⁹.

Another fascinating feature of urethane mutagenesis revealed by MDS sequencing relates to isoform tropism. We found that codon 61 was readily mutated in Hras in lung tissue, yet the oncogenic Hras allele was not expanded appreciably over time. This suggests that either Hras^{Q61L} is not as oncogenic as Kras^{Q61L} or the encoded protein is expressed too low (or high) to be tumorigenic. In support of the first, RAS isoforms differ in their residency at different membranes⁵⁰ and the composition of proteins within the immediate vicinity differs between RAS isoforms^{51,52}, with proteins like PIP5K1A⁵², calmodulin⁵³, galectin-3⁵⁴, and so forth documented to specifically associate with KRAS. In support of the second, a Kras allele whereby the 3′ end was replaced with Hras exons to encode Hras protein was found mutated in urethane-induced tumors⁵⁵, indicating that under a Kras promoter Hras^{Q61L} is indeed oncogenic in the lung. Whether the inability of oncogenic mutations in Hras to promote lung tumorigenesis is because the protein is less oncogenic, expressed too low, too high, combinations thereof, or for other reasons^{7,24,56,57} remains to be elucidated. Nevertheless, the finding that Hras is mutated yet such mutations are not recovered in lung tumors³ after urethane exposure is in itself an important finding, and perhaps related, of the three RAS genes, HRAS is mutated the least often in human cancers^{6,7,58}.

With regards to organ tropism, a very different mechanism appears to be at play. In this case, we found that Kras is rarely mutated in the liver and pancreas, despite the presence of the carcinogen. While a number of factors could contribute to this variation in mutagenesis⁵⁹⁻⁶¹, one notable difference is that Kras mRNA levels are higher in the lung compared to these other tissues, suggestive of increased transcription. In fact, the lung was found to have the second highest levels of Kras mRNA of 15 adult murine tissues analyzed, second only to the brain³⁵. Kras expression in the mouse lung also correlates with strain susceptibility to urethane carcinogenesis^{62,63}. Related, we discovered that the non-transcribed strand of Kras is preferentially mutated, which for other mutagens has been linked to transcription-coupled repair of the transcribed strand⁶⁴ or transcription-coupled damage of the displaced, non-transcribed strand³⁴. Indeed, we found a global correlation between mRNA levels and the mutation frequency of urethane. This is not to say that there is a universal concordance between high gene transcription and an elevated mutation frequency of the non-transcribed strand. Indeed, high transcription has been associated with a lower mutation frequency in chromatin-dense genomic regions in cutaneous squamous cell carcinomas⁶⁵. Thus, the type of cancer, mutational process, specific genes, and so forth may influence the bias of a mutagenic process. In the case of urethane however, we suggest that the tissue tropism is related to the high transcription of Kras in the lung, increasing the susceptibility of this gene to urethane mutagenesis.

In humans, there are also very distinct patterns to RAS mutations at the level of the organ (e.g., RAS is commonly mutated in pancreatic but rarely in breast cancer), isoform (e.g. KRAS is mutated in lung cancer while NRAS is mutated in melanoma), position (e.g. G₁₂ is mutated in CMML while Q₆₁ is mutated in thyroid carcinoma), and substitution (e.g. G₁₂V is the primary mutation in bladder carcinoma while it is G₁₂S in mouth carcinoma). There is no definitive mechanism to explain this phenomenon, although the pattern itself has been widely reported for decades^{6,7,24,58,66-68}. In this regard, the extreme specificity of urethane carcinogenesis for Kras^{Q61L/R}-mutant pulmonary tumors may inform the basic principles of the RAS mutation patterns observed in these clinical samples. Admittedly, urethane is not a major environmental carcinogen in humans compared to, for example, tobacco smoke. Kras^{Q61L/R} mutations are also rare in human lung cancers'. With these two provisos, we speculate that the RAS mutation tropism of human cancers may similarly be a product of mutagenesis selectivity factors, for example the specificity of the mutagenic process or susceptibility of a specific locus to mutations, and selection factors, for example differences in the oncogenic activity of one isoform over another. Moreover, it is entirely possible, if not likely, that different combinations of these or even other factors such as cooperating mutations, as elegantly demonstrated in MNU carcinogenesis³, cell type⁶⁹, signaling intensity⁴⁵, and so forth²⁴ underlie the RAS mutation tropism human cancers. As such, each cancer initiating event may be molded by a unique set of factors, each with varying influence.

### REFERENCES

1. You, M., Candrian, U., Maronpot, R.R., Stoner, G.D. & Anderson, M.W. Activation of the Ki-ras protooncogene in spontaneously occurring and chemically induced lung tumors of the strain A mouse. Proc Natl Acad Sci USA

86, 3070-4 (1989).
2. Forkert, P.G. Mechanisms of lung tumorigenesis by ethyl carbamate and vinyl carbamate. Drug Metab Rev 42, 355-78 (2010).
3. Westcott, P.M. et al. The mutational landscapes of genetic and chemical models of Kras-driven lung cancer. Nature 517, 489-92 (2015).
4. Nuzum, E.O., Malkinson, A.M. & Beer, D.G. Specific Ki-ras codon 61 mutations may determine the development of urethan-induced mouse lung adenomas or adenocarcinomas. Mol Carcinog 3, 287-95 (1990).
5. Dwyer-Nield, L.D. et al. Epistatic interactions govern chemically-induced lung tumor susceptibility and Kras mutation site in murine C57BL/6J-ChrA/J chromosome substitution strains. Int J Cancer 126, 125-32 (2010).
6. Cox, A.D., Fesik, S.W., Kimmelman, A.C., Luo, J. & Der, C.J. Drugging the undruggable RAS: Mission possible? Nat Rev Drug Discov 13, 828-51 (2014).
7. Prior, I.A., Lewis, P.D. & Mottos, C. A comprehensive survey of Ras mutations in cancer. Cancer Res 72, 2457-67 (2012).
8. Hernandez, L.G. & Forkert, P.G. In vivo mutagenicity of vinyl carbamate and ethyl carbamate in lung and small intestine of F1 (Big Blue x A/J) transgenic mice. Int J Cancer 120, 1426-33 (2007).
9. Fox, E.J., Reid-Bayliss, K.S., Emond, M.J. & Loeb, L.A. Accuracy of next generation sequencing platforms. Next Gener Seq Appl 1, 1000106 (2014).
10. Kinde, I., Wu, J., Papadopoulos, N., Kinzler, K.W. & Vogelstein, B. Detection and quantification of rare mutations with massively parallel sequencing. Proc Natl Acad Sci USA 108, 9530-5 (2011).
11. Schmitt, M.W. et al. Detection of ultra-rare mutations by next-generation sequencing. Proc Natl Acad Sci USA 109, 14508-13 (2012).
12. Lou, D.I. et al. High-throughput DNA sequencing errors are reduced by orders of magnitude using circle sequencing. Proc Natl Acad Sci USA 110, 19872-7 (2013).
13. Jee, J. et al. Rates and mechanisms of bacterial mutagenesis from maximum-depth sequencing. Nature 534, 693-6 (2016).
14. Blattner, F.R. et al. The complete genome sequence of Escherichia coli K-12. Science 277, 1453-62 (1997).
15. Mouse Genome Sequencing Corsortium. et al. Initial sequencing and comparative analysis of the mouse genome. Nature 420, 520-62 (2002).

16. Hindson, B.J. et al. High-throughput droplet digital PCR system for absolute quantitation of DNA copy number. Anal Chem 83, 8604-10 (2011).
17. Fernando, R.C., Nair, J., Barbin, A., Miller, J.A. & Bartsch, H. Detection of 1N⁶-ethenodeoxyadenosine and 3,N⁴-ethenodeoxycytidine by immunoaffinity/³² P-postlabelling in liver and lung DNA of mice treated with ethyl carbamate (urethane) or its metabolites. Carcinogenesis 17, 1711-8 (1996).
18. Forkert, P.G. et al. Oxidation of vinyl carbamate and formation of 1,1V 6 -ethenodeoxyadenosine in murine lung. Drug Metab Dispos 35, 713-20 (2007).
19. Tate, J.G. et al. COSMIC: the Catalogue Of Somatic Mutations In Cancer. Nucleic Acids Res 47, D941-7 (2019).
20. Winters, I.P. et al. Multiplexed in vivo homology-directed repair and tumor barcoding enables parallel quantification of Kras variant oncogenicity. Nat Commun 8, 2053 (2017).
21. Borrego, A. et al. Germline control of somatic Kras mutations in mouse lung tumors. Mol Carcinog 57, 745-51 (2018).
22. Burd, C.E. et al. Mutation-specific RAS oncogenicity explains NRAS codon 61 selection in melanoma. Cancer Discov 4, 1418-29 (2014).
23. Kiessling, M.K. et al. Mutant HRAS as novel target for MEK and mTOR inhibitors. Oncotarget 6, 42183-96 (2015).
24. Li, S., Balmain, A. & Counter, C.M. A model for RAS mutation patterns in cancers: finding the sweet spot. Nat Rev Cancer 18, 767-77 (2018).
25. Dragani, T.A., Manenti, G. & Della Porta, G. Quantitative analysis of genetic susceptibility to liver and lung carcinogenesis in mice. Cancer Res 51, 6299-303 (1991).
26. Heston, W.E., Vlahakis, G. & Deringer, M.K. High incidence of spontaneous hepatomas and the increase of this incidence with urethan in C3H, C3Hf, and C3He male mice. J Natl Cancer Inst 24, 425-35 (1960).
27. Guerra, C. et al. Chronic pancreatitis is essential for induction of pancreatic ductal adenocarcinoma by K-Ras oncogenes in adult mice. Cancer Cell 11, 291-302 (2007).
28. Hingorani, S.R. et al. Preinvasive and invasive ductal pancreatic cancer and its early detection in the mouse. Cancer Cell 4, 437-50 (2003).
29. Grebe, S.K. & Singh, R.J. LC-MS/MS in the clinical laboratory—Where to from here? Clin Biochem Rev 32, 5-31 (2011).
30. Guengerich, F.P. & Kim, D.H. Enzymatic oxidation of ethyl carbamate to vinyl carbamate and its role as an intermediate in the formation of 1,N⁶-ethenoadenosine. Chem Res Toxicol 4, 413-21 (1991).
31. Nomeir, A.A., Ioannou, Y.M., Sanders, J.M. & Matthews, H.B. Comparative metabolism and disposition of ethyl carbamate (urethane) in male Fischer 344 rats and male B6C3F1 mice. Toxicol Appl Pharmacol 97, 203-15 (1989).
32. Alexandrov, L.B. et al. Signatures of mutational processes in human cancer. Nature 500, 415-21 (2013).
33. Kucab, J.E. et al. A compendium of mutational signatures of environmental agents. Cell 177, 821-36 (2019).
34. Haradhvala, N.J. et al. Mutational strand asymmetries in cancer genomes reveal mechanisms of DNA damage and repair. Cell 164, 538-49 (2016).
35. Newlaczyl, A.U., Coulson, J.M. & Prior, I.A. Quantification of spatiotemporal patterns of Ras isoform expression during development. Sci Rep 7, 41297 (2017).
36. Li, B. et al. A comprehensive mouse transcriptomic bodymap across 17 tissues by RNA-seq. Sci Rep 7, 4200 (2017).
37. Shen, Y. et al. A map of the cis-regulatory sequences in the mouse genome.Nature 488, 116-20 (2012).
38. Phallen, J. et al. Direct detection of early-stage cancers using circulating tumor DNA. Sci Transl Med 9, eaan2415 (2017).

39. Ichikawa, T. et al. The activation of K-ras gene at an early stage of lung tumorigenesis in mice. Cancer Lett 107, 165-70 (1996).
40. Yano, T., Yuasa, M., Murakami, A., Ichikawa, T. & Hagiwara, K. The detection of chemically initiated cells having the mutation of K-ras gene at an early stage of lung carcinogenesis in mice. Anal Biochem 244, 187-9 (1997).
41. Spella, M. et al. Club cells form lung adenocarcinomas and maintain the alveoli of adult mice. Elife 8, e45571 (2019).
42. van Mansfeld, A.D. & Bos, J.L. PCR-based approaches for detection of mutated ras genes. PCR Methods Appl 1, 211-6 (1992).
43. Pender, A. et al. Efficient genotyping of KRAS mutant non-small cell lung cancer using a multiplexed droplet digital PCR approach. PLoS One 10, e0139074 (2015).
44. Krauthammer, M. et al. Exome sequencing identifies recurrent somatic RAC1 mutations in melanoma. Nat Genet 44, 1006-14 (2012).
45. Pershing, N.L. et al. Rare codons capacitate Kras-driven de novo tumorigenesis. J Clin Invest 125, 222-33 (2015).
46. Huang, L., Carney, J., Cardona, D.M. & Counter, C.M. Decreased tumorigenesis in mice with a Kras point mutation at C118. Nat Commun 5, 5410 (2014).
47. Temko, D., Tomlinson, I.P.M., Severini, S., Schuster-Bockler, B. & Graham, T.A. The effects of mutational processes and selection on driver mutations across cancer types. Nat Commun 9, 1857 (2018).
48. Buisson, R. et al. Passenger hotspot mutations in cancer driven by APOBEC3A and mesoscale genomic features. Science 364, eaaw2872 (2019).
49. Dietlein, F. et al. Identification of cancer driver genes based on nucleotide context. Nat Genet 52, 208-18 (2020).
50. Hancock, J.F. Ras proteins: different signals from different locations. Nat Rev Mol Cell Biol 4, 373-84 (2003).
51. Kovalski, J.R. et al. The functional proximal proteome of oncogenic Ras includes mTORC2. Mol Cell 73, 830-44 e12 (2019).
52. Adhikari, H. & Counter, C.M. Interrogating the protein interactomes of RAS isoforms identifies PIP5K1A as a KRAS-specific vulnerability. Nat Commun 9, 3646 (2018).
53. Villalonga, P. et al. Calmodulin binds to K-Ras, but not to H- or N-Ras, and modulates its downstream signaling. Mol Cell Biol 21, 7345-54 (2001).
54. Elad-Sfadia, G., Haklai, R., Balan, E. & Kloog, Y. Galectin-3 augments K-Ras activation and triggers a Ras signal that attenuates ERK but not phosphoinositide 3-kinase activity. J Biol Chem 279, 34922-30 (2004).
55. To, M.D. et al. Kras regulatory elements and exon 4A determine mutation specificity in lung cancer. Nat Genet 40, 1240-4 (2008).
56. McCreery, M.Q. & Balmain, A. Chemical carcinogenesis models of cancer: back to the future. Annu Rev Cancer Biol 1, 295-312 (2017).
57. Simanshu, D.K., Nissley, D.V. & McCormick, F. RAS proteins and their regulators in human disease. Cell 170, 17-33 (2017).
58. Stephen, A.G., Esposito, D., Bagni, R.K. & McCormick, F. Dragging ras back in the ring. Cancer Cell 25, 272-81 (2014).
59. Adar, S., Hu, J., Lieb, J.D. & Sancar, A. Genome-wide kinetics of DNA excision repair in relation to chromatin state and mutagenesis. Proc Natl Acad Sci USA 113, E2124-33 (2016).
60. Hoffler, U. & Ghanayem, B.I. Increased bioaccumulation of urethane in CYP2E1−/− versus CYP2E1+/+ mice. Drug Metab Dispos 33, 1144-50 (2005).
61. Supek, F. & Lehner, B. Clustered mutation signatures reveal that error-prone DNA repair targets mutations to active genes. Cell 170, 534-47 (2017).
62. To, M.D. et al. A functional switch from lung cancer resistance to susceptibility at the Pasl locus in Kras2LA2 mice. Nat Genet 38, 926-30 (2006).
63. Dassano, A. et al. Mouse pulmonary adenoma susceptibility 1 locus is an expression QTL modulating Kras-4A. PLoS Genet 10, e1004307 (2014).
64. Hanawalt, P.C. & Spivak, G. Transcription-coupled DNA repair: two decades of progress and surprises. Nat Rev Mol Cell Biol 9, 958-70 (2008).
65. Zheng, C.L. et al. Transcription restores DNA repair to heterochromatin, determining regional mutation rates in cancer genomes. Cell Rep 9, 1228-34 (2014).
66. Pylayeva-Gupta, Y., Grabocka, E. & Bar-Sagi, D. RAS oncogenes: weaving a tumorigenic web. Nat Rev Cancer 11, 761-74 (2011).
67. Haigis, K.M. KRAS alleles: the devil is in the detail. Trends Cancer 3, 686-97 (2017).
68. Bos, J.L. ras oncogenes in human cancer: a review. Cancer Res 49, 4682-9 (1989).
69. Xu, X. et al. Evidence for type II cells as cells of origin of K-Ras-induced distal lung adenocarcinoma. Proc Natl Acad Sci USA 109, 4910-5 (2012).
70. Morgenstern, J.P. & Land, H. Advanced mammalian gene transfer: high titre retroviral vectors with multiple drug selection markers and a complementary helper-free packaging cell line. Nucleic Acids Res 18, 3587-96 (1990).
71. O'Hayer, K.M. & Counter, C.M. A genetically defined normal human somatic cell system to study ras oncogenesis in vivo and in vitro. Methods Enzymol 407, 637-47 (2006).
72. Norrander, J., Kempe, T. & Messing, J. Construction of improved M13 vectors using oligodeoxynucleotide-directed mutagenesis. Gene 26, 101-6 (1983).
73. Afgan, E. et al. The Galaxy platform for accessible, reproducible and collaborative biomedical analyses: 2018 update. Nucleic Acids Res 46, W537-44 (2018).
74. Zhang, J., Kobert, K., Flouri, T. & Stamatakis, A. PEAR: a fast and accurate Illumina Paired-End reAd mergeR. Bioinformatics 30, 614-20 (2014).
75. Arbeithuber, B., Makova, K.D. & Tiemann-Boege, I. Artifactual mutations resulting from DNA lesions limit detection levels in ultrasensitive sequencing applications. DNA Res 23, 547-59 (2016).
76. Rowlands, V. et al. Optimisation of robust singleplex and multiplex droplet digital PCR assays for high confidence mutation detection in circulating tumour DNA. Sci Rep 9, 12620 (2019).
77. Livak, K.J. & Schmittgen, T.D. Analysis of relative gene expression data using real-time quantitative PCR and the 2(-Delta Delta C(T)) Method. Methods 25, 402-8 (2001).

### Example 2: High-Throughput Detection of Mutations in Human Ras Genes

In the following example, the inventors adapted an error-corrected, high-throughput sequencing approach to detect mutations in human Ras genes with high sensitivity. Similar methods as described in Example 1 were modified to be used for human gene detection as described herein.

### Materials and Methods

Isolation of genomic DNA. Cells were resuspended in lysis buffer (100 mM NaCl, 10 mM Tris pH 7.6, 25 mM EDTA pH 8.0, and 0.5% SDS in H₂O, supplemented with 20 μg·ml⁻¹ RNase A (Sigma)). Samples were incubated at 37° C. for 1 hour. 2 μlof 800 U·ml⁻¹ proteinase K (NEB) was then added to each sample, the samples were vortexed, and then incubated at 55° C. overnight. Genomic DNA was isolated by phenol/chloroform extraction followed by ethanol precipitation using standard procedures and quantified using Qubit fluorometer.

Maximum depth sequencing (MDS). The MDS assay' was adapted for mammalian Ras genes as follows. 20-50 μg of genomic DNA was incubated with Stul (NEB) for analysis of the transcribed strand of Kras exon 1, Hinfl for analysis of the non-transcribed strand of Kras exon 1, or Xmnl (NEB) for analysis of the non-transcribed strand of Kras exon 2. Reaction conditions were 5 units of the indicated restriction enzyme and per 1 μg DNA per 20 μl reaction (e.g., 20 μg genomic DNA, 5 μl enzyme (20 units/μl), and 40 μl 10×buffer in 400 μl reaction). Digested genomic DNA was column purified using QIAquick PCR Purification Kit following the manufacturer's protocol (Qiagen) and resuspended in ddH₂O (35 μl H₂O per 10 μg DNA). The barcode and adaptor were added to the target DNA by incubating purified DNA with the appropriate adaptor-barcode primer (see below) for one cycle of PCR. PCR reactions were comprised of 10 μg DNA, 2.5 μl of 10 μM adaptor-barcode primer, 4 μl of 2.5 mM dNTP, 10 μl of 5×buffer (NEB), and 0.5 μl Q5® Hot Start High-Fidelity DNA Polymerase (NEB) in a total volume of 50 μl. The number of PCR reactions was scaled according to the amount of DNA. PCR conditions were 98° C. for 1 minute, adaptor-barcode primer annealing temperate (see below) for 15 seconds, and 72° C. for 1 minute. 1 μl of 20,000 U·ml⁻¹ exonuclease I (NEB) and 5 μl of 10×exonuclease I buffer (NEB) was then added to each 50 μl reaction to remove unused adaptor-barcode primers and incubated at 37° C. for 1 hour and then 80° C. for 20 minutes. Processed DNA were column purified using QIAquick PCR Purification Kit as above and resuspended in ddH2O (35 μl H₂O per column). The concentration of purified product was measured with SimpliNano spectrophotometer (GE Healthcare Life Sciences). Samples were linear amplified with forward adaptor primer (see below). PCR reactions were comprised of 1.5 μg DNA, 2.5 μl of 10 μM forward adaptor primer, 4 μl of 2.5 mM dNTP, 10 μl of 5×buffer (NEB), and 0.5 μl Q5® Hot Start High-Fidelity DNA Polymerase (NEB) in a total volume of 50 μl. The number of PCR reactions was scaled according to the amount of DNA. PCR conditions were as follows: 12 cycles of 98° C. for 15 seconds, 70° C. for 15 seconds, 72° C. for 8 seconds. 2.5 μl of 10 μM exon-specific reverse primer (see below) and 2.5 μl of 10 μM reverse adaptor primer (see below) were then added to each 50 μl reaction. The mixtures were then subjected to 20 cycles of exponential amplification. PCR conditions were as follows: 4 cycles of 98° C. for 15 seconds, exon-specific reverse primer annealing temperature (see below) for 15 seconds, 72° C. for 8 seconds, 16 cycles of 98° C. for 15 seconds, 70° C. for 15 seconds, and 72° C. for 8 seconds. The final library was size selected and purified with Ampure XP beads according to the manufacturer's protocol (Beckman Coulter). Sequencing was performed using HiSeq 2500 100 bp PE rapid run, HiSeq 4000 150 bp PE or NovaSeq 6000 S Prime 150 bp PE at Duke Center for Genomic and Computational Biology. For the optimization of barcode recovery, the same amount of genomic DNA was processed in parallel by MDS assay targeting Kras exon 1 transcribed strand and the PCR products were pooled together at different concentrations in one library to obtain different sequencing depths.

### Primers for Maximum Depth Sequencing

Adaptor-barcode primer: [5′-forward adaptor
sequence][barcode sequence][ROI-specific
sequence-3′] [Forward adaptor sequence] =
(SEQ ID NO: 1)
5′-TACGGCGACCACCGAGATCTACACTCTTTCCCTACACGACGCTCTTC
CGATCT-3′
(SEQ ID NO: 5)
[Barcode] = NNNNNNNNNNNNNN
[ROI-specific sequence] = Table 6

TABLE 6
[ROI-specific sequence] of adaptor-barcode primer

| | SEQ | | Annealing |
|---|---|---|---|
| ROI-specific | ID | | Temp. |
| sequence | NO: | Sequence (5′→3′) | (°C) |
| Kras exon 1 StuI | 6 | CCTGCTGAAAATGACT | 60 |
| (transcribed strand) | | GAA | |
| Kras exon 1 HinfI | 9 | CTGAATTAGCTGTATC | 60 |
| (non-transcribed | | GTCAAG | |
| strand) | | | |
| Kras exon 2 XmnI | 11 | TCTTCAAATGATTTAG | 59 |
| (non-transcribed | | TATTATTTATGGC | |
| strand) | | | |

Forward-adaptor primer:
(SEQ ID NO: 2)
5′-AATGATACGGCGACCACCGAGAT-3′
(annealing temperature: 70° C.)
Exon-specific reverse primer:
[5′-reverse adaptor sequence]
[ROI-specific sequence-3′]
[Reverse adaptor sequence] =
(SEQ ID NO: 3)
5′-CAAGCAGAAGACGGCATACGAGATGTGACTGGAGTTCAGACGTGTGC
TCTTCCGATCT-3′

| | SEQ | | Annealing |
|---|---|---|---|
| ROI-specific | ID | | Temp. |
| sequence | NO: | Sequence (5′→3′) | (° C.) |
| Kras exon 1 StuI | 7 | CTCTATTGTTGGATCA | 59 |
| (transcribed | | TATTCGT | |
| strand) | | | |
| Kras exon 1 StuI | 8 | TAGCTGTATCGTCAAG | 61 |
| (transcribed | | GC | |
| strand) | | | |
| Kras exon 1 HinfI | 10 | ATGACTGAATATAAAC | 61 |
| (non-transcribed | | TTGTGGTAGT | |
| strand) | | | |
| Kras exon 2 XmnI | 12 | GATTCCTACAGGAAGC | 61 |
| | | AAGT | |

| | Reverse-adaptor primer = |
|---|---|
| (SEQ ID NO: 4) | |
| | 5′-CAAGCAGAAGACGGCATACGAGA-3′ |
| | (annealing temperature: 70° C.) |

All primers were synthesized by Integrated DNA Technologies (IDT).

For the experiment optimizing barcode recovery, the reads were trimmed down to the barcode and grouped into families by barcode. The number of families containing 1 read and ≥2 reads was then counted, respectively.

### Results

Adapting MDS to the mammalian genome. Similar to endeavors discussed in Example 1, we optimized assay conditions (see Methods) for the detection of mutations with the human KRAS gene (FIG. 11).

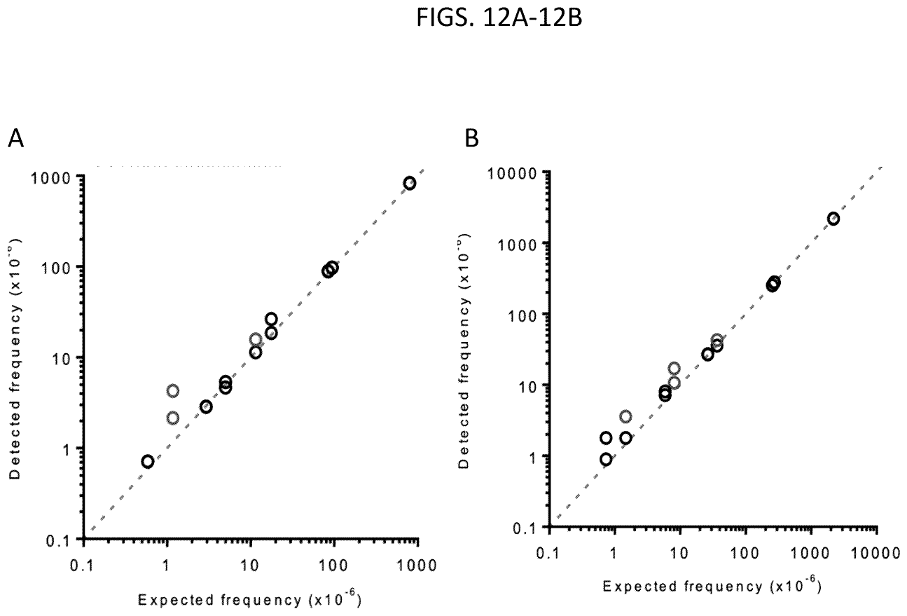

Adapting K-MDS to detect G_{12/13} mutations in human KRAS. We modified the K-MDS assay for the transcribed strand of exon 1 of human KRAS. In brief, human 293 T gDNA was spiked with a panel of KRAS^{GAT}* DNA templates which all contained a G₃₄GT→GAT mutation encoding the common G₁₂D oncogenic mutation and a second unique co-occurring mutation (*) to benchmark specific concentrations of template, ranging from 1×10⁻³ to 10⁻⁶. Using this panel we tested various restriction enzymes, primers, annealing temperatures, linear and exponential amplification cycles, and so forth to optimize K-MDS for human KRAS exon 1. As above, the actual frequency of G₁₂D mutants present in the sample was estimated by calculating the frequency of barcode families with the pre-engineered co-occurring mutations. We find complete concordance between detecting a single mutation alone versus with a co-occurring mutation down to the lowest sensitivity assayed (1×10⁻⁶, FIG. 12A). We note that C/G>T substitutions can arise by deamination of cytosine or methyl-cytosine and oxidation of guanine during library preparation^{19,20} or from mis-incorporation not yet repaired⁶. However, we show that only C>T and G>T mutants engineered in the KRAS template were identified (FIG. 12A). Nevertheless, to reduce C>T or G>T false-positives we also optimized K-MDS for the non-transcribed strand of human KRAS exon 1, as bona fide C>T or G>T mutations will cause complementary G>A or C>A mutations in this strand. Again, we find perfect concordance between the actual and predicted mutations in this strand (FIG. 12B). The same results were observed in three biological replicate experiments. Thus, K-MDS detects mutations in human a KRAS exon 1 template at a sensitivity of at least 1×10⁻⁶.

### References for Example 2

1. You, M., Candrian, U., Maronpot, R.R., Stoner, G.D. & Anderson, M.W. Activation of the Ki-ras protooncogene in spontaneously occurring and chemically induced lung tumors of the strain A mouse. Proc Natl Acad Sci USA 86, 3070-4 (1989).
2. Forkert, P.G. Mechanisms of lung tumorigenesis by ethyl carbamate and vinyl carbamate. Drug Metab Rev 42, 355-78 (2010).
3. Westcott, P.M. et al. The mutational landscapes of genetic and chemical models of Kras-driven lung cancer. Nature 517, 489-92 (2015).
4. Nuzum, E.O., Malkinson, A.M. & Beer, D.G. Specific Ki-ras codon 61 mutations may determine the development of urethan-induced mouse lung adenomas or adenocarcinomas. Mol Carcinog 3, 287-95 (1990).

15. Dwyer-Nield, L.D. et al. Epistatic interactions govern chemically-induced lung tumor susceptibility and Kras mutation site in murine C57BL/6J-ChrA/J chromosome substitution strains. Int J Cancer 126, 125-32 (2010).
6. Cox, A.D., Fesik, S.W., Kimmelman, A.C., Luo, J. & Der, C.J. Drugging the undruggable RAS: Mission possible? Nat Rev Drug Discov 13, 828-51 (2014).
7. Prior, I.A., Lewis, P.D. & Mattos, C. A comprehensive survey of Ras mutations in cancer. Cancer Res 72, 2457-67 (2012).
8. Hernandez, L.G. & Forkert, P.G. In vivo mutagenicity of vinyl carbamate and ethyl carbamate in lung and small intestine of Fl (Big Blue x A/J) transgenic mice. Int J Cancer 120, 1426-33 (2007).
9. Fox, E.J., Reid-Bayliss, K.S., Emond, M.J. & Loeb, L.A. Accuracy of next generation sequencing platforms. Next Gener Seq Appl 1, 1000106 (2014).
10. Kinde, I., Wu, J., Papadopoulos, N., Kinzler, K.W. & Vogelstein, B. Detection and quantification of rare mutations with massively parallel sequencing. Proc Natl Acad Sci USA 108, 9530-5 (2011).
11. Schmitt, M.W. et al. Detection of ultra-rare mutations by next-generation sequencing. Proc Natl Acad Sci USA 109, 14508-13 (2012).
12. Lou, D.I. et al. High-throughput DNA sequencing errors are reduced by orders of magnitude using circle sequencing. Proc Natl Acad Sci USA 110, 19872-7 (2013).
13. Jee, J. et al. Rates and mechanisms of bacterial mutagenesis from maximum-depth sequencing. Nature 534, 693-6 (2016).
14. Blattner, F.R. et al. The complete genome sequence of Escherichia coli K-12. Science 277, 1453-62 (1997).
15. Mouse Genome Sequencing Corsortium. et al. Initial sequencing and comparative analysis of the mouse genome. Nature 420, 520-62 (2002).

### Example 3: Additional Primers for Maximum Depth Sequencing and Detection of Human KRAS Genes

Exon-specific barcode primer: [Forward
adaptor][Index][Barcode][ROI-specific
Primer] [Forward adpator] =
(SEQ ID NO: 1)
5′-TACGGCGACCACCGAGATCTACACTCTTTCCCTACACGACGCTCTTC
CGATCT-3′
[Index] = variable length of known sequences
from 0 to 7 nucleotides (specifically, 0
nucleotide, A, GA, CGA, TCGA, ATCGA, GATCGA or
CGATCGA) [Barcode] = 14 random nucleotides
(NNNNNNNNNNNNNN) [Primer] = Table 7 for KRAS exon
1 non-coding strand (first ROI specific sequence)
or Table 8 for KRAS exon 1 coding strand (first
ROI specific sequence)

TABLE 7
ROI-specific Primers for exon-specific
barcode primer (KRAS exon 1 non-coding
strand)

| | SEQ | |
|---|---|---|
| Restriction | ID | Primer sequence |
| enzyme | NO: | (primer annealing region) |
| StuI | 6 | 5′-CCTGCTGAAAATGACTGAA-3′ |
| PsiI | 18 | 5′-TAAGGCCTGCTGAAAATGA-3′ |
| Tsp45I | 19 | 5′-ATTTTCATTATTTTTATTATAAGG |
| | | CCTGC-3′ |
| AflIII or | 20 | 5′-TTCTAATATAGTCACATTTTCATT |
| PciI or | | ATTTTTATTATAAGG-3′ |
| FatI | | |
| NspI or | 21 | 5′-CATGTTCTAATATAGTCACATTTT |
| NlaIII | | CATTATTTT-3′ |
| CviAII | 22 | 5′-GTTCTAATATAGTCACATTTTCAT |
| | | TATTTTTATTATAAGG-3′ |
| CviQI | 23 | 5′-CTGGTGGAGTATTTGATAGTG-3′ |
| HphI | 24 | 5′-TTAAAAGGTACTGGTGGAGT-3′ |

TABLE 8
ROI-specific Primers for exon-specific
barcode primer (KRAS exon 1 coding strand)

| | SEQ | |
|---|---|---|
| Restriction | ID | |
| enzyme | NO: | Primer sequence |
| HinfI | 25 | 5′-CTGAATTAGCTGTATCGTCAAG-3′ |
| MluCI | 26 | 5′-AGCTGTATCGTCAAGGCA-3′ |
| Hpy188I | 27 | 5′-TGAATTAGCTGTATCGTCAAG-3′ |
| AlwI | 28 | 5′-CGTCCACAAAATGATTCTGA-3′ |
| DpnII | 29 | 5′-ATATTCGTCCACAAAATGATTCTG-3′ |
| MnlI | 30 | 5′-TGGATCATATTCGTCCACAA-3′ |
| NsiI | 31 | 5′-TGCATATTAAAACAAGATTTACCTCTAT-3′ |
| HpyCH4V | 32 | 5′-CATATTAAAACAAGATTTACCTCTATTGTT |
| | | G-3′ |
| BsrI | 33 | 5′-ACCAGTAATATGCATATTAAAACAAGA-3′ |

Forward-adaptor primer:
(SEQ ID NO: 2)
5′-AATGATACGGCGACCACCGAGAT-3′
The forward-adaptor primer binds the reverse
complement of SEQ ID NO: 1. It has a 5′ tail
of 5 bases AATGA that are added via
amplification and complete the adapter
sequence.
Exon-specific reverse primer: [Reverse
adaptor][Index][ROI-specific Primer]
[Reverse adaptor] =
(SEQ ID NO: 3)
5′-CAAGCAGAAGACGGCATACGAGATGTGACTGGAGTTCAGACGTGTGC
TCTTCCGATCT-3′
[Index] = variable length of known sequences from
0 to 7 nucleotides (specifically, 0 nucleotide, A,
GA, CGA, TCGA, ATCGA, GATCGA or CGATCGA) (Table 1)
[Primer] = Table 9

TABLE 9
Primers for exon-specific reverse primer
(KRAS exon 1) (second ROI-specific sequence)

| | SEQ ID | |
|---|---|---|
| Primer | NO: | Primer sequence |
| KRAS exon 1 | 8 | 5′-TAGCTGTATCGTCAAGGC-3′ |
| (non- coding | | |
| strand) | | |
| KRAS exon 1 | 17 | 5′-ATGACTGAATATAAACTTGTG |
| (coding strand) | | GTAGT-3′ |

| | Reverse-adaptor primer: |
|---|---|
| (SEQ ID NO: 4) | |
| | 5′-CAAGCAGAAGACGGCATACGAGA-3′ |

### Example 4: Detection of Mutations in uman Ras Genes Using Kras Exon 1 Primers in Human Blood

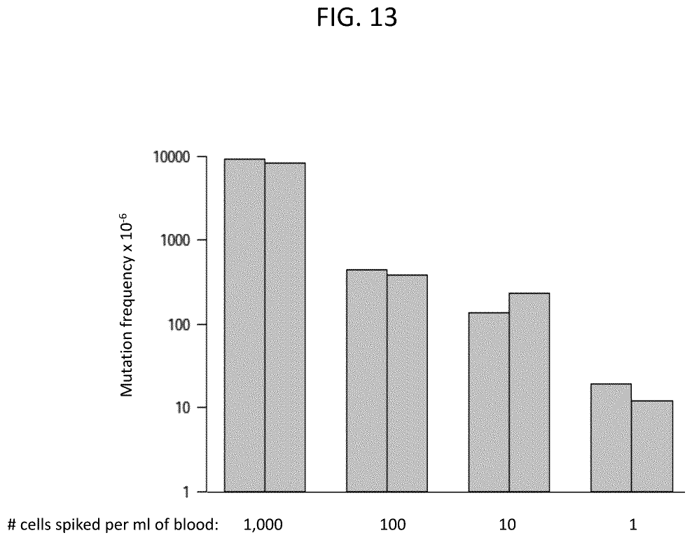

This example features the high-throughput sequence method using Kras exon 1 primers. These primers utilize a Hinfl restriction site. A barcode is added to any exon-specific primer. FIG. 13 shows the frequency of KRAS mutations identified using these primers. The results indicate that Kras exon 1 mutations can be detected to a frequency of as low as 1 cell/mL of human blood.

Exon-specific barcode primer: [Forward
adaptor][Index][Barcode][ROI-specific
Primer] [Forward adaptor] =
(SEQ ID NO: 1)
5′-TACGGCGACCACCGAGATCTACACTCTTTCCCTACACGACGCTCTTC
CGATCT-3′
[Index] = variable length of known sequences from
0 to 7 nucleotides [Barcode] = NNNNNNNNNNNNNN
[ROI-specific Primer] = Kras exon 1 Hinf1
Primer =
(SEQ ID NO: 9)
5′-CTGAATTAGCTGTATCGTCAAG-3′
(annealing temperature: 60° C.)
Forward-adaptor primer:
(SEQ ID NO: 2)
5′-AATGATACGGCGACCACCGAGAT-3′
(annealing temperature: 70° C.)
Exon-specific reverse primer: [Reverse adaptor +
barcode][Index][ROI-specific Primer]
[Reverse adaptor + barcode] =
(SEQ ID NO: 211)
5′-CAAGCAGAAGACGGCATACGAGATNNNNNNNNGTGACTGGAGTTCAG
ACGTGTGCTCTTCCGATCT-3′
[Index] = variable length of known sequences from
0 to 7 nucleotides
[ROI-specific Primer] Kras exon 1 Hinf1 =
(SEQ ID NO: 10)
5′-ATGACTGAATATAAACTTGTGGTAGT-3′
(annealing temperature: 62° C.)
Reverse-adaptor primer:
(SEQ ID NO: 4)
5′-CAAGCAGAAGACGGCATACGAGA-3′
(annealing temperature: 70° C.)

SEQUENCES - detection of human KRAS genes
Forward adaptor sequence
SEQ ID NO: 1
TACGGCGACCACCGAGATCTACACTCTTTCCCTACACGACGCTCTTCCGA
TCT
Forward adaptor primer
SEQ ID NO: 2
AATGATACGGCGACCACCGAGAT
Reverse adaptor sequence
SEQ ID NO: 3
CAAGCAGAAGACGGCATACGAGATGTGACTGGAGTTCAGACGTGTGCTCT
TCCGATCT
Reverse adaptor primer
SEQ ID NO: 4
CAAGCAGAAGACGGCATACGAGA
Generic barcode sequence
NNNNNNNNNNNNNN
First ROI-specific sequence for transcribed
strand of human KRAS exon 1
SEQ ID NO: 6
CCTGCTGAAAATGACTGAA
Second ROI-specific sequence for transcribed
strand of human KRAS exon 1
SEQ ID NO: 7
CTCTATTGTTGGATCATATTCGT
Alternative second ROI-specific sequence for
transcribed strand of human KRAS exon 1
SEQ ID NO: 8
TAGCTGTATCGTCAAGGC
First ROI-specific sequence for non-
ranscribed strand of human KRAS exon 1
SEQ ID NO: 9
CTGAATTAGCTGTATCGTCAAG
Second ROI-specific sequence for non-
transcribed strand of human KRAS exon 1
SEQ ID NO: 10
ATGACTGAATATAAACTTGTGGTAGT
First ROI-specific sequence for non-
transcribed strand of human KRAS exon 2
SEQ ID NO: 11
TCTTCAAATGATTTAGTATTATTTATGGC
Second ROI-specific sequence for non-
transcribed strand of human KRAS exon 2
SEQ ID NO: 12
GATTCCTACAGGAAGCAAGT
Exemplary ROI sequence shown in FIG. 1
SEQ ID NO: 13
AGGCCTGCTGAAAATGNAGGCCT
wherein N represents a region of undefined
sequence.
Exemplary ROI sequence shown in FIG. 1
SEQ ID NO: 14
TCCGGACGACTTTTACNTCCGGA
wherein N represents a region of undefined
sequence.
Exemplary ROI sequence shown in FIG. 1
SEQ ID NO: 15
CCTGCTGAAAATGNAGG
wherein N represents a region of undefined
sequence.
Exemplary ROI sequence shown in FIG. 1
SEQ ID NO: 16
GGACGACTTTTACNTCC
wherein N represents a region of undefined
sequence.
Full-length adaptor-barcode primer for
transcribed strand of Kras exon 1
SEQ ID NO: 34
TACGGCGACCACCGAGATCTACACTCTTTCCCTACACGACGCTCTTCCGA
TCTNNNNNNNNNNNNNNCCTGCTGAAAATGACTGAA
wherein N represents a region of undefined
sequence.
Full-length exon-specific reverse primer
for transcribed strand of Kras exon 1
SEQ ID NO: 35
CAAGCAGAAGACGGCATACGAGATGTGACTGGAGTTCAGACGTGTGCTCT
TCCGATCTCTCTATTGTTGGATCATATTCGT
Alternative full-length exon-specific
reverse primer for transcribed strand
of Kras exon 1
SEQ ID NO: 36
CAAGCAGAAGACGGCATACGAGATGTGACTGGAGTTCAGACGTGTGCTCT
TCCGATCTTAGCTGTATCGTCAAGGC
Full-length adaptor-barcode primer for
non-transcribed strand of Kras exon 1
SEQ ID NO: 37
TACGGCGACCACCGAGATCTACACTCTTTCCCTACACGACGCTCTTCCGA
TCTNNNNNNNNNNNNNNCTGAATTAGCTGTATCGTCAAG
wherein N represents a region of undefined
sequence.
Full-length exon-specific reverse primer for
non-transcribed strand of Kras exon 1
SEQ ID NO: 38
CAAGCAGAAGACGGCATACGAGATGTGACTGGAGTTCAGACGTGTGCTCT
TCCGATCTATGACTGAATATAAACTTGTGGTAGT
Full-length adaptor-barcode primer for non-
transcribed strand of Kras exon 2
SEQ ID NO: 39
TACGGCGACCACCGAGATCTACACTCTTTCCCTACACGACGCTCTTCCGA
TCTNNNNNNNNNNNNNNTCTTCAAATGATTTAGTATTATTTATGGC
wherein N represents a region of undefined
sequence.
Full-length exon-specific reverse primer for
non-transcribed strand of Kras exon 2
SEQ ID NO: 40
CAAGCAGAAGACGGCATACGAGATGTGACTGGAGTTCAGACGTGTGCTCT
TCCGATCTGATTCCTACAGGAAGCAAGT

TABLE 10
Full-length adaptor-barcode primers for KRAS coding strand

| Restriction | Sequence (5′→3′: forward adaptor sequence, index, |
|---|---|
| Enzyme | barcode, first ROI-specific sequence) |
| HinfI | TACGGCGACCACCGAGATCTACACTCTTTCCCTACACGACGCTCTTCC |
| | GATCTNNNNNNNNNNNNNNCTGAATTAGCTGTATCGTCAAG (SEQ ID |
| | NO: 37) |
| HinfI | TACGGCGACCACCGAGATCTACACTCTTTCCCTACACGACGCTCTTCC |
| | GATCTANNNNNNNNNNNNNNCTGAATTAGCTGTATCGTCAAG (SEQ |
| | ID NO: 41) |
| HinfI | TACGGCGACCACCGAGATCTACACTCTTTCCCTACACGACGCTCTTCC |
| | GATCTGANNNNNNNNNNNNNNCTGAATTAGCTGTATCGTCAAG |
| | (SEQ ID NO: 42) |
| HinfI | TACGGCGACCACCGAGATCTACACTCTTTCCCTACACGACGCTCTTCC |
| | GATCTCGANNNNNNNNNNNNNNCTGAATTAGCTGTATCGTCAAG |
| | (SEQ ID NO: 43) |
| HinfI | TACGGCGACCACCGAGATCTACACTCTTTCCCTACACGACGCTCTTCC |
| | GATCTTCGANNNNNNNNNNNNNNCTGAATTAGCTGTATCGTCAAG |
| | (SEQ ID NO: 44) |
| HinfI | TACGGCGACCACCGAGATCTACACTCTTTCCCTACACGACGCTCTTCC |
| | GATCTATCGANNNNNNNNNNNNNNCTGAATTAGCTGTATCGTCAAG |
| | (SEQ ID NO: 45) |
| HinfI | TACGGCGACCACCGAGATCTACACTCTTTCCCTACACGACGCTCTTCC |
| | GATCTGATCGANNNNNNNNNNNNNNCTGAATTAGCTGTATCGTCAAG |
| | (SEQ ID NO: 46) |
| HinfI | TACGGCGACCACCGAGATCTACACTCTTTCCCTACACGACGCTCTTCC |
| | GATCTCGATCGANNNNNNNNNNNNNNCTGAATTAGCTGTATCGTCAA |
| | G (SEQ ID NO: 47) |
| AlwI | TACGGCGACCACCGAGATCTACACTCTTTCCCTACACGACGCTCTTCC |
| | GATCTNNNNNNNNNNNNNNcgtccacaaaatgattctga (SEQ ID NO: 48) |
| AlwI | TACGGCGACCACCGAGATCTACACTCTTTCCCTACACGACGCTCTTCC |
| | GATCTANNNNNNNNNNNNNNcgtccacaaaatgattctga (SEQ ID NO: 49) |
| AlwI | TACGGCGACCACCGAGATCTACACTCTTTCCCTACACGACGCTCTTCC |
| | GATCTGANNNNNNNNNNNNNNcgtccacaaaatgattctga (SEQ ID NO: 50) |
| AlwI | TACGGCGACCACCGAGATCTACACTCTTTCCCTACACGACGCTCTTCC |
| | GATCTCGANNNNNNNNNNNNNNcgtccacaaaatgattctga (SEQ ID NO: 51) |
| AlwI | TACGGCGACCACCGAGATCTACACTCTTTCCCTACACGACGCTCTTCC |
| | GATCTTCGANNNNNNNNNNNNNNcgtccacaaaatgattctga (SEQ ID NO: 52) |
| AlwI | TACGGCGACCACCGAGATCTACACTCTTTCCCTACACGACGCTCTTCC |
| | GATCTATCGANNNNNNNNNNNNNNcgtccacaaaatgattctga (SEQ ID NO: 53) |
| AlwI | TACGGCGACCACCGAGATCTACACTCTTTCCCTACACGACGCTCTTCC |
| | GATCTGATCGANNNNNNNNNNNNNNcgtccacaaaatgattctga (SEQ ID NO: |
| | 54) |
| AlwI | TACGGCGACCACCGAGATCTACACTCTTTCCCTACACGACGCTCTTCC |
| | GATCTCGATCGANNNNNNNNNNNNNNcgtccacaaaatgattctga (SEQ ID NO: |
| | 55) |
| BsrI | TACGGCGACCACCGAGATCTACACTCTTTCCCTACACGACGCTCTTCCGAT |
| | CTNNNNNNNNNNNNNNaccagtaatatgcatattaaaacaaga (SEQ ID NO: 56) |
| BsrI | TACGGCGACCACCGAGATCTACACTCTTTCCCTACACGACGCTCTTCC |
| | GATCTANNNNNNNNNNNNNNaccagtaatatgcatattaaaacaaga (SEQ ID NO: |
| | 57) |
| BsrI | TACGGCGACCACCGAGATCTACACTCTTTCCCTACACGACGCTCTTCC |
| | GATCTGANNNNNNNNNNNNNNaccagtaatatgcatattaaaacaaga (SEQ ID NO: |
| | 58) |
| BsrI | TACGGCGACCACCGAGATCTACACTCTTTCCCTACACGACGCTCTTCC |
| | GATCTCGANNNNNNNNNNNNNNaccagtaatatgcatattaaaacaaga (SEQ |
| | ID NO: 59) |
| BsrI | TACGGCGACCACCGAGATCTACACTCTTTCCCTACACGACGCTCTTCC |
| | GATCTTCGANNNNNNNNNNNNNNaccagtaatatgcatattaaaacaaga (SEQ ID |
| | NO: 60) |
| BsrI | TACGGCGACCACCGAGATCTACACTCTTTCCCTACACGACGCTCTTCC |
| | GATCTATCGANNNNNNNNNNNNNNaccagtaatatgcatattaaaacaaga (SEQ ID |
| | NO: 61) |
| BsrI | TACGGCGACCACCGAGATCTACACTCTTTCCCTACACGACGCTCTTCC |
| | GATCTGATCGANNNNNNNNNNNNNNaccagtaatatgcatattaaaacaaga (SEQ |
| | ID NO: 62) |
| BsrI | TACGGCGACCACCGAGATCTACACTCTTTCCCTACACGACGCTCTTCC |
| | GATCTCGATCGANNNNNNNNNNNNNNaccagtaatatgcatattaaaacaaga (SEQ |
| | ID NO: 63) |
| DpnII | TACGGCGACCACCGAGATCTACACTCTTTCCCTACACGACGCTCTTCC |
| | GATCTNNNNNNNNNNNNNNatattcgtccacaaaatgattctg (SEQ ID NO: 64) |
| DpnII | TACGGCGACCACCGAGATCTACACTCTTTCCCTACACGACGCTCTTCC |
| | GATCTANNNNNNNNNNNNNNatattegtccacaaaatgattctg (SEQ ID NO: 65) |
| DpnII | TACGGCGACCACCGAGATCTACACTCTTTCCCTACACGACGCTCTTCC |
| | GATCTGANNNNNNNNNNNNNNatattcgtccacaaaatgattctg (SEQ ID NO: 66) |
| DpnII | TACGGCGACCACCGAGATCTACACTCTTTCCCTACACGACGCTCTTCCGAT |
| | CTCGANNNNNNNNNNNNNNatattcgtccacaaaatgattctg (SEQ ID NO: 67) |
| DpnII | TACGGCGACCACCGAGATCTACACTCTTTCCCTACACGACGCTCTTCC |
| | GATCTTCGANNNNNNNNNNNNNNatattcgtccacaaaatgattctg (SEQ ID NO: |
| | 68) |
| DpnII | TACGGCGACCACCGAGATCTACACTCTTTCCCTACACGACGCTCTTCC |
| | GATCTATCGANNNNNNNNNNNNNNatattcgtccacaaaatgattctg (SEQ ID NO: |
| | 69) |
| DpnII | TACGGCGACCACCGAGATCTACACTCTTTCCCTACACGACGCTCTTCC |
| | GATCTGATCGANNNNNNNNNNNNNNatattcgtccacaaaatgattctg (SEQ ID |
| | NO: 70) |
| DpnII | TACGGCGACCACCGAGATCTACACTCTTTCCCTACACGACGCTCTTCC |
| | GATCTCGATCGANNNNNNNNNNNNNNatattcgtccacaaaatgattctg (SEQ ID |
| | NO: 71) |
| Hpy188I | TACGGCGACCACCGAGATCTACACTCTTTCCCTACACGACGCTCTTCC |
| | GATCTNNNNNNNNNNNNNNtgaattagctgtatcgtcaag (SEQ ID NO: 72) |
| Hpy188I | TACGGCGACCACCGAGATCTACACTCTTTCCCTACACGACGCTCTTCC |
| | GATCTANNNNNNNNNNNNNNtgaattagctgtatcgtcaag (SEQ ID NO: 73) |
| Hpy188I | TACGGCGACCACCGAGATCTACACTCTTTCCCTACACGACGCTCTTCC |
| | GATCTGANNNNNNNNNNNNNNtgaattagctgtatcgtcaag (SEQ ID NO: 74) |
| Hpy188I | TACGGCGACCACCGAGATCTACACTCTTTCCCTACACGACGCTCTTCC |
| | GATCTCGANNNNNNNNNNNNNNtgaattagctgtatcgtcaag (SEQ ID NO: 75) |
| Hpy188I | TACGGCGACCACCGAGATCTACACTCTTTCCCTACACGACGCTCTTCC |
| | GATCTTCGANNNNNNNNNNNNNNtgaattagctgtatcgtcaag (SEQ ID NO: 76) |
| Hpy188I | TACGGCGACCACCGAGATCTACACTCTTTCCCTACACGACGCTCTTCC |
| | GATCTATCGANNNNNNNNNNNNNNtgaattagctgtatcgtcaag (SEQ ID NO: |
| | 77) |
| Hpy188I | TACGGCGACCACCGAGATCTACACTCTTTCCCTACACGACGCTCTTCC |
| | GATCTGATCGANNNNNNNNNNNNNNtgaattagctgtatcgtcaag (SEQ ID NO: |
| | 78) |
| Hpy188I | TACGGCGACCACCGAGATCTACACTCTTTCCCTACACGACGCTCTTCC |
| | GATCTCGATCGANNNNNNNNNNNNNNtgaattagctgtatcgtcaag (SEQ ID NO: |
| | 79) |
| HpyCH4V | TACGGCGACCACCGAGATCTACACTCTTTCCCTACACGACGCTCTTCC |
| | GATCTNNNNNNNNNNNNNNcatattaaaacaagatttacctctattgttg |
| | (SEQ ID NO: 80) |
| HpyCH4V | TACGGCGACCACCGAGATCTACACTCTTTCCCTACACGACGCTCTTCC |
| | GATCTANNNNNNNNNNNNNNcatattaaaacaagatttacctctattgttg |
| | (SEQ ID NO: 81) |
| HpyCH4V | TACGGCGACCACCGAGATCTACACTCTTTCCCTACACGACGCTCTTCC |
| | GATCTGANNNNNNNNNNNNNNcatattaaaacaagatttacctctattgttg (SEQ ID |
| | NO: 82) |
| HpyCH4V | TACGGCGACCACCGAGATCTACACTCTTTCCCTACACGACGCTCTTCC |
| | GATCTCGANNNNNNNNNNNNNNcatattaaaacaagatttacctctattgttg |
| | (SEQ ID NO: 83) |
| HpyCH4V | TACGGCGACCACCGAGATCTACACTCTTTCCCTACACGACGCTCTTCC |
| | GATCTTCGANNNNNNNNNNNNNNcatattaaaacaagatttacctctattgttg |
| | (SEQ ID NO: 84) |
| HpyCH4V | TACGGCGACCACCGAGATCTACACTCTTTCCCTACACGACGCTCTTCC |
| | GATCTATCGANNNNNNNNNNNNNNcatattaaaacaagatttacctctattgttg |
| | (SEQ ID NO: 85) |
| HpyCH4V | TACGGCGACCACCGAGATCTACACTCTTTCCCTACACGACGCTCTTCC |
| | GATCTGATCGANNNNNNNNNNNNNNcatattaaaacaagatttacctctattgttg |
| | (SEQ ID NO: 86) |
| HpyCH4V | TACGGCGACCACCGAGATCTACACTCTTTCCCTACACGACGCTCTTCC |
| | GATCTCGATCGANNNNNNNNNNNNNNcatattaaaacaagatttacctctattgttg |
| | (SEQ ID NO: 87) |
| MluCI | TACGGCGACCACCGAGATCTACACTCTTTCCCTACACGACGCTCTTCC |
| | GATCTNNNNNNNNNNNNNNagctgtatcgtcaaggca (SEQ ID NO: 88) |
| MluCI | TACGGCGACCACCGAGATCTACACTCTTTCCCTACACGACGCTCTTCC |
| | GATCTANNNNNNNNNNNNNNagctgtatcgtcaaggca (SEQ ID NO: 89) |
| MluCI | TACGGCGACCACCGAGATCTACACTCTTTCCCTACACGACGCTCTTCC |
| | GATCTGANNNNNNNNNNNNNNagctgtatcgtcaaggca (SEQ ID NO: 90) |
| MluCI | TACGGCGACCACCGAGATCTACACTCTTTCCCTACACGACGCTCTTCC |
| | GATCTCGANNNNNNNNNNNNNNagctgtatcgtcaaggca (SEQ ID NO: 91) |
| MluCI | TACGGCGACCACCGAGATCTACACTCTTTCCCTACACGACGCTCTTCC |
| | GATCTTCGANNNNNNNNNNNNNNagctgtatcgtcaaggca (SEQ ID NO: 92) |
| MluCI | TACGGCGACCACCGAGATCTACACTCTTTCCCTACACGACGCTCTTCC |
| | GATCTATCGANNNNNNNNNNNNNNagctgtatcgtcaaggca (SEQ ID NO: 93) |
| MluCI | TACGGCGACCACCGAGATCTACACTCTTTCCCTACACGACGCTCTTCC |
| | GATCTGATCGANNNNNNNNNNNNNNagctgtatcgtcaaggca (SEQ ID NO: |
| | 94) |
| MluCI | TACGGCGACCACCGAGATCTACACTCTTTCCCTACACGACGCTCTTCC |
| | GATCTCGATCGANNNNNNNNNNNNNNagctgtatcgtcaaggca (SEQ ID NO: |
| | 95) |
| MnlI | TACGGCGACCACCGAGATCTACACTCTTTCCCTACACGACGCTCTTCC |
| | GATCTNNNNNNNNNNNNNNtggatcatattcgtccacaa (SEQ ID NO: 96) |
| MnlI | TACGGCGACCACCGAGATCTACACTCTTTCCCTACACGACGCTCTTCC |
| | GATCTANNNNNNNNNNNNNNtggatcatattcgtccacaa (SEQ ID NO: 97) |
| MnlI | TACGGCGACCACCGAGATCTACACTCTTTCCCTACACGACGCTCTTCC |
| | GATCTGANNNNNNNNNNNNNNtggatcatattcgtccacaa (SEQ ID NO: 98) |
| MnlI | TACGGCGACCACCGAGATCTACACTCTTTCCCTACACGACGCTCTTCC |
| | GATCTCGANNNNNNNNNNNNNNtggatcatattcgtccacaa (SEQ ID NO: 99) |
| MnlI | TACGGCGACCACCGAGATCTACACTCTTTCCCTACACGACGCTCTTCC |
| | GATCTTCGANNNNNNNNNNNNNNtggatcatattcgtccacaa (SEQ ID NO: 100) |
| MnlI | TACGGCGACCACCGAGATCTACACTCTTTCCCTACACGACGCTCTTCC |
| | GATCTATCGANNNNNNNNNNNNNNtggatcatattcgtccacaa (SEQ ID NO: |
| | 101) |
| MnlI | TACGGCGACCACCGAGATCTACACTCTTTCCCTACACGACGCTCTTCC |
| | GATCTGATCGANNNNNNNNNNNNNNtggatcatattcgtccacaa (SEQ ID NO: |
| | 102) |
| MnlI | TACGGCGACCACCGAGATCTACACTCTTTCCCTACACGACGCTCTTCC |
| | GATCTCGATCGANNNNNNNNNNNNNNtggatcatattcgtccacaa (SEQ ID NO: |
| | 103) |
| NsiI | TACGGCGACCACCGAGATCTACACTCTTTCCCTACACGACGCTCTTCCGAT |
| | CTNNNNNNNNNNNNNNtgcatattaaaacaagatttacctctat (SEQ ID NO: 104) |
| NsiI | TACGGCGACCACCGAGATCTACACTCTTTCCCTACACGACGCTCTTCC |
| | GATCTANNNNNNNNNNNNNNtgcatattaaaacaagatttacctctat (SEQ ID NO: |
| | 105) |
| NsiI | TACGGCGACCACCGAGATCTACACTCTTTCCCTACACGACGCTCTTCC |
| | GATCTGANNNNNNNNNNNNNNtgcatattaaaacaagatttacctctat |
| | (SEQ ID NO: 106) |
| NsiI | TACGGCGACCACCGAGATCTACACTCTTTCCCTACACGACGCTCTTCC |
| | GATCTCGANNNNNNNNNNNNNNtgcatattaaaacaagatttacctctat (SEQ ID |
| | NO: 107) |
| NsiI | TACGGCGACCACCGAGATCTACACTCTTTCCCTACACGACGCTCTTCC |
| | GATCTTCGANNNNNNNNNNNNNNtgcatattaaaacaagatttacctctat (SEQ ID |
| | NO: 108) |
| NsiI | TACGGCGACCACCGAGATCTACACTCTTTCCCTACACGACGCTCTTCC |
| | GATCTATCGANNNNNNNNNNNNNNtgcatattaaaacaagatttacctctat (SEQ ID |
| | NO: 109) |
| NsiI | TACGGCGACCACCGAGATCTACACTCTTTCCCTACACGACGCTCTTCC |
| | GATCTGATCGANNNNNNNNNNNNNNtgcatattaaaacaagatttacctctat (SEQ |
| | ID NO: 110) |
| NsiI | TACGGCGACCACCGAGATCTACACTCTTTCCCTACACGACGCTCTTCC |
| | GATCTCGATCGANNNNNNNNNNNNNNtgcatattaaaacaagatttacctctat (SEQ |
| | ID NO: 111) |

TABLE 11
Full-length exon-specific reverse primers
for KRAS coding strand
Sequence (5′→3′: reverse adaptor sequence,
index, second ROI-specific sequence)

CAAGCAGAAGACGGCATACGAGATGTGACTGGAGTTCAGACGTGTGCTCTTCCGA
TCTATGACTGAATATAAACTTGTGGTAGT (SEQ ID NO: 38)
CAAGCAGAAGACGGCATACGAGATGTGACTGGAGTTCAGACGTGTGCTCTTCCGA
TCTAATGACTGAATATAAACTTGTGGTAGT (SEQ ID NO: 112)
CAAGCAGAAGACGGCATACGAGATGTGACTGGAGTTCAGACGTGTGCTCTTCCGA
TCTGAATGACTGAATATAAACTTGTGGTAGT (SEQ ID NO: 113)
CAAGCAGAAGACGGCATACGAGATGTGACTGGAGTTCAGACGTGTGCTCTTCCGA
TCTCGAATGACTGAATATAAACTTGTGGTAGT (SEQ ID NO: 114)
CAAGCAGAAGACGGCATACGAGATGTGACTGGAGTTCAGACGTGTGCTCTTCCGA
TCTTCGAATGACTGAATATAAACTTGTGGTAGT (SEQ ID NO: 115)
CAAGCAGAAGACGGCATACGAGATGTGACTGGAGTTCAGACGTGTGCTCTTCCGA
TCTATCGAATGACTGAATATAAACTTGTGGTAGT (SEQ ID NO: 116)
CAAGCAGAAGACGGCATACGAGATGTGACTGGAGTTCAGACGTGTGCTCTTCCGA
TCTGATCGAATGACTGAATATAAACTTGTGGTAGT (SEQ ID NO: 117)
CAAGCAGAAGACGGCATACGAGATGTGACTGGAGTTCAGACGTGTGCTCTTCCGA
TCTCGATCGAATGACTGAATATAAACTTGTGGTAGT (SEQ ID NO: 118)

TABLE 12
Full-length adaptor-barcode primers for KRAS non-coding strand

| Restriction | Sequence (5′→3′: forward adaptor sequence, index, |
|---|---|
| Enzyme | barcode, first ROI-specific sequence) |
| StuI | TACGGCGACCACCGAGATCTACACTCTTTCCCTACACGACGCTCTTCC |
| | GATCTNNNNNNNNNNNNNNCCTGCTGAAAATGACTGAA (SEQ ID NO: |
| | 34) |
| StuI | TACGGCGACCACCGAGATCTACACTCTTTCCCTACACGACGCTCTTCC |
| | GATCTANNNNNNNNNNNNNNCCTGCTGAAAATGACTGAA (SEQ ID |
| | NO: 119) |
| StuI | TACGGCGACCACCGAGATCTACACTCTTTCCCTACACGACGCTCTTCC |
| | GATCTGANNNNNNNNNNNNNNCCTGCTGAAAATGACTGAA (SEQ ID |
| | NO: 120) |
| StuI | TACGGCGACCACCGAGATCTACACTCTTTCCCTACACGACGCTCTTCC |
| | GATCTCGANNNNNNNNNNNNNNCCTGCTGAAAATGACTGAA (SEQ ID |
| | NO: 121) |
| StuI | TACGGCGACCACCGAGATCTACACTCTTTCCCTACACGACGCTCTTCC |
| | GATCTTCGANNNNNNNNNNNNNNCCTGCTGAAAATGACTGAA (SEQ |
| | ID NO: 122) |
| StuI | TACGGCGACCACCGAGATCTACACTCTTTCCCTACACGACGCTCTTCC |
| | GATCTATCGANNNNNNNNNNNNNNCCTGCTGAAAATGACTGAA (SEQ |
| | ID NO: 123) |
| StuI | TACGGCGACCACCGAGATCTACACTCTTTCCCTACACGACGCTCTTCC |
| | GATCTGATCGANNNNNNNNNNNNNNCCTGCTGAAAATGACTGAA |
| | (SEQ ID NO: 124) |
| StuI | TACGGCGACCACCGAGATCTACACTCTTTCCCTACACGACGCTCTTCC |
| | GATCTCGATCGANNNNNNNNNNNNNNCCTGCTGAAAATGACTGAA |
| | (SEQ ID NO: 125) |
| AflIII | TACGGCGACCACCGAGATCTACACTCTTTCCCTACACGACGCTCTTCC |
| | GATCTNNNNNNNNNNNNNNttctaatatagtcacattttcattatttttattataagg (SEQ ID |
| | NO: 126) |
| AflIII | TACGGCGACCACCGAGATCTACACTCTTTCCCTACACGACGCTCTTCC |
| | GATCTANNNNNNNNNNNNNNttctaatatagtcacattttcattatttttattataagg |
| | (SEQ ID NO: 127) |
| AflIII | TACGGCGACCACCGAGATCTACACTCTTTCCCTACACGACGCTCTTCC |
| | GATCTGANNNNNNNNNNNNNNttctaatatagtcacattttcattatttttattataagg (SEQ |
| | ID NO: 128) |
| AflIII | TACGGCGACCACCGAGATCTACACTCTTTCCCTACACGACGCTCTTCC |
| | GATCTCGANNNNNNNNNNNNNNttctaatatagtcacattttcattatttttattataagg (SEQ |
| | ID NO: 129) |
| AflIII | TACGGCGACCACCGAGATCTACACTCTTTCCCTACACGACGCTCTTCC |
| | GATCTTCGANNNNNNNNNNNNNNttctaatatagtcacattttcattatttttattataagg |
| | (SEQ ID NO: 130) |
| AflIII | TACGGCGACCACCGAGATCTACACTCTTTCCCTACACGACGCTCTTCC |
| | GATCTATCGANNNNNNNNNNNNNNttctaatatagtcacattttcattatttttattataagg |
| | (SEQ ID NO: 131) |
| AflIII | TACGGCGACCACCGAGATCTACACTCTTTCCCTACACGACGCTCTTCC |
| | GATCTGATCGANNNNNNNNNNNNNNttctaatatagtcacattttcattatttttattataagg |
| | (SEQ ID NO: 132) |
| AflIII | TACGGCGACCACCGAGATCTACACTCTTTCCCTACACGACGCTCTTCC |
| | GATCTCGATCGANNNNNNNNNNNNNNttctaatatagtcacattttcattatttttattataagg |
| | (SEQ ID NO: 133) |
| CviAII | TACGGCGACCACCGAGATCTACACTCTTTCCCTACACGACGCTCTTCC |
| | GATCTNNNNNNNNNNNNNNgttctaatatagtcacattttcattatttttattataagg |
| | (SEQ ID NO: 134) |
| CviAII | TACGGCGACCACCGAGATCTACACTCTTTCCCTACACGACGCTCTTCC |
| | GATCTANNNNNNNNNNNNNNgttctaatatagtcacattttcattatttttattataagg (SEQ |
| | ID NO: 135) |
| CviAII | TACGGCGACCACCGAGATCTACACTCTTTCCCTACACGACGCTCTTCC |
| | GATCTGANNNNNNNNNNNNNNgttctaatatagtcacattttcattatttttattataagg (SEQ |
| | ID NO: 136) |
| CviAII | TACGGCGACCACCGAGATCTACACTCTTTCCCTACACGACGCTCTTCC |
| | GATCTCGANNNNNNNNNNNNNNgttctaatatagtcacattttcattatttttattataagg |
| | (SEQ ID NO: 137) |
| CviAII | TACGGCGACCACCGAGATCTACACTCTTTCCCTACACGACGCTCTTCC |
| | GATCTTCGANNNNNNNNNNNNNNgttctaatatagtcacattttcattatttttattataagg |
| | (SEQ ID NO: 138) |
| CviAII | TACGGCGACCACCGAGATCTACACTCTTTCCCTACACGACGCTCTTCC |
| | GATCTATCGANNNNNNNNNNNNNNgttctaatatagtcacattttcattatttttattataagg |
| | (SEQ ID NO: 139) |
| CviAII | TACGGCGACCACCGAGATCTACACTCTTTCCCTACACGACGCTCTTCC |
| | GATCTGATCGANNNNNNNNNNNNNNgttctaatatagtcacattttcattatttttattataagg |
| | (SEQ ID NO: 140) |
| CviAII | TACGGCGACCACCGAGATCTACACTCTTTCCCTACACGACGCTCTTCC |
| | GATCTCGATCGANNNNNNNNNNNNNNgttctaatatagtcacattttcattatttttattataag |
| | g (SEQ ID NO: 141) |
| CviQI | TACGGCGACCACCGAGATCTACACTCTTTCCCTACACGACGCTCTTCC |
| | GATCTNNNNNNNNNNNNNNctggtggagtatttgatagtg (SEQ ID NO: 142) |
| CviQI | TACGGCGACCACCGAGATCTACACTCTTTCCCTACACGACGCTCTTCC |
| | GATCTANNNNNNNNNNNNNNctggtggagtatttgatagtg (SEQ ID NO: 143) |
| CviQI | TACGGCGACCACCGAGATCTACACTCTTTCCCTACACGACGCTCTTCC |
| | GATCTGANNNNNNNNNNNNNNctggtggagtatttgatagtg (SEQ ID NO: 144) |
| CviQI | TACGGCGACCACCGAGATCTACACTCTTTCCCTACACGACGCTCTTCC |
| | GATCTCGANNNNNNNNNNNNNNctggtggagtatttgatagtg (SEQ ID NO: 145) |
| CviQI | TACGGCGACCACCGAGATCTACACTCTTTCCCTACACGACGCTCTTCC |
| | GATCTTCGANNNNNNNNNNNNNNctggtggagtatttgatagtg (SEQ ID NO: 146) |
| CviQI | TACGGCGACCACCGAGATCTACACTCTTTCCCTACACGACGCTCTTCC |
| | GATCTATCGANNNNNNNNNNNNNNctggtggagtatttgatagtg (SEQ ID NO: |
| | 147) |
| CviQI | TACGGCGACCACCGAGATCTACACTCTTTCCCTACACGACGCTCTTCC |
| | GATCTGATCGANNNNNNNNNNNNNNctggtggagtatttgatagtg (SEQ ID NO: |
| | 148) |
| CviQI | TACGGCGACCACCGAGATCTACACTCTTTCCCTACACGACGCTCTTCC |
| | GATCTCGATCGANNNNNNNNNNNNNNctggtggagtatttgatagtg (SEQ ID NO: |
| | 149) |
| HphI | TACGGCGACCACCGAGATCTACACTCTTTCCCTACACGACGCTCTTCC |
| | GATCTNNNNNNNNNNNNNNttaaaaggtactggtggagt (SEQ ID NO: 150) |
| HphI | TACGGCGACCACCGAGATCTACACTCTTTCCCTACACGACGCTCTTCC |
| | GATCTANNNNNNNNNNNNNNttaaaaggtactggtggagt (SEQ ID NO: 151) |
| HphI | TACGGCGACCACCGAGATCTACACTCTTTCCCTACACGACGCTCTTCC |
| | GATCTGANNNNNNNNNNNNNNttaaaaggtactggtggagt (SEQ ID NO: 152) |
| HphI | TACGGCGACCACCGAGATCTACACTCTTTCCCTACACGACGCTCTTCC |
| | GATCTCGANNNNNNNNNNNNNNttaaaaggtactggtggagt (SEQ ID NO: 153) |
| HphI | TACGGCGACCACCGAGATCTACACTCTTTCCCTACACGACGCTCTTCC |
| | GATCTTCGANNNNNNNNNNNNNNttaaaaggtactggtggagt (SEQ ID NO: 154) |
| HphI | TACGGCGACCACCGAGATCTACACTCTTTCCCTACACGACGCTCTTCC |
| | GATCTATCGANNNNNNNNNNNNNNttaaaaggtactggtggagt (SEQ ID NO: |
| | 155) |
| HphI | TACGGCGACCACCGAGATCTACACTCTTTCCCTACACGACGCTCTTCC |
| | GATCTGATCGANNNNNNNNNNNNNNttaaaaggtactggtggagt (SEQ ID NO: |
| | 156) |
| HphI | TACGGCGACCACCGAGATCTACACTCTTTCCCTACACGACGCTCTTCC |
| | GATCTCGATCGANNNNNNNNNNNNNNttaaaaggtactggtggagt (SEQ ID NO: |
| | 157) |
| NspI | TACGGCGACCACCGAGATCTACACTCTTTCCCTACACGACGCTCTTCC |
| | GATCTNNNNNNNNNNNNNNcatgttctaatatagtcacattttcattatttt (SEQ ID NO: |
| | 158) |
| NspI | TACGGCGACCACCGAGATCTACACTCTTTCCCTACACGACGCTCTTCC |
| | GATCTANNNNNNNNNNNNNNcatgttctaatatagtcacattttcattatttt (SEQ ID NO: |
| | 159) |
| NspI | TACGGCGACCACCGAGATCTACACTCTTTCCCTACACGACGCTCTTCC |
| | GATCTGANNNNNNNNNNNNNNcatgttctaatatagtcacattttcattatttt (SEQ ID |
| | NO: 160) |
| NspI | TACGGCGACCACCGAGATCTACACTCTTTCCCTACACGACGCTCTTCC |
| | GATCTCGANNNNNNNNNNNNNNcatgttctaatatagtcacattttcattatttt (SEQ ID |
| | NO: 161) |
| NspI | TACGGCGACCACCGAGATCTACACTCTTTCCCTACACGACGCTCTTCC |
| | GATCTTCGANNNNNNNNNNNNNNcatgttctaatatagtcacattttcattatttt (SEQ ID |
| | NO: 162) |
| NspI | TACGGCGACCACCGAGATCTACACTCTTTCCCTACACGACGCTCTTCC |
| | GATCTATCGANNNNNNNNNNNNNNcatgttctaatatagtcacattttcattatttt (SEQ |
| | ID NO: 163) |
| NspI | TACGGCGACCACCGAGATCTACACTCTTTCCCTACACGACGCTCTTCC |
| | GATCTGATCGANNNNNNNNNNNNNNcatgttctaatatagtcacattttcattatttt (SEQ |
| | ID NO: 164) |
| NspI | TACGGCGACCACCGAGATCTACACTCTTTCCCTACACGACGCTCTTCC |
| | GATCTCGATCGANNNNNNNNNNNNNNcatgttctaatatagtcacattttcattatttt |
| | (SEQ ID NO: 165) |
| PsiI | TACGGCGACCACCGAGATCTACACTCTTTCCCTACACGACGCTCTTCC |
| | GATCTNNNNNNNNNNNNNNtaaggcctgctgaaaatga (SEQ ID NO: 166) |
| PsiI | TACGGCGACCACCGAGATCTACACTCTTTCCCTACACGACGCTCTTCC |
| | GATCTANNNNNNNNNNNNNNtaaggcctgctgaaaatga (SEQ ID NO: 167) |
| PsiI | TACGGCGACCACCGAGATCTACACTCTTTCCCTACACGACGCTCTTCC |
| | GATCTGANNNNNNNNNNNNNNtaaggcctgctgaaaatga (SEQ ID NO: 168) |
| PsiI | TACGGCGACCACCGAGATCTACACTCTTTCCCTACACGACGCTCTTCC |
| | GATCTCGANNNNNNNNNNNNNNtaaggcctgctgaaaatga (SEQ ID NO: 169) |
| PsiI | TACGGCGACCACCGAGATCTACACTCTTTCCCTACACGACGCTCTTCC |
| | GATCTTCGANNNNNNNNNNNNNNtaaggcctgctgaaaatga (SEQ ID NO: 170) |
| PsiI | TACGGCGACCACCGAGATCTACACTCTTTCCCTACACGACGCTCTTCC |
| | GATCTATCGANNNNNNNNNNNNNNtaaggcctgctgaaaatga (SEQ ID NO: |
| | 171) |
| PsiI | TACGGCGACCACCGAGATCTACACTCTTTCCCTACACGACGCTCTTCC |
| | GATCTGATCGANNNNNNNNNNNNNNtaaggcctgctgaaaatga (SEQ ID NO: |
| | 172) |
| PsiI | TACGGCGACCACCGAGATCTACACTCTTTCCCTACACGACGCTCTTCC |
| | GATCTCGATCGANNNNNNNNNNNNNNtaaggcctgctgaaaatga (SEQ ID NO: |
| | 173) |
| Tsp45I | TACGGCGACCACCGAGATCTACACTCTTTCCCTACACGACGCTCTTCC |
| | GATCTNNNNNNNNNNNNNNattttcattatttttattataaggcctgc (SEQ ID NO: 174) |
| Tsp45I | TACGGCGACCACCGAGATCTACACTCTTTCCCTACACGACGCTCTTCC |
| | GATCTANNNNNNNNNNNNNNattttcattatttttattataaggcctgc (SEQ ID NO: |
| | 175) |
| Tsp45I | TACGGCGACCACCGAGATCTACACTCTTTCCCTACACGACGCTCTTCC |
| | GATCTGANNNNNNNNNNNNNNattttcattatttttattataaggcctgc (SEQ ID NO: |
| | 176) |
| Tsp45I | TACGGCGACCACCGAGATCTACACTCTTTCCCTACACGACGCTCTTCC |
| | GATCTCGANNNNNNNNNNNNNNattttcattatttttattataaggcctgc (SEQ ID NO: |
| | 177) |
| Tsp45I | TACGGCGACCACCGAGATCTACACTCTTTCCCTACACGACGCTCTTCC |
| | GATCTTCGANNNNNNNNNNNNNNattttcattatttttattataaggcctgc (SEQ ID NO: |
| | 178) |
| Tsp45I | TACGGCGACCACCGAGATCTACACTCTTTCCCTACACGACGCTCTTCC |
| | GATCTATCGANNNNNNNNNNNNNNattttcattatttttattataaggcctgc (SEQ ID |
| | NO: 179) |
| Tsp45I | TACGGCGACCACCGAGATCTACACTCTTTCCCTACACGACGCTCTTCC |
| | GATCTGATCGANNNNNNNNNNNNNNattttcattatttttattataaggcctgc (SEQ ID |
| | NO: 180) |
| Tsp45I | TACGGCGACCACCGAGATCTACACTCTTTCCCTACACGACGCTCTTCC |
| | GATCTCGATCGANNNNNNNNNNNNNNattttcattatttttattataaggcctgc (SEQ |
| | ID NO: 181) |

TABLE 13
Full-length exon-specific reverse primers for
KRAS non-coding strand
Sequence (5′→3′: reverse adaptor sequence, index,
second ROI-specific sequence)

CAAGCAGAAGACGGCATACGAGATGTGACTGGAGTTCAGACGTGTGCTCTTCCGA
TCTTAGCTGTATCGTCAAGGC (SEQ ID NO: 36)
CAAGCAGAAGACGGCATACGAGATGTGACTGGAGTTCAGACGTGTGCTCTTCCGA
TCTATAGCTGTATCGTCAAGGC (SEQ ID NO: 182)
CAAGCAGAAGACGGCATACGAGATGTGACTGGAGTTCAGACGTGTGCTCTTCCGA
TCTGATAGCTGTATCGTCAAGGC (SEQ ID NO: 183)
CAAGCAGAAGACGGCATACGAGATGTGACTGGAGTTCAGACGTGTGCTCTTCCGA
TCTCGATAGCTGTATCGTCAAGGC (SEQ ID NO: 184)
CAAGCAGAAGACGGCATACGAGATGTGACTGGAGTTCAGACGTGTGCTCTTCCGA
TCTTCGATAGCTGTATCGTCAAGGC (SEQ ID NO: 185)
CAAGCAGAAGACGGCATACGAGATGTGACTGGAGTTCAGACGTGTGCTCTTCCGA
TCTATCGATAGCTGTATCGTCAAGGC (SEQ ID NO: 186)
CAAGCAGAAGACGGCATACGAGATGTGACTGGAGTTCAGACGTGTGCTCTTCCGA
TCTGATCGATAGCTGTATCGTCAAGGC (SEQ ID NO: 187)
CAAGCAGAAGACGGCATACGAGATGTGACTGGAGTTCAGACGTGTGCTCTTCCGA
TCTCGATCGATAGCTGTATCGTCAAGGC (SEQ ID NO: 188)

What is claimed:

1. A method of detecting a mutation in a KRAS gene in a sample from a human subject, the method comprising:
a) digesting genomic DNA in the sample with at least one enzyme that cleaves at the 3′ end of a region of interest (ROI);
b) adding a forward adaptor and a barcode to the 3′ end of the ROI by mixing the digested genomic DNA with an adaptor-barcode primer and performing a single round of extension, wherein the adaptor-barcode primer comprises from 5′ to 3′: a forward adaptor sequence, a barcode sequence, and a first ROI-specific sequence that is complementary to the 3′ end of the digested ROI, wherein adding the forward adaptor and the barcode to the ROI produces a barcoded DNA;
c) performing linear amplification of the barcoded DNA produced in (b) using a forward adaptor primer that anneals to the forward adaptor sequence;
d) performing exponential amplification of the linearly amplified barcoded DNA produced in (c) using:
an exon-specific reverse primer comprising from 5′ to 3′: a reverse adaptor sequence, and a second ROI-specific sequence that is complementary to the 5′ end of the digested ROI,
the forward adaptor primer; and
a reverse adaptor primer that anneals to the reverse adaptor sequence; and
e) sequencing the exponentially amplified barcoded DNA produced in (d); wherein a different nucleotide in the sequenced DNA produced in (e) compared to a wild type KRAS sequence indicates that a mutation is detected.

2. The method of claim 1, wherein the ROI is on the transcribed strand of KRAS exon 1, the non-transcribed strand of KRAS exon 1, or the non-transcribed strand of KRAS exon 2.

3. The method of claim 2, wherein the ROI is on the transcribed strand of KRAS exon 1; and wherein:
the enzyme is StuI, the adaptor-barcode primer comprises the first ROI-specific sequence of SEQ ID NO: 6, and the exon-specific reverse primer comprises the second ROI-specific sequence of SEQ ID NO: 7 or SEQ ID NO: 8;
the enzyme is HinfI, the adaptor-barcode primer comprises the first ROI-specific sequence of SEQ ID NO: 25, and the exon-specific reverse primer comprises the second ROI-specific sequence of SEQ ID NO: 17;
the enzyme is MluCI, the adaptor-barcode primer comprises the first ROI-specific sequence of SEQ ID NO: 26, and the exon-specific reverse primer comprises the second ROI-specific sequence of SEQ ID NO: 17;
the enzyme is Hpy188I, the adaptor-barcode primer comprises the first ROI-specific sequence of SEQ ID NO: 27, and the exon-specific reverse primer comprises the second ROI-specific sequence of SEQ ID NO: 17;
the enzyme is AlwI, the adaptor-barcode primer comprises the first ROI-specific sequence of SEQ ID NO: 28, and the exon-specific reverse primer comprises the second ROI-specific sequence of SEQ ID NO: 17;
the enzyme is DpnII, the adaptor-barcode primer comprises the first ROI-specific sequence of SEQ ID NO: 29, and the exon-specific reverse primer comprises the second ROI-specific sequence of SEQ ID NO: 17;
the enzyme is MnlI, the adaptor-barcode primer comprises the first ROI-specific sequence of SEQ ID NO: 30, and the exon-specific reverse primer comprises the second ROI-specific sequence of SEQ ID NO: 17;
the enzyme is NsiI, the adaptor-barcode primer comprises the first ROI-specific sequence of SEQ ID NO: 31, and the exon-specific reverse primer comprises the second ROI-specific sequence of SEQ ID NO: 17;
the enzyme is HpyCH4V, the adaptor-barcode primer comprises the first ROI-specific sequence of SEQ ID NO: 32, and the exon-specific reverse primer comprises the second ROI-specific sequence of SEQ ID NO: 17; or
the enzyme is BsrI, the adaptor-barcode primer comprises the first ROI-specific sequence of SEQ ID NO: 33, and the exon-specific reverse primer comprises the second ROI-specific sequence of SEQ ID NO: 17.

4. The method of claim 2, wherein the ROI is on the non-transcribed strand of KRAS exon 1; and
wherein:
the enzyme is HinfI, the adaptor-barcode primer comprises the first ROI-specific sequence of SEQ ID NO: 9, and the exon-specific reverse primer comprises the second ROI-specific sequence of SEQ ID NO: 10;
the enzyme is PsiI, the adaptor-barcode primer comprises the first ROI-specific sequence of SEQ ID NO: 18, and the exon-specific reverse primer comprises the second ROI-specific sequence of SEQ ID NO: 8;
the enzyme is Tsp45I, the adaptor-barcode primer comprises the first ROI-specific sequence of SEQ ID NO: 19, and the exon-specific reverse primer comprises the second ROI-specific sequence of SEQ ID NO: 8;
the enzyme is selected from AflIII, PciI and FatI, the adaptor-barcode primer comprises the first ROI-specific sequence of SEQ ID NO: 20, and the exon-specific reverse primer comprises the second ROI-specific sequence of SEQ ID NO: 8;
the enzyme is selected from NspI and NlaIII, the adaptor-barcode primer comprises the first ROI-specific sequence of SEQ ID NO: 21, and the exon-specific reverse primer comprises the second ROI-specific sequence of SEQ ID NO: 8;
the enzyme is CviAII, and the adaptor-barcode primer comprises the first ROI-specific sequence of SEQ ID NO: 22, and the exon-specific reverse primer comprises the second ROI-specific sequence of SEQ ID NO: 8;
the enzyme is CviQ, the adaptor-barcode primer comprises the first ROI-specific sequence of SEQ ID NO: 23, and the exon-specific reverse primer comprises the second ROI-specific sequence of SEQ ID NO: 8; or
the enzyme is HphI, the adaptor-barcode primer comprises the first ROI-specific sequence of SEQ ID NO: 24, and the exon-specific reverse primer comprises the second ROI-specific sequence of SEQ ID NO: 8.

5. The method of claim 2, wherein the ROI is on the non-transcribed strand of KRAS exon 2; and
wherein the enzyme is XmnI, the adaptor-barcode primer comprises the first ROI-specific sequence of SEQ ID NO: 11, and the exon-specific reverse primer comprises the second ROI-specific sequence of SEQ ID NO: 12.

6. The method of claim 1, wherein the adaptor-barcode primer comprises the forward adaptor sequence of SEQ ID NO: 1; and wherein the forward adaptor primer comprises SEQ ID NO: 2.

7. The method of claim 1, wherein the exon-specific reverse primer comprises the reverse adaptor sequence of SEQ ID NO: 3; and wherein the reverse adaptor primer comprises SEQ ID NO: 4.

8. The method of claim 1, wherein the adaptor-barcode primer further comprises a first index sequence between the forward adaptor sequence and the barcode sequence; wherein the exon-specific reverse primer further comprises a second index sequence between the reverse adaptor sequence and the second ROI-specific sequence; and wherein the first index sequence and the second index sequence comprise between one and seven nucleotides.

9. The method of claim 8, wherein the first index sequence and the second index sequence are selected from A, GA, CGA, TCGA, ATCGA, GATCGA, and CGATCGA.

10. The method of claim 9, wherein the adaptor-barcode primer comprises a sequence selected from SEQ ID NOs: 37 and 41-111.

11. The method of claim 10, wherein the exon-specific reverse primer comprises a sequence selected from SEQ ID NOs: 38 and 112-118.

12. The method of claim 9, wherein the adaptor-barcode primer comprises a sequence selected from SEQ ID NOs: 34 and 119-181.

13. The method of claim 12, wherein the exon-specific reverse primer comprises a sequence selected from SEQ ID NOs: 36 and 182-188.

14. The method of claim 1, wherein performing exponential amplification in step (d) comprises performing at least 20 PCR cycles.

15. The method of claim 1, wherein the sample is a biopsy.

16. The method of claim 1, wherein the subject has or is suspected of having cancer.

17. A kit for detecting a mutation in a KRAS gene in a human subject, the kit comprising at least one set of primers comprising:
a forward adaptor primer comprising SEQ ID NO: 2;
a reverse adaptor primer comprising SEQ ID NO: 4;
an adaptor barcode primer selected from SEQ ID NOs: 34, 37, 39, 41-111, and 119-181; and
an exon-specific reverse primer selected from SEQ ID NOs: 35, 38, 40, 112-118, and 182-188;
wherein each of the at least one set of primers comprises an adaptor barcode primer and an exon-specific barcode primer that comprise a sequence that targets the same region of interest in KRAS.

18. The kit of claim 17, further comprises at least one enzyme selected from Stul, Hinfl, AlwI, BsrI, DpnII, Hpy188I, HpyCH4V, MluCI, MnlI, NsiI, Stul, AflIII, PciI, FatI, NlaIII, CviAII, CviQI, HphI, NspI, PsiI, XmnI, and Tsp45I;
wherein the at least one enzyme cleaves KRAS at the 3′ end of the region of interest that the adaptor barcode primer and the exon-specific barcode primer target.

19. The kit of claim 18, wherein:
the enzyme is StuI, the adaptor-barcode primer comprises the first ROI-specific sequence of SEQ ID NO: 6, and the exon-specific reverse primer comprises the second ROI-specific sequence of SEQ ID NO: 7 or SEQ ID NO: 8;
the enzyme is HinfI, the adaptor-barcode primer comprises the first ROI-specific sequence of SEQ ID NO: 25, and the exon-specific reverse primer comprises the second ROI-specific sequence of SEQ ID NO: 17;
the enzyme is MluCI, the adaptor-barcode primer comprises the first ROI-specific sequence of SEQ ID NO: 26, and the exon-specific reverse primer comprises the second ROI-specific sequence of SEQ ID NO: 17;
the enzyme is Hpy188I, the adaptor-barcode primer comprises the first ROI-specific sequence of SEQ ID NO: 27, and the exon-specific reverse primer comprises the second ROI-specific sequence of SEQ ID NO: 17;
the enzyme is AlwI, the adaptor-barcode primer comprises the first ROI-specific sequence of SEQ ID NO: 28, and the exon-specific reverse primer comprises the second ROI-specific sequence of SEQ ID NO: 17;
the enzyme is DpnII, the adaptor-barcode primer comprises the first ROI-specific sequence of SEQ ID NO: 29, and the exon-specific reverse primer comprises the second ROI-specific sequence of SEQ ID NO: 17;
the enzyme is MnlI, the adaptor-barcode primer comprises the first ROI-specific sequence of SEQ ID NO: 30, and the exon-specific reverse primer comprises the second ROI-specific sequence of SEQ ID NO: 17;
the enzyme is NsiI, the adaptor-barcode primer comprises the first ROI-specific sequence of SEQ ID NO: 31, and the exon-specific reverse primer comprises the second ROI-specific sequence of SEQ ID NO: 17;
the enzyme is HpyCH4V, the adaptor-barcode primer comprises the first ROI-specific sequence of SEQ ID NO: 32, and the exon-specific reverse primer comprises the second ROI-specific sequence of SEQ ID NO: 17;
the enzyme is BsrI, the adaptor-barcode primer comprises the first ROI-specific sequence of SEQ ID NO: 33, and the exon-specific reverse primer comprises the second ROI-specific sequence of SEQ ID NO: 17;
the enzyme is HinfI, the adaptor-barcode primer comprises the first ROI-specific sequence of SEQ ID NO: 9, and the exon-specific reverse primer comprises the second ROI-specific sequence of SEQ ID NO: 10;
the enzyme is PsiI, the adaptor-barcode primer comprises the first ROI-specific sequence of SEQ ID NO: 18, and the exon-specific reverse primer comprises the second ROI-specific sequence of SEQ ID NO: 8;
the enzyme is Tsp45I, the adaptor-barcode primer comprises the first ROI-specific sequence of SEQ ID NO: 19, and the exon-specific reverse primer comprises the second ROI-specific sequence of SEQ ID NO: 8;
the enzyme is selected from AflIII, PciI and FatI, the adaptor-barcode primer comprises the first ROI-specific sequence of SEQ ID NO: 20, and the exon-specific reverse primer comprises the second ROI-specific sequence of SEQ ID NO: 8;
the enzyme is selected from NspI and NlaIII, the adaptor-barcode primer comprises the first ROI-specific sequence of SEQ ID NO: 21, and the exon-specific reverse primer comprises the second ROI-specific sequence of SEQ ID NO: 8;
the enzyme is CviAII, and the adaptor-barcode primer comprises the first ROI-specific sequence of SEQ ID NO: 22, and the exon-specific reverse primer comprises the second ROI-specific sequence of SEQ ID NO: 8;
the enzyme is CviQ, the adaptor-barcode primer comprises the first ROI-specific sequence of SEQ ID NO: 23, and the exon-specific reverse primer comprises the second ROI-specific sequence of SEQ ID NO: 8;
the enzyme is HphI, the adaptor-barcode primer comprises the first ROI-specific sequence of SEQ ID NO: 24, and the exon-specific reverse primer comprises the second ROI-specific sequence of SEQ ID NO: 8; or the enzyme is XmnI, the adaptor-barcode primer comprises the first ROI-specific sequence of SEQ ID NO: 11, and the exon-specific reverse primer comprises the second ROI-specific sequence of SEQ ID NO: 12.


SEQUENCE LISTING (211 sequences)

SEQ ID NO: 1 - DNA, 53 bp, synthetic construct
  source 1..53: mol_type = other DNA
tacggcgaccaccgagatctacactctttccctacacgacgctcttccgatct

SEQ ID NO: 2 - DNA, 23 bp, synthetic construct
  source 1..23: mol_type = other DNA
aatgatacggcgaccaccgagat

SEQ ID NO: 3 - DNA, 58 bp, synthetic construct
  source 1..58: mol_type = other DNA
caagcagaagacggcatacgagatgtgactggagttcagacgtgtgctcttccgatct

SEQ ID NO: 4 - DNA, 23 bp, synthetic construct
  source 1..23: mol_type = other DNA
caagcagaagacggcatacgaga

SEQ ID NO: 5 - skipped

SEQ ID NO: 6 - DNA, 19 bp, synthetic construct
  source 1..19: mol_type = other DNA
cctgctgaaaatgactgaa

SEQ ID NO: 7 - DNA, 23 bp, synthetic construct
  source 1..23: mol_type = other DNA
ctctattgttggatcatattcgt

SEQ ID NO: 8 - DNA, 18 bp, synthetic construct
  source 1..18: mol_type = other DNA
tagctgtatcgtcaaggc

SEQ ID NO: 9 - DNA, 22 bp, synthetic construct
  source 1..22: mol_type = other DNA
ctgaattagctgtatcgtcaag

SEQ ID NO: 10 - DNA, 26 bp, synthetic construct
  source 1..26: mol_type = other DNA
atgactgaatataaacttgtggtagt

SEQ ID NO: 11 - DNA, 29 bp, synthetic construct
  source 1..29: mol_type = other DNA
tcttcaaatgatttagtattatttatggc

SEQ ID NO: 12 - DNA, 20 bp, synthetic construct
  source 1..20: mol_type = other DNA
gattcctacaggaagcaagt

SEQ ID NO: 13 - DNA, 23 bp, synthetic construct
  source 1..23: mol_type = other DNA
aggcctgctgaaaatgnaggcct

SEQ ID NO: 14 - DNA, 23 bp, synthetic construct
  source 1..23: mol_type = other DNA
tccggacgacttttacntccgga

SEQ ID NO: 15 - DNA, 17 bp, synthetic construct
  source 1..17: mol_type = other DNA
cctgctgaaaatgnagg

SEQ ID NO: 16 - DNA, 17 bp, synthetic construct
  source 1..17: mol_type = other DNA
ggacgacttttacntcc

SEQ ID NO: 17 - DNA, 26 bp, synthetic construct
  source 1..26: mol_type = other DNA
atgactgaatataaacttgtggtagt

SEQ ID NO: 18 - DNA, 19 bp, synthetic construct
  source 1..19: mol_type = other DNA
taaggcctgctgaaaatga

SEQ ID NO: 19 - DNA, 29 bp, synthetic construct
  source 1..29: mol_type = other DNA
attttcattatttttattataaggcctgc

SEQ ID NO: 20 - DNA, 39 bp, synthetic construct
  source 1..39: mol_type = other DNA
ttctaatatagtcacattttcattatttttattataagg

SEQ ID NO: 21 - DNA, 33 bp, synthetic construct
  source 1..33: mol_type = other DNA
catgttctaatatagtcacattttcattatttt

SEQ ID NO: 22 - DNA, 40 bp, synthetic construct
  source 1..40: mol_type = other DNA
gttctaatatagtcacattttcattatttttattataagg

SEQ ID NO: 23 - DNA, 21 bp, synthetic construct
  source 1..21: mol_type = other DNA
ctggtggagtatttgatagtg

SEQ ID NO: 24 - DNA, 20 bp, synthetic construct
  source 1..20: mol_type = other DNA
ttaaaaggtactggtggagt

SEQ ID NO: 25 - DNA, 22 bp, synthetic construct
  source 1..22: mol_type = other DNA
ctgaattagctgtatcgtcaag

SEQ ID NO: 26 - DNA, 18 bp, synthetic construct
  source 1..18: mol_type = other DNA
agctgtatcgtcaaggca

SEQ ID NO: 27 - DNA, 21 bp, synthetic construct
  source 1..21: mol_type = other DNA
tgaattagctgtatcgtcaag

SEQ ID NO: 28 - DNA, 20 bp, synthetic construct
  source 1..20: mol_type = other DNA
cgtccacaaaatgattctga

SEQ ID NO: 29 - DNA, 24 bp, synthetic construct
  source 1..24: mol_type = other DNA
atattcgtccacaaaatgattctg

SEQ ID NO: 30 - DNA, 20 bp, synthetic construct
  source 1..20: mol_type = other DNA
tggatcatattcgtccacaa

SEQ ID NO: 31 - DNA, 28 bp, synthetic construct
  source 1..28: mol_type = other DNA
tgcatattaaaacaagatttacctctat

SEQ ID NO: 32 - DNA, 31 bp, synthetic construct
  source 1..31: mol_type = other DNA
catattaaaacaagatttacctctattgttg

SEQ ID NO: 33 - DNA, 27 bp, synthetic construct
  source 1..27: mol_type = other DNA
accagtaatatgcatattaaaacaaga

SEQ ID NO: 34 - DNA, 86 bp, synthetic construct
  source 1..86: mol_type = other DNA
tacggcgaccaccgagatctacactctttccctacacgacgctcttccgatctnnnnnnnnnnnnnncctgctgaaaatgactgaa

SEQ ID NO: 35 - DNA, 81 bp, synthetic construct
  source 1..81: mol_type = other DNA
caagcagaagacggcatacgagatgtgactggagttcagacgtgtgctcttccgatctctctattgttggatcatattcgt

SEQ ID NO: 36 - DNA, 76 bp, synthetic construct
  source 1..76: mol_type = other DNA
caagcagaagacggcatacgagatgtgactggagttcagacgtgtgctcttccgatcttagctgtatcgtcaaggc

SEQ ID NO: 37 - DNA, 89 bp, synthetic construct
  source 1..89: mol_type = other DNA
tacggcgaccaccgagatctacactctttccctacacgacgctcttccgatctnnnnnnnnnnnnnnctgaattagctgtatcgtcaag

SEQ ID NO: 38 - DNA, 84 bp, synthetic construct
  source 1..84: mol_type = other DNA
caagcagaagacggcatacgagatgtgactggagttcagacgtgtgctcttccgatctatgactgaatataaacttgtggtagt

SEQ ID NO: 39 - DNA, 96 bp, synthetic construct
  source 1..96: mol_type = other DNA
tacggcgaccaccgagatctacactctttccctacacgacgctcttccgatctnnnnnnnnnnnnnntcttcaaatgatttagtattatttatggc

SEQ ID NO: 40 - DNA, 78 bp, synthetic construct
  source 1..78: mol_type = other DNA
caagcagaagacggcatacgagatgtgactggagttcagacgtgtgctcttccgatctgattcctacaggaagcaagt

SEQ ID NO: 41 - DNA, 90 bp, synthetic construct
  source 1..90: mol_type = other DNA
tacggcgaccaccgagatctacactctttccctacacgacgctcttccgatctannnnnnnnnnnnnnctgaattagctgtatcgtcaag

SEQ ID NO: 42 - DNA, 91 bp, synthetic construct
  source 1..91: mol_type = other DNA
tacggcgaccaccgagatctacactctttccctacacgacgctcttccgatctgannnnnnnnnnnnnnctgaattagctgtatcgtcaag

SEQ ID NO: 43 - DNA, 92 bp, synthetic construct
  source 1..92: mol_type = other DNA
tacggcgaccaccgagatctacactctttccctacacgacgctcttccgatctcgannnnnnnnnnnnnnctgaattagctgtatcgtcaag

SEQ ID NO: 44 - DNA, 93 bp, synthetic construct
  source 1..93: mol_type = other DNA
tacggcgaccaccgagatctacactctttccctacacgacgctcttccgatcttcgannnnnnnnnnnnnnctgaattagctgtatcgtcaag

SEQ ID NO: 45 - DNA, 94 bp, synthetic construct
  source 1..94: mol_type = other DNA
tacggcgaccaccgagatctacactctttccctacacgacgctcttccgatctatcgannnnnnnnnnnnnnctgaattagctgtatcgtcaag

SEQ ID NO: 46 - DNA, 95 bp, synthetic construct
  source 1..95: mol_type = other DNA
tacggcgaccaccgagatctacactctttccctacacgacgctcttccgatctgatcgannnnnnnnnnnnnnctgaattagctgtatcgtcaag

SEQ ID NO: 47 - DNA, 96 bp, synthetic construct
  source 1..96: mol_type = other DNA
tacggcgaccaccgagatctacactctttccctacacgacgctcttccgatctcgatcgannnnnnnnnnnnnnctgaattagctgtatcgtcaag

SEQ ID NO: 48 - DNA, 87 bp, synthetic construct
  source 1..87: mol_type = other DNA
tacggcgaccaccgagatctacactctttccctacacgacgctcttccgatctnnnnnnnnnnnnnncgtccacaaaatgattctga

SEQ ID NO: 49 - DNA, 88 bp, synthetic construct
  source 1..88: mol_type = other DNA
tacggcgaccaccgagatctacactctttccctacacgacgctcttccgatctannnnnnnnnnnnnncgtccacaaaatgattctga

SEQ ID NO: 50 - DNA, 89 bp, synthetic construct
  source 1..89: mol_type = other DNA
tacggcgaccaccgagatctacactctttccctacacgacgctcttccgatctgannnnnnnnnnnnnncgtccacaaaatgattctga

SEQ ID NO: 51 - DNA, 90 bp, synthetic construct
  source 1..90: mol_type = other DNA
tacggcgaccaccgagatctacactctttccctacacgacgctcttccgatctcgannnnnnnnnnnnnncgtccacaaaatgattctga

SEQ ID NO: 52 - DNA, 91 bp, synthetic construct
  source 1..91: mol_type = other DNA
tacggcgaccaccgagatctacactctttccctacacgacgctcttccgatcttcgannnnnnnnnnnnnncgtccacaaaatgattctga

SEQ ID NO: 53 - DNA, 92 bp, synthetic construct
  source 1..92: mol_type = other DNA
tacggcgaccaccgagatctacactctttccctacacgacgctcttccgatctatcgannnnnnnnnnnnnncgtccacaaaatgattctga

SEQ ID NO: 54 - DNA, 93 bp, synthetic construct
  source 1..93: mol_type = other DNA
tacggcgaccaccgagatctacactctttccctacacgacgctcttccgatctgatcgannnnnnnnnnnnnncgtccacaaaatgattctga

SEQ ID NO: 55 - DNA, 94 bp, synthetic construct
  source 1..94: mol_type = other DNA
tacggcgaccaccgagatctacactctttccctacacgacgctcttccgatctcgatcgannnnnnnnnnnnnncgtccacaaaatgattctga

SEQ ID NO: 56 - DNA, 94 bp, synthetic construct
  source 1..94: mol_type = other DNA
tacggcgaccaccgagatctacactctttccctacacgacgctcttccgatctnnnnnnnnnnnnnnaccagtaatatgcatattaaaacaaga

SEQ ID NO: 57 - DNA, 95 bp, synthetic construct
  source 1..95: mol_type = other DNA
tacggcgaccaccgagatctacactctttccctacacgacgctcttccgatctannnnnnnnnnnnnnaccagtaatatgcatattaaaacaaga

SEQ ID NO: 58 - DNA, 96 bp, synthetic construct
  source 1..96: mol_type = other DNA
tacggcgaccaccgagatctacactctttccctacacgacgctcttccgatctgannnnnnnnnnnnnnaccagtaatatgcatattaaaacaaga

SEQ ID NO: 59 - DNA, 97 bp, synthetic construct
  source 1..97: mol_type = other DNA
tacggcgaccaccgagatctacactctttccctacacgacgctcttccgatctcgannnnnnnnnnnnnnaccagtaatatgcatattaaaacaaga

SEQ ID NO: 60 - DNA, 98 bp, synthetic construct
  source 1..98: mol_type = other DNA
tacggcgaccaccgagatctacactctttccctacacgacgctcttccgatcttcgannnnnnnnnnnnnnaccagtaatatgcatattaaaacaaga

SEQ ID NO: 61 - DNA, 99 bp, synthetic construct
  source 1..99: mol_type = other DNA
tacggcgaccaccgagatctacactctttccctacacgacgctcttccgatctatcgannnnnnnnnnnnnnaccagtaatatgcatattaaaacaaga

SEQ ID NO: 62 - DNA, 100 bp, synthetic construct
  source 1..100: mol_type = other DNA
tacggcgaccaccgagatctacactctttccctacacgacgctcttccgatctgatcgannnnnnnnnnnnnnaccagtaatatgcatattaaaacaaga

SEQ ID NO: 63 - DNA, 101 bp, synthetic construct
  source 1..101: mol_type = other DNA
tacggcgaccaccgagatctacactctttccctacacgacgctcttccgatctcgatcgannnnnnnnnnnnnnaccagtaatatgcatattaaaacaaga

SEQ ID NO: 64 - DNA, 91 bp, synthetic construct
  source 1..91: mol_type = other DNA
tacggcgaccaccgagatctacactctttccctacacgacgctcttccgatctnnnnnnnnnnnnnnatattcgtccacaaaatgattctg

SEQ ID NO: 65 - DNA, 92 bp, synthetic construct
  source 1..92: mol_type = other DNA
tacggcgaccaccgagatctacactctttccctacacgacgctcttccgatctannnnnnnnnnnnnnatattcgtccacaaaatgattctg

SEQ ID NO: 66 - DNA, 93 bp, synthetic construct
  source 1..93: mol_type = other DNA
tacggcgaccaccgagatctacactctttccctacacgacgctcttccgatctgannnnnnnnnnnnnnatattcgtccacaaaatgattctg

SEQ ID NO: 67 - DNA, 94 bp, synthetic construct
  source 1..94: mol_type = other DNA
tacggcgaccaccgagatctacactctttccctacacgacgctcttccgatctcgannnnnnnnnnnnnnatattcgtccacaaaatgattctg

SEQ ID NO: 68 - DNA, 95 bp, synthetic construct
  source 1..95: mol_type = other DNA
tacggcgaccaccgagatctacactctttccctacacgacgctcttccgatcttcgannnnnnnnnnnnnnatattcgtccacaaaatgattctg

SEQ ID NO: 69 - DNA, 96 bp, synthetic construct
  source 1..96: mol_type = other DNA
tacggcgaccaccgagatctacactctttccctacacgacgctcttccgatctatcgannnnnnnnnnnnnnatattcgtccacaaaatgattctg

SEQ ID NO: 70 - DNA, 97 bp, synthetic construct
  source 1..97: mol_type = other DNA
tacggcgaccaccgagatctacactctttccctacacgacgctcttccgatctgatcgannnnnnnnnnnnnnatattcgtccacaaaatgattctg

SEQ ID NO: 71 - DNA, 98 bp, synthetic construct
  source 1..98: mol_type = other DNA
tacggcgaccaccgagatctacactctttccctacacgacgctcttccgatctcgatcgannnnnnnnnnnnnnatattcgtccacaaaatgattctg

SEQ ID NO: 72 - DNA, 88 bp, synthetic construct
  source 1..88: mol_type = other DNA
tacggcgaccaccgagatctacactctttccctacacgacgctcttccgatctnnnnnnnnnnnnnntgaattagctgtatcgtcaag

SEQ ID NO: 73 - DNA, 89 bp, synthetic construct
  source 1..89: mol_type = other DNA
tacggcgaccaccgagatctacactctttccctacacgacgctcttccgatctannnnnnnnnnnnnntgaattagctgtatcgtcaag

SEQ ID NO: 74 - DNA, 90 bp, synthetic construct
  source 1..90: mol_type = other DNA
tacggcgaccaccgagatctacactctttccctacacgacgctcttccgatctgannnnnnnnnnnnnntgaattagctgtatcgtcaag

SEQ ID NO: 75 - DNA, 91 bp, synthetic construct
  source 1..91: mol_type = other DNA
tacggcgaccaccgagatctacactctttccctacacgacgctcttccgatctcgannnnnnnnnnnnnntgaattagctgtatcgtcaag

SEQ ID NO: 76 - DNA, 92 bp, synthetic construct
  source 1..92: mol_type = other DNA
tacggcgaccaccgagatctacactctttccctacacgacgctcttccgatcttcgannnnnnnnnnnnnntgaattagctgtatcgtcaag

SEQ ID NO: 77 - DNA, 93 bp, synthetic construct
  source 1..93: mol_type = other DNA
tacggcgaccaccgagatctacactctttccctacacgacgctcttccgatctatcgannnnnnnnnnnnnntgaattagctgtatcgtcaag

SEQ ID NO: 78 - DNA, 94 bp, synthetic construct
  source 1..94: mol_type = other DNA
tacggcgaccaccgagatctacactctttccctacacgacgctcttccgatctgatcgannnnnnnnnnnnnntgaattagctgtatcgtcaag

SEQ ID NO: 79 - DNA, 95 bp, synthetic construct
  source 1..95: mol_type = other DNA
tacggcgaccaccgagatctacactctttccctacacgacgctcttccgatctcgatcgannnnnnnnnnnnnntgaattagctgtatcgtcaag

SEQ ID NO: 80 - DNA, 98 bp, synthetic construct
  source 1..98: mol_type = other DNA
tacggcgaccaccgagatctacactctttccctacacgacgctcttccgatctnnnnnnnnnnnnnncatattaaaacaagatttacctctattgttg

SEQ ID NO: 81 - DNA, 99 bp, synthetic construct
  source 1..99: mol_type = other DNA
tacggcgaccaccgagatctacactctttccctacacgacgctcttccgatctannnnnnnnnnnnnncatattaaaacaagatttacctctattgttg

SEQ ID NO: 82 - DNA, 100 bp, synthetic construct
  source 1..100: mol_type = other DNA
tacggcgaccaccgagatctacactctttccctacacgacgctcttccgatctgannnnnnnnnnnnnncatattaaaacaagatttacctctattgttg

SEQ ID NO: 83 - DNA, 101 bp, synthetic construct
  source 1..101: mol_type = other DNA
tacggcgaccaccgagatctacactctttccctacacgacgctcttccgatctcgannnnnnnnnnnnnncatattaaaacaagatttacctctattgttg

SEQ ID NO: 84 - DNA, 102 bp, synthetic construct
  source 1..102: mol_type = other DNA
tacggcgaccaccgagatctacactctttccctacacgacgctcttccgatcttcgannnnnnnnnnnnnncatattaaaacaagatttacctctattgttg

SEQ ID NO: 85 - DNA, 103 bp, synthetic construct
  source 1..103: mol_type = other DNA
tacggcgaccaccgagatctacactctttccctacacgacgctcttccgatctatcgannnnnnnnnnnnnncatattaaaacaagatttacctctattgttg

SEQ ID NO: 86 - DNA, 104 bp, synthetic construct
  source 1..104: mol_type = other DNA
tacggcgaccaccgagatctacactctttccctacacgacgctcttccgatctgatcgannnnnnnnnnnnnncatattaaaacaagatttacctctattgttg

SEQ ID NO: 87 - DNA, 105 bp, synthetic construct
  source 1..105: mol_type = other DNA
tacggcgaccaccgagatctacactctttccctacacgacgctcttccgatctcgatcgannnnnnnnnnnnnncatattaaaacaagatttacctctattgttg

SEQ ID NO: 88 - DNA, 85 bp, synthetic construct
  source 1..85: mol_type = other DNA
tacggcgaccaccgagatctacactctttccctacacgacgctcttccgatctnnnnnnnnnnnnnnagctgtatcgtcaaggca

SEQ ID NO: 89 - DNA, 86 bp, synthetic construct
  source 1..86: mol_type = other DNA
tacggcgaccaccgagatctacactctttccctacacgacgctcttccgatctannnnnnnnnnnnnnagctgtatcgtcaaggca

SEQ ID NO: 90 - DNA, 87 bp, synthetic construct
  source 1..87: mol_type = other DNA
tacggcgaccaccgagatctacactctttccctacacgacgctcttccgatctgannnnnnnnnnnnnnagctgtatcgtcaaggca

SEQ ID NO: 91 - DNA, 88 bp, synthetic construct
  source 1..88: mol_type = other DNA
tacggcgaccaccgagatctacactctttccctacacgacgctcttccgatctcgannnnnnnnnnnnnnagctgtatcgtcaaggca

SEQ ID NO: 92 - DNA, 89 bp, synthetic construct
  source 1..89: mol_type = other DNA
tacggcgaccaccgagatctacactctttccctacacgacgctcttccgatcttcgannnnnnnnnnnnnnagctgtatcgtcaaggca

SEQ ID NO: 93 - DNA, 90 bp, synthetic construct
  source 1..90: mol_type = other DNA
tacggcgaccaccgagatctacactctttccctacacgacgctcttccgatctatcgannnnnnnnnnnnnnagctgtatcgtcaaggca

SEQ ID NO: 94 - DNA, 91 bp, synthetic construct
  source 1..91: mol_type = other DNA
tacggcgaccaccgagatctacactctttccctacacgacgctcttccgatctgatcgannnnnnnnnnnnnnagctgtatcgtcaaggca

SEQ ID NO: 95 - DNA, 92 bp, synthetic construct
  source 1..92: mol_type = other DNA
tacggcgaccaccgagatctacactctttccctacacgacgctcttccgatctcgatcgannnnnnnnnnnnnnagctgtatcgtcaaggca

SEQ ID NO: 96 - DNA, 87 bp, synthetic construct
  source 1..87: mol_type = other DNA
tacggcgaccaccgagatctacactctttccctacacgacgctcttccgatctnnnnnnnnnnnnnntggatcatattcgtccacaa

SEQ ID NO: 97 - DNA, 88 bp, synthetic construct
  source 1..88: mol_type = other DNA
tacggcgaccaccgagatctacactctttccctacacgacgctcttccgatctannnnnnnnnnnnnntggatcatattcgtccacaa

SEQ ID NO: 98 - DNA, 89 bp, synthetic construct
  source 1..89: mol_type = other DNA
tacggcgaccaccgagatctacactctttccctacacgacgctcttccgatctgannnnnnnnnnnnnntggatcatattcgtccacaa

SEQ ID NO: 99 - DNA, 90 bp, synthetic construct
  source 1..90: mol_type = other DNA
tacggcgaccaccgagatctacactctttccctacacgacgctcttccgatctcgannnnnnnnnnnnnntggatcatattcgtccacaa

SEQ ID NO: 100 - DNA, 91 bp, synthetic construct
  source 1..91: mol_type = other DNA
tacggcgaccaccgagatctacactctttccctacacgacgctcttccgatcttcgannnnnnnnnnnnnntggatcatattcgtccacaa

SEQ ID NO: 101 - DNA, 92 bp, synthetic construct
  source 1..92: mol_type = other DNA
tacggcgaccaccgagatctacactctttccctacacgacgctcttccgatctatcgannnnnnnnnnnnnntggatcatattcgtccacaa

SEQ ID NO: 102 - DNA, 93 bp, synthetic construct
  source 1..93: mol_type = other DNA
tacggcgaccaccgagatctacactctttccctacacgacgctcttccgatctgatcgannnnnnnnnnnnnntggatcatattcgtccacaa

SEQ ID NO: 103 - DNA, 94 bp, synthetic construct
  source 1..94: mol_type = other DNA
tacggcgaccaccgagatctacactctttccctacacgacgctcttccgatctcgatcgannnnnnnnnnnnnntggatcatattcgtccacaa

SEQ ID NO: 104 - DNA, 95 bp, synthetic construct
  source 1..95: mol_type = other DNA
tacggcgaccaccgagatctacactctttccctacacgacgctcttccgatctnnnnnnnnnnnnnntgcatattaaaacaagatttacctctat

SEQ ID NO: 105 - DNA, 96 bp, synthetic construct
  source 1..96: mol_type = other DNA
tacggcgaccaccgagatctacactctttccctacacgacgctcttccgatctannnnnnnnnnnnnntgcatattaaaacaagatttacctctat

SEQ ID NO: 106 - DNA, 97 bp, synthetic construct
  source 1..97: mol_type = other DNA
tacggcgaccaccgagatctacactctttccctacacgacgctcttccgatctgannnnnnnnnnnnnntgcatattaaaacaagatttacctctat

SEQ ID NO: 107 - DNA, 98 bp, synthetic construct
  source 1..98: mol_type = other DNA
tacggcgaccaccgagatctacactctttccctacacgacgctcttccgatctcgannnnnnnnnnnnnntgcatattaaaacaagatttacctctat

SEQ ID NO: 108 - DNA, 99 bp, synthetic construct
  source 1..99: mol_type = other DNA
tacggcgaccaccgagatctacactctttccctacacgacgctcttccgatcttcgannnnnnnnnnnnnntgcatattaaaacaagatttacctctat

SEQ ID NO: 109 - DNA, 100 bp, synthetic construct
  source 1..100: mol_type = other DNA
tacggcgaccaccgagatctacactctttccctacacgacgctcttccgatctatcgannnnnnnnnnnnnntgcatattaaaacaagatttacctctat

SEQ ID NO: 110 - DNA, 101 bp, synthetic construct
  source 1..101: mol_type = other DNA
tacggcgaccaccgagatctacactctttccctacacgacgctcttccgatctgatcgannnnnnnnnnnnnntgcatattaaaacaagatttacctctat

SEQ ID NO: 111 - DNA, 102 bp, synthetic construct
  source 1..102: mol_type = other DNA
tacggcgaccaccgagatctacactctttccctacacgacgctcttccgatctcgatcgannnnnnnnnnnnnntgcatattaaaacaagatttacctctat

SEQ ID NO: 112 - DNA, 85 bp, synthetic construct
  source 1..85: mol_type = other DNA
caagcagaagacggcatacgagatgtgactggagttcagacgtgtgctcttccgatctaatgactgaatataaacttgtggtagt

SEQ ID NO: 113 - DNA, 86 bp, synthetic construct
  source 1..86: mol_type = other DNA
caagcagaagacggcatacgagatgtgactggagttcagacgtgtgctcttccgatctgaatgactgaatataaacttgtggtagt

SEQ ID NO: 114 - DNA, 87 bp, synthetic construct
  source 1..87: mol_type = other DNA
caagcagaagacggcatacgagatgtgactggagttcagacgtgtgctcttccgatctcgaatgactgaatataaacttgtggtagt

SEQ ID NO: 115 - DNA, 88 bp, synthetic construct
  source 1..88: mol_type = other DNA
caagcagaagacggcatacgagatgtgactggagttcagacgtgtgctcttccgatcttcgaatgactgaatataaacttgtggtagt

SEQ ID NO: 116 - DNA, 89 bp, synthetic construct
  source 1..89: mol_type = other DNA
caagcagaagacggcatacgagatgtgactggagttcagacgtgtgctcttccgatctatcgaatgactgaatataaacttgtggtagt

SEQ ID NO: 117 - DNA, 90 bp, synthetic construct
  source 1..90: mol_type = other DNA
caagcagaagacggcatacgagatgtgactggagttcagacgtgtgctcttccgatctgatcgaatgactgaatataaacttgtggtagt

SEQ ID NO: 118 - DNA, 91 bp, synthetic construct
  source 1..91: mol_type = other DNA
caagcagaagacggcatacgagatgtgactggagttcagacgtgtgctcttccgatctcgatcgaatgactgaatataaacttgtggtagt

SEQ ID NO: 119 - DNA, 87 bp, synthetic construct
  source 1..87: mol_type = other DNA
tacggcgaccaccgagatctacactctttccctacacgacgctcttccgatctannnnnnnnnnnnnncctgctgaaaatgactgaa

SEQ ID NO: 120 - DNA, 88 bp, synthetic construct
  source 1..88: mol_type = other DNA
tacggcgaccaccgagatctacactctttccctacacgacgctcttccgatctgannnnnnnnnnnnnncctgctgaaaatgactgaa

SEQ ID NO: 121 - DNA, 89 bp, synthetic construct
  source 1..89: mol_type = other DNA
tacggcgaccaccgagatctacactctttccctacacgacgctcttccgatctcgannnnnnnnnnnnnncctgctgaaaatgactgaa

SEQ ID NO: 122 - DNA, 90 bp, synthetic construct
  source 1..90: mol_type = other DNA
tacggcgaccaccgagatctacactctttccctacacgacgctcttccgatcttcgannnnnnnnnnnnnncctgctgaaaatgactgaa

SEQ ID NO: 123 - DNA, 91 bp, synthetic construct
  source 1..91: mol_type = other DNA
tacggcgaccaccgagatctacactctttccctacacgacgctcttccgatctatcgannnnnnnnnnnnnncctgctgaaaatgactgaa

SEQ ID NO: 124 - DNA, 92 bp, synthetic construct
  source 1..92: mol_type = other DNA
tacggcgaccaccgagatctacactctttccctacacgacgctcttccgatctgatcgannnnnnnnnnnnnncctgctgaaaatgactgaa

SEQ ID NO: 125 - DNA, 93 bp, synthetic construct
  source 1..93: mol_type = other DNA
tacggcgaccaccgagatctacactctttccctacacgacgctcttccgatctcgatcgannnnnnnnnnnnnncctgctgaaaatgactgaa

SEQ ID NO: 126 - DNA, 106 bp, synthetic construct
  source 1..106: mol_type = other DNA
tacggcgaccaccgagatctacactctttccctacacgacgctcttccgatctnnnnnnnnnnnnnnttctaatatagtcacattttcattatttttattataagg

SEQ ID NO: 127 - DNA, 107 bp, synthetic construct
  source 1..107: mol_type = other DNA
tacggcgaccaccgagatctacactctttccctacacgacgctcttccgatctannnnnnnnnnnnnnttctaatatagtcacattttcattatttttattataagg

SEQ ID NO: 128 - DNA, 108 bp, synthetic construct
  source 1..108: mol_type = other DNA
tacggcgaccaccgagatctacactctttccctacacgacgctcttccgatctgannnnnnnnnnnnnnttctaatatagtcacattttcattatttttattataagg

SEQ ID NO: 129 - DNA, 109 bp, synthetic construct
  source 1..109: mol_type = other DNA
tacggcgaccaccgagatctacactctttccctacacgacgctcttccgatctcgannnnnnnnnnnnnnttctaatatagtcacattttcattatttttattataagg

SEQ ID NO: 130 - DNA, 110 bp, synthetic construct
  source 1..110: mol_type = other DNA
tacggcgaccaccgagatctacactctttccctacacgacgctcttccgatcttcgannnnnnnnnnnnnnttctaatatagtcacattttcattatttttattataagg

SEQ ID NO: 131 - DNA, 111 bp, synthetic construct
  source 1..111: mol_type = other DNA
tacggcgaccaccgagatctacactctttccctacacgacgctcttccgatctatcgannnnnnnnnnnnnnttctaatatagtcacattttcattatttttattataagg

SEQ ID NO: 132 - DNA, 112 bp, synthetic construct
  source 1..112: mol_type = other DNA
tacggcgaccaccgagatctacactctttccctacacgacgctcttccgatctgatcgannnnnnnnnnnnnnttctaatatagtcacattttcattatttttattataagg

SEQ ID NO: 133 - DNA, 113 bp, synthetic construct
  source 1..113: mol_type = other DNA
tacggcgaccaccgagatctacactctttccctacacgacgctcttccgatctcgatcgannnnnnnnnnnnnnttctaatatagtcacattttcattatttttattataagg

SEQ ID NO: 134 - DNA, 107 bp, synthetic construct
  source 1..107: mol_type = other DNA
tacggcgaccaccgagatctacactctttccctacacgacgctcttccgatctnnnnnnnnnnnnnngttctaatatagtcacattttcattatttttattataagg

SEQ ID NO: 135 - DNA, 108 bp, synthetic construct
  source 1..108: mol_type = other DNA
tacggcgaccaccgagatctacactctttccctacacgacgctcttccgatctannnnnnnnnnnnnngttctaatatagtcacattttcattatttttattataagg

SEQ ID NO: 136 - DNA, 109 bp, synthetic construct
  source 1..109: mol_type = other DNA
tacggcgaccaccgagatctacactctttccctacacgacgctcttccgatctgannnnnnnnnnnnnngttctaatatagtcacattttcattatttttattataagg

SEQ ID NO: 137 - DNA, 110 bp, synthetic construct
  source 1..110: mol_type = other DNA
tacggcgaccaccgagatctacactctttccctacacgacgctcttccgatctcgannnnnnnnnnnnnngttctaatatagtcacattttcattatttttattataagg

SEQ ID NO: 138 - DNA, 111 bp, synthetic construct
  source 1..111: mol_type = other DNA
tacggcgaccaccgagatctacactctttccctacacgacgctcttccgatcttcgannnnnnnnnnnnnngttctaatatagtcacattttcattatttttattataagg

SEQ ID NO: 139 - DNA, 112 bp, synthetic construct
  source 1..112: mol_type = other DNA
tacggcgaccaccgagatctacactctttccctacacgacgctcttccgatctatcgannnnnnnnnnnnnngttctaatatagtcacattttcattatttttattataagg

SEQ ID NO: 140 - DNA, 113 bp, synthetic construct
  source 1..113: mol_type = other DNA
tacggcgaccaccgagatctacactctttccctacacgacgctcttccgatctgatcgannnnnnnnnnnnnngttctaatatagtcacattttcattatttttattataagg

SEQ ID NO: 141 - DNA, 114 bp, synthetic construct
  source 1..114: mol_type = other DNA
tacggcgaccaccgagatctacactctttccctacacgacgctcttccgatctcgatcgannnnnnnnnnnnnngttctaatatagtcacattttcattatttttattataagg

SEQ ID NO: 142 - DNA, 88 bp, synthetic construct
  source 1..88: mol_type = other DNA
tacggcgaccaccgagatctacactctttccctacacgacgctcttccgatctnnnnnnnnnnnnnnctggtggagtatttgatagtg

SEQ ID NO: 143 - DNA, 89 bp, synthetic construct
  source 1..89: mol_type = other DNA
tacggcgaccaccgagatctacactctttccctacacgacgctcttccgatctannnnnnnnnnnnnnctggtggagtatttgatagtg

SEQ ID NO: 144 - DNA, 90 bp, synthetic construct
  source 1..90: mol_type = other DNA
tacggcgaccaccgagatctacactctttccctacacgacgctcttccgatctgannnnnnnnnnnnnnctggtggagtatttgatagtg

SEQ ID NO: 145 - DNA, 91 bp, synthetic construct
  source 1..91: mol_type = other DNA
tacggcgaccaccgagatctacactctttccctacacgacgctcttccgatctcgannnnnnnnnnnnnnctggtggagtatttgatagtg

SEQ ID NO: 146 - DNA, 92 bp, synthetic construct
  source 1..92: mol_type = other DNA
tacggcgaccaccgagatctacactctttccctacacgacgctcttccgatcttcgannnnnnnnnnnnnnctggtggagtatttgatagtg

SEQ ID NO: 147 - DNA, 93 bp, synthetic construct
  source 1..93: mol_type = other DNA
tacggcgaccaccgagatctacactctttccctacacgacgctcttccgatctatcgannnnnnnnnnnnnnctggtggagtatttgatagtg

SEQ ID NO: 148 - DNA, 94 bp, synthetic construct
  source 1..94: mol_type = other DNA
tacggcgaccaccgagatctacactctttccctacacgacgctcttccgatctgatcgannnnnnnnnnnnnnctggtggagtatttgatagtg

SEQ ID NO: 149 - DNA, 95 bp, synthetic construct
  source 1..95: mol_type = other DNA
tacggcgaccaccgagatctacactctttccctacacgacgctcttccgatctcgatcgannnnnnnnnnnnnnctggtggagtatttgatagtg

SEQ ID NO: 150 - DNA, 87 bp, synthetic construct
  source 1..87: mol_type = other DNA
tacggcgaccaccgagatctacactctttccctacacgacgctcttccgatctnnnnnnnnnnnnnnttaaaaggtactggtggagt

SEQ ID NO: 151 - DNA, 88 bp, synthetic construct
  source 1..88: mol_type = other DNA
tacggcgaccaccgagatctacactctttccctacacgacgctcttccgatctannnnnnnnnnnnnnttaaaaggtactggtggagt

SEQ ID NO: 152 - DNA, 89 bp, synthetic construct
  source 1..89: mol_type = other DNA
tacggcgaccaccgagatctacactctttccctacacgacgctcttccgatctgannnnnnnnnnnnnnttaaaaggtactggtggagt

SEQ ID NO: 153 - DNA, 90 bp, synthetic construct
  source 1..90: mol_type = other DNA
tacggcgaccaccgagatctacactctttccctacacgacgctcttccgatctcgannnnnnnnnnnnnnttaaaaggtactggtggagt

SEQ ID NO: 154 - DNA, 91 bp, synthetic construct
  source 1..91: mol_type = other DNA
tacggcgaccaccgagatctacactctttccctacacgacgctcttccgatcttcgannnnnnnnnnnnnnttaaaaggtactggtggagt

SEQ ID NO: 155 - DNA, 92 bp, synthetic construct
  source 1..92: mol_type = other DNA
tacggcgaccaccgagatctacactctttccctacacgacgctcttccgatctatcgannnnnnnnnnnnnnttaaaaggtactggtggagt

SEQ ID NO: 156 - DNA, 93 bp, synthetic construct
  source 1..93: mol_type = other DNA
tacggcgaccaccgagatctacactctttccctacacgacgctcttccgatctgatcgannnnnnnnnnnnnnttaaaaggtactggtggagt

SEQ ID NO: 157 - DNA, 94 bp, synthetic construct
  source 1..94: mol_type = other DNA
tacggcgaccaccgagatctacactctttccctacacgacgctcttccgatctcgatcgannnnnnnnnnnnnnttaaaaggtactggtggagt

SEQ ID NO: 158 - DNA, 100 bp, synthetic construct
  source 1..100: mol_type = other DNA
tacggcgaccaccgagatctacactctttccctacacgacgctcttccgatctnnnnnnnnnnnnnncatgttctaatatagtcacattttcattatttt

SEQ ID NO: 159 - DNA, 101 bp, synthetic construct
  source 1..101: mol_type = other DNA
tacggcgaccaccgagatctacactctttccctacacgacgctcttccgatctannnnnnnnnnnnnncatgttctaatatagtcacattttcattatttt

SEQ ID NO: 160 - DNA, 102 bp, synthetic construct
  source 1..102: mol_type = other DNA
tacggcgaccaccgagatctacactctttccctacacgacgctcttccgatctgannnnnnnnnnnnnncatgttctaatatagtcacattttcattatttt

SEQ ID NO: 161 - DNA, 103 bp, synthetic construct
  source 1..103: mol_type = other DNA
tacggcgaccaccgagatctacactctttccctacacgacgctcttccgatctcgannnnnnnnnnnnnncatgttctaatatagtcacattttcattatttt

SEQ ID NO: 162 - DNA, 104 bp, synthetic construct
  source 1..104: mol_type = other DNA
tacggcgaccaccgagatctacactctttccctacacgacgctcttccgatcttcgannnnnnnnnnnnnncatgttctaatatagtcacattttcattatttt

SEQ ID NO: 163 - DNA, 105 bp, synthetic construct
  source 1..105: mol_type = other DNA
tacggcgaccaccgagatctacactctttccctacacgacgctcttccgatctatcgannnnnnnnnnnnnncatgttctaatatagtcacattttcattatttt

SEQ ID NO: 164 - DNA, 106 bp, synthetic construct
  source 1..106: mol_type = other DNA
tacggcgaccaccgagatctacactctttccctacacgacgctcttccgatctgatcgannnnnnnnnnnnnncatgttctaatatagtcacattttcattatttt

SEQ ID NO: 165 - DNA, 107 bp, synthetic construct
  source 1..107: mol_type = other DNA
tacggcgaccaccgagatctacactctttccctacacgacgctcttccgatctcgatcgannnnnnnnnnnnnncatgttctaatatagtcacattttcattatttt

SEQ ID NO: 166 - DNA, 86 bp, synthetic construct
  source 1..86: mol_type = other DNA
tacggcgaccaccgagatctacactctttccctacacgacgctcttccgatctnnnnnnnnnnnnnntaaggcctgctgaaaatga

SEQ ID NO: 167 - DNA, 87 bp, synthetic construct
  source 1..87: mol_type = other DNA
tacggcgaccaccgagatctacactctttccctacacgacgctcttccgatctannnnnnnnnnnnnntaaggcctgctgaaaatga

SEQ ID NO: 168 - DNA, 88 bp, synthetic construct
  source 1..88: mol_type = other DNA
tacggcgaccaccgagatctacactctttccctacacgacgctcttccgatctgannnnnnnnnnnnnntaaggcctgctgaaaatga

SEQ ID NO: 169 - DNA, 89 bp, synthetic construct
  source 1..89: mol_type = other DNA
tacggcgaccaccgagatctacactctttccctacacgacgctcttccgatctcgannnnnnnnnnnnnntaaggcctgctgaaaatga

SEQ ID NO: 170 - DNA, 90 bp, synthetic construct
  source 1..90: mol_type = other DNA
tacggcgaccaccgagatctacactctttccctacacgacgctcttccgatcttcgannnnnnnnnnnnnntaaggcctgctgaaaatga

SEQ ID NO: 171 - DNA, 91 bp, synthetic construct
  source 1..91: mol_type = other DNA
tacggcgaccaccgagatctacactctttccctacacgacgctcttccgatctatcgannnnnnnnnnnnnntaaggcctgctgaaaatga

SEQ ID NO: 172 - DNA, 92 bp, synthetic construct
  source 1..92: mol_type = other DNA
tacggcgaccaccgagatctacactctttccctacacgacgctcttccgatctgatcgannnnnnnnnnnnnntaaggcctgctgaaaatga

SEQ ID NO: 173 - DNA, 93 bp, synthetic construct
  source 1..93: mol_type = other DNA
tacggcgaccaccgagatctacactctttccctacacgacgctcttccgatctcgatcgannnnnnnnnnnnnntaaggcctgctgaaaatga

SEQ ID NO: 174 - DNA, 96 bp, synthetic construct
  source 1..96: mol_type = other DNA
tacggcgaccaccgagatctacactctttccctacacgacgctcttccgatctnnnnnnnnnnnnnnattttcattatttttattataaggcctgc

SEQ ID NO: 175 - DNA, 97 bp, synthetic construct
  source 1..97: mol_type = other DNA
tacggcgaccaccgagatctacactctttccctacacgacgctcttccgatctannnnnnnnnnnnnnattttcattatttttattataaggcctgc

SEQ ID NO: 176 - DNA, 98 bp, synthetic construct
  source 1..98: mol_type = other DNA
tacggcgaccaccgagatctacactctttccctacacgacgctcttccgatctgannnnnnnnnnnnnnattttcattatttttattataaggcctgc

SEQ ID NO: 177 - DNA, 99 bp, synthetic construct
  source 1..99: mol_type = other DNA
tacggcgaccaccgagatctacactctttccctacacgacgctcttccgatctcgannnnnnnnnnnnnnattttcattatttttattataaggcctgc

SEQ ID NO: 178 - DNA, 100 bp, synthetic construct
  source 1..100: mol_type = other DNA
tacggcgaccaccgagatctacactctttccctacacgacgctcttccgatcttcgannnnnnnnnnnnnnattttcattatttttattataaggcctgc

SEQ ID NO: 179 - DNA, 101 bp, synthetic construct
  source 1..101: mol_type = other DNA
tacggcgaccaccgagatctacactctttccctacacgacgctcttccgatctatcgannnnnnnnnnnnnnattttcattatttttattataaggcctgc

SEQ ID NO: 180 - DNA, 102 bp, synthetic construct
  source 1..102: mol_type = other DNA
tacggcgaccaccgagatctacactctttccctacacgacgctcttccgatctgatcgannnnnnnnnnnnnnattttcattatttttattataaggcctgc

SEQ ID NO: 181 - DNA, 103 bp, synthetic construct
  source 1..103: mol_type = other DNA
tacggcgaccaccgagatctacactctttccctacacgacgctcttccgatctcgatcgannnnnnnnnnnnnnattttcattatttttattataaggcctgc

SEQ ID NO: 182 - DNA, 77 bp, synthetic construct
  source 1..77: mol_type = other DNA
caagcagaagacggcatacgagatgtgactggagttcagacgtgtgctcttccgatctatagctgtatcgtcaaggc

SEQ ID NO: 183 - DNA, 78 bp, synthetic construct
  source 1..78: mol_type = other DNA
caagcagaagacggcatacgagatgtgactggagttcagacgtgtgctcttccgatctgatagctgtatcgtcaaggc

SEQ ID NO: 184 - DNA, 79 bp, synthetic construct
  source 1..79: mol_type = other DNA
caagcagaagacggcatacgagatgtgactggagttcagacgtgtgctcttccgatctcgatagctgtatcgtcaaggc

SEQ ID NO: 185 - DNA, 80 bp, synthetic construct
  source 1..80: mol_type = other DNA
caagcagaagacggcatacgagatgtgactggagttcagacgtgtgctcttccgatcttcgatagctgtatcgtcaaggc

SEQ ID NO: 186 - DNA, 81 bp, synthetic construct
  source 1..81: mol_type = other DNA
caagcagaagacggcatacgagatgtgactggagttcagacgtgtgctcttccgatctatcgatagctgtatcgtcaaggc

SEQ ID NO: 187 - DNA, 82 bp, synthetic construct
  source 1..82: mol_type = other DNA
caagcagaagacggcatacgagatgtgactggagttcagacgtgtgctcttccgatctgatcgatagctgtatcgtcaaggc

SEQ ID NO: 188 - DNA, 83 bp, synthetic construct
  source 1..83: mol_type = other DNA
caagcagaagacggcatacgagatgtgactggagttcagacgtgtgctcttccgatctcgatcgatagctgtatcgtcaaggc

SEQ ID NO: 189 - DNA, 40 bp, synthetic construct
  source 1..40: mol_type = other DNA
aattgcggccgcccagggggtatagcgtactatgcagaat

SEQ ID NO: 190 - DNA, 22 bp, synthetic construct
  source 1..22: mol_type = other DNA
cattttcagcaggccttacaat

SEQ ID NO: 191 - DNA, 39 bp, synthetic construct
  source 1..39: mol_type = other DNA
attgtaaggcctgctgaaagaagagtataaacttgtggt

SEQ ID NO: 192 - DNA, 32 bp, synthetic construct
  source 1..32: mol_type = other DNA
cagggtcgactcacataactgtacaccttgtc

SEQ ID NO: 193 - DNA, 28 bp, synthetic construct
  source 1..28: mol_type = other DNA
aattgtcgacttagacgtcaggtggcac

SEQ ID NO: 194 - DNA, 30 bp, synthetic construct
  source 1..30: mol_type = other DNA
ttaagcggccgcgtttgcgtattgggcgct

SEQ ID NO: 195 - DNA, 19 bp, synthetic construct
  source 1..19: mol_type = other DNA
cctgctgaaaatgactgag

SEQ ID NO: 196 - DNA, 22 bp, synthetic construct
  source 1..22: mol_type = other DNA
atctttttcaaagcggctggct

SEQ ID NO: 197 - DNA, 20 bp, synthetic construct
  source 1..20: mol_type = other DNA
tcaggactcctacaggaaac

SEQ ID NO: 198 - DNA, 20 bp, synthetic construct
  source 1..20: mol_type = other DNA
taggtggctcacctgtactg

SEQ ID NO: 199 - DNA, 60 bp, synthetic construct
  source 1..60: mol_type = other DNA
aagcagaagacggcatacgagatcggtctcggcattcctgctgaaccgctcttccgatct

SEQ ID NO: 200 - DNA, 23 bp, synthetic construct
  source 1..23: mol_type = other DNA
ctctatcgtagggtcatactcat

SEQ ID NO: 201 - DNA, 27 bp, synthetic construct
  source 1..27: mol_type = other DNA
tattatttttattgtaaggcctgctga

SEQ ID NO: 202 - DNA, 20 bp, synthetic construct
  source 1..20: mol_type = other DNA
gactcctacaggaaacaagt

SEQ ID NO: 203 - DNA, 24 bp, synthetic construct
  source 1..24: mol_type = other DNA
ctttcttattcaacttaaacccac

SEQ ID NO: 204 - DNA, 21 bp, synthetic construct
  source 1..21: mol_type = other DNA
ctaagccgtgttgttttgcag

SEQ ID NO: 205 - DNA, 21 bp, synthetic construct
  source 1..21: mol_type = other DNA
atggagaaacctgtctcttgg

SEQ ID NO: 206 - DNA, 21 bp, synthetic construct
  source 1..21: mol_type = other DNA
ctcatgtactggtccctcatt

SEQ ID NO: 207 - DNA, 19 bp, synthetic construct
  source 1..19: mol_type = other DNA
ccagcgtcgtgattagcga

SEQ ID NO: 208 - DNA, 20 bp, synthetic construct
  source 1..20: mol_type = other DNA
ccagcaggtcagcaaagaac

SEQ ID NO: 209 - DNA, 20 bp, synthetic construct
  source 1..20: mol_type = other DNA
gcaagagcgccttgacgata

SEQ ID NO: 210 - DNA, 23 bp, synthetic construct
  source 1..23: mol_type = other DNA
catgtactggtccctcattgcac

SEQ ID NO: 211 - DNA, 66 bp, synthetic construct
  source 1..66: mol_type = other DNA
caagcagaagacggcatacgagatnnnnnnnngtgactggagttcagacgtgtgctcttccgatct